(19) United States
(12) Patent Application Publication
(10) Pub. No.: US 2023/0109758 A1
(43) Pub. Date: Apr. 13, 2023

(54) GENETICALLY MODIFIED CLOSTRIDIUM BACTERIA, PREPARATION AND USES OF SAME

(71) Applicant: IFP ENERGIES NOUVELLES, RUEIL-MALMAISON (FR)

(72) Inventors: RÈMI HOCQ, PARIS (FR); GWLADYS CHARTIER, CHATOU (FR); FRANÇOIS WASELS, METZ (FR); NICOLAS LOPES FERREIRA, CROISILLES (FR)

(21) Appl. No.: 17/414,337

(22) PCT Filed: Dec. 20, 2019
(86) PCT No.: PCT/FR2019/053227

§ 371 Date: Jan. 14, 2022

(30) Foreign Application Priority Data
Dec. 20, 2018 (FR) 1873492

(51) Int. Cl. C12N 15/74; C12P 7/04; C12N 15/113; C12N 9/22

(52) U.S. Cl. CPC C12N 15/74; C12P 7/04; C12N 15/1137; C12Y 203/01028; C12N 9/22; C12N 2310/20; C12N 2800/80

(57) ABSTRACT

The present invention relates to the genetic modification of bacteria of the genus Clostridium, typically solventogenic bacteria of the genus Clostridium, in particular bacteria possessing in the wild type a gene encoding an amphenicol-O-acetyltransferase. It thus relates to methods, tools and kits allowing such a genetic modification, in particular the removal or modification of a sequence encoding or controlling the transcription of an amphenicol-O-acetyltransferase, to the genetically modified bacteria obtained and to uses thereof, in particular for producing a solvent, preferably on an industrial scale.

### CROSS-REFERENCE TO RELATED APPLICATION

This application is the U.S. national stage application of International Patent Application No. PCT/FR2019/053227, filed Dec. 20, 2019.

### STATEMENT REGARDING SEQUENCE LISTING

The Sequence Listing for this application is labeled “Seq-List.txt” which was created on Jun. 4, 2021 and is 358 KB. The entire content of the sequence listing is incorporated herein by reference in its entirety.

The present invention relates to the genetic modification of bacteria of the genus Clostridium, typically solventogenic bacteria of the genus Clostridium, in particular bacteria which in the wild type possess a gene encoding an amphenicol-O-acetyltransferase. It thus relates to methods, tools and kits allowing such a genetic modification, in particular the removal or modification of a sequence encoding, or controlling the transcription of a sequence encoding, an amphenicol-O-acetyltransferase, the genetically modified bacteria obtained and uses thereof, in particular to produce a solvent, preferably on an industrial scale.

### TECHNOLOGICAL BACKGROUND

The genus Clostridium contains Gram-positive, strict anaerobic, spore-forming bacteria belonging to the phylum Firmicutes. Clostridia are an important group for the scientific community for several reasons. The first is that a number of serious diseases (e.g., tetanus, botulism) are due to infections of pathogenic members of this family (Gonzales et al., 2014) The second is the possibility of using so-called acidogenic or solventogenic strains in biotechnology (John & Wood, 1986, and Moon et al., 2016). These non-pathogenic Clostridia naturally have the ability to transform a wide variety of sugars to produce chemical species of interest, and more particularly acetone, butanol, and ethanol (John & Wood, 1986) during a fermentative process known as ABE. Similarly, IBE fermentation, in which acetone is reduced in varying proportions to isopropanol, is possible in certain particular species (Chen et al., 1986, George et al., 1983) thanks to the presence in the genome of these strains of genes encoding secondary alcohol dehydrogenases (s-ADH; Ismael et al., 1993, Hiu et al., 1987).

The solventogenic Clostridia species show significant phenotypic similarities, which made them difficult to classify before the emergence of modern sequencing techniques (Rogers et al., 2006). With the possibility of sequencing the complete genomes of these bacteria, it is now possible to classify this bacterial genus into 4 major species: C. acetobutylicum, C. saccharoperbutylacetonicum, C. saccharobutylicum and C. beijerinckii. A recent publication has classified these solventogenic Clostridia into 4 main clades after comparative analysis of the complete genomes of 30 strains (FIG. 1).

In particular, these groups separate the species C. acetobutylicum and C. beijerinckii with C. acetobutylicum ATCC 824 (also designated DSM 792 or LMG 5710) and C. beijerinckii NCIMB 8052 as model strains for the study of ABE-type fermentation.

Clostridium strains naturally capable of performing IBE fermentation are few and mostly belong to the species Clostridium beijerinckii (see Zhang et al., 2018, Table 1). These strains are typically selected from C. butylicum LMD 27.6, C. aurantibutylicum NCIB 10659, C. beijerinckii LMD 27.6, C. beijerinckii VPI2968, C. beijerinckii NRRL B-593, C. beijerinckii ATCC 6014, C. beijerinckii McClung 3081, C. isopropylicum IAM 19239, C. beijerinckii DSM 6423, C. sp. A1424, C. beijerinckii optinoii, and C. beijerinckii BGS1.

However, to date there is no strain of Clostridium bacteria naturally capable of producing isopropanol, in particular naturally capable of performing IBE fermentation, which has been genetically modified, in particular a strain genetically modified so as to make it sensitive to an antibiotic belonging to the class of amphenicols such as chloramphenicol or thiamphenicol, preferably to enable optimized production of isopropanol.

### SUMMARY OF THE INVENTION

The inventors describe, in the context of the present invention and for the first time, a genetically modified C. beijerinckii bacterium, as well as the tools allowing genetic modification of bacteria of the genus Clostridium, typically solventogenic bacteria of the genus Clostridium naturally (i.e., in the wild type) capable of producing isopropanol, in particular naturally capable of performing IBE fermentation, in particular bacteria comprising in the wild type a gene conferring on the bacterium resistance to one or more antibiotics, in particular a gene encoding an amphenicol-O-acetyltransferase, for example a chloramphenicol-O-acetyltransferase or a thiamphenicol-O-acetyltransferase.

A preferred genetically modified bacterium in accordance with the invention is a bacterium not expressing an enzyme conferring resistance to one or more antibiotics, in particular a bacterium not expressing an amphenicol-O-acetyltransferase, for example a bacterium lacking or unable to express the catB gene.

A preferred genetically modified bacterium in accordance with the invention is the bacterium identified in the present description as C. beijerinckii DSM 6423 ΔcatB as registered under deposit number LMG P-31151 (also identified as Clostridium beijerinckii IFP962 delta catB) with the Belgian Co-ordinated Collections of Micro-organisms (“BCCM”, K. L. Ledeganckstraat 35, B-9000 Gent—Belgium) on 6 Dec. 2018. The description also relates to any derived bacteria, clone, mutant or genetically modified version thereof.

A particular subject matter described by the inventors is a nucleic acid recognizing (binding at least in part), and preferably targeting, i.e., recognizing and allowing the cleavage, in the genome of a bacterium of interest, of at least one strand of i) a sequence encoding, ii) a sequence controlling the transcription of a sequence encoding, or iii) a sequence flanking the sequence encoding, an enzyme allowing said bacterium of interest to grow in a culture medium containing an antibiotic, typically an antibiotic belonging to the class of amphenicols, preferably selected from chloramphenicol, thiamphenicol, azidamfenicol and florfenicol, typically an amphenicol-O-acetyltransferase such as a chloramphenicol-O-acetyltransferase or a thiamphenicol-O-acetyltransferase.

The inventors also describe the use of such a nucleic acid to transform and/or genetically modify a bacterium of the genus Clostridium, preferably a bacterium of the genus Clostridium naturally capable of producing isopropanol, in particular a bacterium of the genus Clostridium capable of performing IBE fermentation.

In particular, the inventors describe the use of a nucleic acid recognizing the catB gene of sequence SEQ ID NO: 18 or a sequence at least 70% identical thereto within the genome of C. beijerinckii DSM 6423 to transform and/or genetically modify a C. beijerinckii DSM 6423 bacterium.

The bacterium capable of producing isopropanol in the wild type may be, for example, a bacterium selected from a C. beijerinckii bacterium, a C. diolis bacterium, a C. puniceum bacterium, a C. butyricum bacterium, a C. saccharoperbutylacetonicum bacterium, a C. botulinum bacterium, a C. drakei bacterium, a C. scatologenes bacterium, a C. perfringens bacterium, and a C. tunisiense bacterium, preferably a bacterium selected from a C. beijerinckii bacterium, a C. diolis bacterium, a C. puniceum bacterium, and a C. saccharoperbutylacetonicum bacterium. A particularly preferred bacterium naturally capable of producing isopropanol is a C. beijerinckii bacterium.

According to a particular aspect, the nucleic acid recognizing, and preferably targeting, i) a sequence encoding an amphenicol-O-acetyltransferase, ii) a sequence controlling the transcription of such a sequence, or iii) a sequence flanking such a sequence, is used to transform a subclade of C. beijerinckii selected from DSM 6423, LMG 7814, LMG 7815, NRRL B-593, NCCB 27006 and a subclade having at least 97% identity to strain DSM 6423.

The inventors further describe a process for transforming, and preferably genetically modifying, a bacterium of the genus Clostridium. This process comprises a step of transforming said bacterium by introducing into this bacterium a nucleic acid recognizing, and preferably targeting, i) a sequence encoding, ii) a sequence controlling the transcription of a sequence encoding, or iii) a sequence flanking a sequence encoding, an enzyme of interest, preferably an amphenicol-O-acetyltransferase. This process is typically performed using a genetic modification tool, for example using a genetic modification tool selected from a CRISPR tool, a group II intron-based tool and an allelic exchange tool. Bacteria transformed and genetically modified using such a process, an example of which is the C. beijerinckii DSM 6423 ΔcatB bacterium, are also described.

Another aspect described by the inventors relates to the use of a genetically modified bacterium in accordance with the invention, preferably the C. beijerinckii DSM 6423 ΔcatB bacterium as registered under deposit number LMG P-31151, or a genetically modified version thereof, to produce a solvent, preferably isopropanol, or a mixture of solvents, preferably on an industrial scale.

Finally, the description relates to kits, in particular to a kit comprising a nucleic acid described in the present text and a genetic modification tool, in particular a genetic modification tool selected from the elements of a genetic tool selected from a CRISPR tool, a group II intron-based tool and an allelic exchange tool; a nucleic acid as guide RNA (gRNA); a nucleic acid as repair template; at least one primer pair; and an inducer allowing the expression of a protein encoded by said tool.

### DETAILED DESCRIPTION OF THE INVENTION

Although used industrially for more than a century, knowledge of bacteria belonging to the genus Clostridium is limited by the difficulties encountered in genetically modifying them.

Different genetic tools have been designed in recent years to optimize strains of this genus, the latest generation being based on the use of clustered regularly interspaced short palindromic repeats (CRISPR)/CRISPR-associated protein (Cas) technology. This method is based on the use of an enzyme called a nuclease (typically a Cas-type nuclease in the case of the CRISPR/Cas genetic tool, such as the Cas9 protein from Streptococcus pyogenes), which, guided by an RNA molecule, will make a double-stranded cut in a DNA molecule (target sequence of interest). The sequence of the guide RNA (gRNA) will determine the cutting site of the nuclease, conferring very high specificity on it (FIG. 17).

Since a double-stranded cut in an essential DNA molecule is lethal to an organism, its survival will depend on its ability to repair it (see for example Cui & Bikard, 2016). In bacteria of the genus Clostridium, repair of a double-stranded break depends on a homologous recombination mechanism requiring an intact copy of the cleaved sequence. By providing the bacterium with a DNA fragment allowing this repair while modifying the original sequence, it is possible to force the microorganism to integrate the desired changes into its genome. The modification performed must no longer allow the targeting of genomic DNA by the Cas9-gRNA ribonucleoprotein complex, via the modification of the target sequence or the PAM site (FIG. 18).

Different approaches have been described to try to make this genetic tool functional in bacteria of the genus Clostridium. These microorganisms are indeed known to be difficult to modify genetically because of their low transformation and homologous recombination frequencies. Several approaches are based on the use of Cas9, expressed constitutively in C. beijerinckii and C. ljungdahlii (Wang et al., 2015; Huang et al., 2016) or under the control of an inducible promoter in C. beijerinckii, C. saccharoperbutylacetonicum and C. authoethanogenum (Wang et al., 2016; Nagaraju et al., 2016; Wang et al., 2017). Other authors have described the use of a modified version of the nuclease, Cas9n, which makes single-stranded, instead of double-stranded, cuts within the genome (Xu et al., 2015; Li et al., 2016). This choice is due to observations that Cas9 is too toxic for use in bacteria of the genus Clostridium under the experimental conditions tested. Most of the tools described above rely on the use of a single plasmid. Finally, it is also possible to use endogenous CRISPR/Cas systems when they have been identified within the genome of the microorganism, as for example in C. pasteurianum (Pyne et al., 2016).

Unless they use (as in the last case described above) the endogenous machinery of the strain to be modified, tools based on CRISPR technology have the major disadvantage of significantly limiting the size of the nucleic acid of interest (and thus the number of coding sequences or genes) that can be inserted into the bacterial genome (about 1.8 kb at best according to Xu et al., 2015).

The inventors have developed and described a more powerful genetic tool for modifying bacteria, suitable for bacteria, typically bacteria belonging to the phylum Firmicutes, in particular bacteria of the genus Clostridium, based on the use of two distinct nucleic acids, typically two plasmids (see WO2017064439, Wasels et al., 2017 and FIG. 3) that notably solves this problem. In a particular embodiment, the first nucleic acid of this tool allows the expression of cas9 and a second nucleic acid, specific to the modification to be performed, contains one or more gRNA expression cassettes as well as a repair template allowing the replacement of a portion of the bacterial DNA targeted by Cas9 by a sequence of interest. The toxicity of the system is limited by placing cas9 and/or the gRNA expression cassette(s) under the control of inducible promoters. The inventors have recently improved this tool by making it possible to very significantly increase the transformation efficiency and thus the obtention, in useful number and quantity (in particular in a context of selection of robust strains for industrial scale production), of genetically modified bacteria of interest (see FR 18/54835). In this improved tool at least one nucleic acid comprises a sequence encoding an anti-CRISPR protein (“acr”), placed under the control of an inducible promoter. This anti-CRISPR protein represses the activity of the DNA endonuclease/guide RNA complex. The expression of the protein is regulated to allow its expression only during the transformation stage of the bacterium.

In the context of the present description, bacterium belonging to the phylum Firmicutes is understood to mean bacteria belonging to the class Clostridia, Mollicutes, Bacilli or Togobacteria, preferably to the class Clostridia or Bacilli.

Particular bacteria belonging to the phylum Firmicutes include for example bacteria of the genus Clostridium, bacteria of the genus Bacillus or bacteria of the genus Lactobacillus.

“Bacterium of the genus Bacillus” means in particular B. amyloliquefaciens, B. thurigiensis, B. coagulans, B. cereus, B. anthracis or B. subtilis.

“Bacterium of the genus Clostridium” means in particular Clostridium species of industrial interest, typically solventogenic or acetogenic bacteria of the genus Clostridium. The expression “bacterium of the genus Clostridium” encompasses wild-type bacteria as well as strains derived therefrom, genetically modified with the aim of improving their performance (for example overexpressing the ctfA, ctfB and adc genes) without having been exposed to the CRISPR system.

“Clostridium species of industrial interest” means species capable of producing, by fermentation, solvents and acids such as butyric acid or acetic acid, from sugars or monosaccharides, typically from sugars comprising 5 carbon atoms such as xylose arabinose or fructose, from sugars comprising 6 carbon atoms such as glucose or mannose, from polysaccharides such as cellulose or hemicelluloses and/or from any other source of carbon that can be assimilated and used by bacteria of the genus Clostridium (CO, CO₂, and methanol for example). Examples of solventogenic bacteria of interest are bacteria of the genus Clostridium producing acetone, butanol, ethanol and/or isopropanol, such as the strains identified in the literature as “ABE strain” [strains performing fermentations allowing the production of acetone, butanol and ethanol] and “IBE strain” [strains performing fermentations allowing the production of isopropanol (by reduction of acetone), butanol and ethanol]. Solventogenic bacteria of the genus Clostridium can be selected for example from C. acetobutylicum, C. cellulolyticum, C. phytofermentans, C. beijerinckii, C. saccharobutylicum, C. saccharoperbutylacetonicum, C. sporogenes, C. butyricum, C. aurantibutyricum and C. tyrobutyricum, most preferably from C. acetobutylicum, C. beijerinckii, C. butyricum, C. tyrobutyricum and C. cellulolyticum, and even more preferably from C. acetobutylicum and C. beijerinckii.

Bacteria of the genus Clostridium naturally producing isopropanol, typically possessing in their genome an adh gene encoding a primary/secondary alcohol dehydrogenase that reduces acetone to isopropanol, are distinguished both genetically and functionally from bacteria capable of ABE fermentation in their natural state.

The inventors have advantageously succeeded, in the context of the present invention, in genetically modifying a naturally isopropanol-producing bacterium of the genus Clostridium, the bacterium C. beijerinckii DSM 6423, as well as the reference strain C. acetobutylicum DSM 792.

The inventors thus describe, for the first time, a solventogenic bacterium of the genus Clostridium naturally (i.e., in the wild type) capable of producing isopropanol, in particular naturally capable of performing IBE fermentation, which has been genetically modified, as well as the tools, in particular the genetic tools, which enabled it to be obtained. These tools have the advantage of considerably facilitating the transformation and genetic modification of bacteria capable, in the wild type, of producing isopropanol, in particular of performing IBE fermentation, in particular those carrying a gene encoding an enzyme responsible for resistance to an antibiotic.

Part of the work described in the experimental part was performed within a strain capable of IBE fermentation, i.e., C. beijerinckii strain DSM 6423, the genome and transcriptomic analysis of which were recently described by the inventors (Máté de Gerando et al., 2018).

In particular, during the genome assembly of this strain, the inventors discovered, in addition to the chromosome, the presence of mobile genetic elements (accession number PRJEB11626—see Worldwide Website: ebi.ac.uk/ena/data/view/PRJEB11626): two natural plasmids (pNF1 and pNF2) and a linear bacteriophage (Φ6423).

In a particular embodiment of the invention, the inventors have succeeded in deleting from C. beijerinckii strain DSM 6423 its natural plasmid pNF2.

In another particular embodiment, they succeeded in deleting the upp gene originally present on the chromosome of C. beijerinckii strain DSM 6423. These experiments thus demonstrate the possible use of the tools and more generally of the technology described in the present text by the inventors to genetically modify a bacterium capable, in the wild type, of producing isopropanol, in particular to perform IBE fermentation.

In a particularly advantageous embodiment, the inventors have in particular succeeded in making sensitive to an amphenicol a bacterium that is a natural carrier (carrier in the wild type) of a gene encoding an enzyme responsible for resistance to these antibiotics.

Examples of amphenicols of interest in the context of the invention are chloramphenicol, thiamphenicol, azidamfenicol and florfenicol (Schwarz S. et al., 2004), in particular chloramphenicol and thiamphenicol.

A first aspect of the invention thus relates to a genetic tool that can be used to genetically transform and/or modify a bacterium of interest, typically a bacterium as described in the present text belonging to the phylum Firmicutes, for example a bacterium of the genus Clostridium, of the genus Bacillus or of the genus Lactobacillus, preferably a solventogenic bacterium of the genus Clostridium naturally (i.e., in the wild type) capable of producing isopropanol, in particular naturally capable of performing IBE fermentation, preferably a bacterium naturally resistant to one or more antibiotics, such as a C. beijerinckii bacterium. A preferred bacterium has in the wild type both a bacterial chromosome and at least one DNA molecule distinct from the chromosomal DNA.

According to a particular aspect, this genetic tool consists of a nucleic acid (also identified in the present text as “nucleic acid of interest”) recognizing (binding at least in part), and preferably targeting, i.e., recognizing and allowing the cleavage, in the genome of a bacterium of interest, of at least one strand of i) a sequence encoding an enzyme allowing the bacterium of interest to grow in a culture medium containing an antibiotic to which it confers resistance ii) a sequence controlling the transcription of a sequence encoding an enzyme allowing the bacterium of interest to grow in a culture medium containing an antibiotic to which it confers resistance, or iii) a sequence flanking a sequence encoding an enzyme allowing the bacterium of interest to grow in a culture medium containing an antibiotic to which it confers resistance. This nucleic acid of interest is typically used in the context of the present invention to delete the recognized sequence from the genome of the bacterium or to modify its expression, for example to modulate/regulate its expression, in particular to inhibit it, preferably to modify it so as to render said bacterium incapable of expressing a protein, in particular a functional protein, from said sequence. The recognized sequence is also identified in the present text as “target sequence” or “targeted sequence”.

In a particular embodiment, the nucleic acid of interest comprises at least one region complementary to the target sequence that is 100% identical or at least 80% identical, preferably 85%, 90%, 95%, 96%, 97%, 98% or 99% identical to the targeted region/portion/sequence of DNA within the bacterial genome and is capable of hybridizing to all or part of the sequence complementary to said region/portion/sequence, typically to a sequence comprising at least 1 nucleotide, preferably at least 1, 2, 3, 4, 5, 10, 14, 15, 20, 25, 30, 35 or 40 nucleotides, typically between 1, 10 or 20 and 1000 nucleotides, for example between 1, 10 or 20 and 900, 800, 700, 600, 500, 400, 300 or 200 nucleotides, between 1, 10 or 20 and 100 nucleotides, between 1, 10 or 20 and 50 nucleotides, or between 1, 10 or 20 and 40 nucleotides, for example between 10 and 40 nucleotides, between 10 and 30 nucleotides, between 10 and 20 nucleotides, between 20 and 30 nucleotides, between 15 and 40 nucleotides, between 15 and 30 nucleotides or between 15 and 20 nucleotides, preferably to a sequence comprising 14, 15, 16, 17, 18, 19, 20, 21, 22, 23, 24, 25, 26, 27, 28, 29 or 30 nucleotides. The region complementary to the target sequence present within the nucleic acid of interest may correspond to the “SDS” region of a guide RNA (gRNA) used in a CRISPR tool as described in the present text.

In another particular embodiment, the nucleic acid of interest comprises at least two regions each complementary to a target sequence, 100% identical or at least 80% identical, preferably at least 85%, 90%, 95%, 96%, 97%, 98% or 99% identical to said targeted region/portion/sequence of DNA within the bacterial genome. These regions are capable of hybridizing to all or part of the sequence complementary to said region/portion/sequence, typically to a sequence as described above comprising at least 1 nucleotide, preferably at least 100 nucleotides, typically between 100 and 1000 nucleotides. The regions complementary to the target sequence present within the nucleic acid of interest can recognize, preferably target, the 5′ and 3′ flanking regions of the targeted sequence in a genetic modification tool as described in the present text, for example the CLOSTRON genetic tool, the TARGETRON genetic tool, or an ACE type allelic exchange tool.

Typically, the target sequence is a sequence encoding an amphenicol-O-acetyltransferase, for example a chloramphenicol-O-acetyltransferase or a thiamphenicol-O-acetyltransferase, controlling the transcription of such a sequence or flanking such a sequence in the genome of a bacterium of interest of the genus Clostridium capable of growing in a culture medium containing one or more antibiotics belonging to the class of amphenicols, for example chloramphenicol and/or thiamphenicol.

In a particular embodiment, the recognized sequence is the sequence SEQ ID NO: 18 corresponding to the catB gene (CIBE_3859) encoding a chloramphenicol acetyltransferase from C. beijerinckii DSM 6423 or an amino acid sequence at least 70%, 75%, 80%, 85%, 90% or 95% identical to said chloramphenicol-O-acetyltransferase, or a sequence comprising all or at least 70%, 75%, 80%, 85%, 90%, 95%, 96%, 97%, 98% or 99% of the sequence SEQ ID NO: 18. In other words, the recognized sequence may be a sequence comprising at least 1 nucleotide, preferably at least 1, 2, 3, 4, 5, 10, 15, 20, 25, 30, 35 or 40 nucleotides, typically between 1 and 40 nucleotides, preferably a sequence comprising 14, 15, 16, 17, 18, 19, 20, 21, 22, 23, 24, 25, 26, 27, 28, 29 or 30 nucleotides of the sequence SEQ ID NO: 18.

Examples of amino acid sequences at least 70% identical to chloramphenicol-O-acetyltransferase encoded by the sequence SEQ ID NO: 18 correspond to the sequences identified in the NCBI database under the following entries: WP_077843937.1, SEQ ID NO: 44 (WP_063843219.1), SEQ ID NO: 45 (WP_078116092.1), SEQ ID NO: 46 (WP_077840383.1), SEQ ID NO: 47 (WP_077307770.1), SEQ ID NO: 48 (WP_103699368.1), SEQ ID NO: 49 (WP_087701812.1), SEQ ID NO: 50 (WP_017210112.1), SEQ ID NO: 51 (WP_077831818.1), SEQ ID NO: 52 (WP_012059398.1), SEQ ID NO: 53 (WP_077363893.1), SEQ ID NO: 54 (WP_015393553.1), SEQ ID NO: 55 (WP_023973814.1), SEQ ID NO: 56 (WP_026887895.1), SEQ ID NO: 57 (AWK51568.1), SEQ ID NO: 58 (WP_003359882.1), SEQ ID NO: 59 (WP_091687918.1), SEQ ID NO: 60 (WP_055668544.1), SEQ ID NO: 61 (KGK90159.1), SEQ ID NO: 62 (WP_032079033.1), SEQ ID NO: 63 (WP_029163167.1), SEQ ID NO: 64 (WP_017414356.1), SEQ ID NO: 65 (WP_073285202.1), SEQ ID NO: 66 (WP_063843220.1), and SEQ ID NO: 67 (WP_021281995.1).

Examples of amino acid sequences at least 75% identical to chloramphenicol-O-acetyltransferase encoded by the sequence SEQ ID NO: 18 correspond to sequences WP_077843937.1, WP_063843219.1, WP_078116092.1, WP_077840383.1, WP_077307770.1, WP_103699368.1, WP_087701812.1, WP_017210112.1, WP_077831818.1, WP_012059398.1, WP_077363893.1, WP_015393553.1, WP_023973814.1, WP_026887895.1 AWK51568.1, WP_003359882.1, WP_091687918.1, WP_055668544.1 and KGK90159.1.

Examples of amino acid sequences at least 90% identical to chloramphenicol acetyltransferase encoded by the sequence SEQ ID NO: 18, are sequences WP_077843937.1, WP_063843219.1, WP_078116092.1, WP_077840383.1, WP_077307770.1, WP_103699368.1, WP_087701812.1, WP_017210112.1, WP_077831818.1, WP_012059398.1, WP_077363893.1, WP_015393553.1, WP_023973814.1, WP_026887895.1 and AWK51568.1.

Examples of amino acid sequences at least 95% identical to chloramphenicol acetyltransferase encoded by the sequence SEQ ID NO: 18 correspond to sequences WP_077843937.1, WP_063843219.1, WP_078116092.1, WP_077840383.1, WP_077307770.1, WP_103699368.1, WP_087701812.1, WP_017210112.1, WP_077831818.1, WP_012059398.1, WP_077363893.1, WP_015393553.1, WP_023973814.1, and WP_026887895.1.

Preferred amino acid sequences that are at least 99% identical to the chloramphenicol-O-acetyltransferase encoded by the sequence SEQ ID NO: 18 are sequences WP_077843937.1, SEQ ID NO: 44 (WP_063843219.1) and SEQ ID NO: 45 (WP_078116092.1).

A particular sequence identical to SEQ ID NO: 18 is the sequence identified in the NCBI database under entry WP_077843937.1.

In a particular embodiment, the target sequence is the sequence SEQ ID NO: 68 corresponding to the catQ gene encoding a chloramphenicol-O-acetyltransferase from C. perfringens the amino acid sequence of which corresponds to SEQ ID NO: 66 (WP_063843220.1), or a sequence at least 70%, 75%, 80%, 85%, 90% or 95% identical to said chloramphenicol-O-acetyltransferase, or a sequence comprising all or at least 70%, 75%, 80%, 85%, 90%, 95%, 96%, 97%, 98% or 99% of the sequence SEQ ID NO: 68.

In other words, the recognized sequence can be a sequence comprising at least 1 nucleotide, preferably at least 1, 2, 3, 4, 5, 10, 15, 20, 25, 30, 35 or 40 nucleotides, typically between 1 and 40 nucleotides, preferably a sequence comprising 14, 15, 16, 17, 18, 19, 20, 21, 22, 23, 24, 25, 26, 27, 28, 29 or 30 nucleotides of the sequence SEQ ID NO: 68.

In still another particular embodiment, the recognized sequence is selected from a nucleic acid sequence catB (SEQ ID NO: 18), catQ (SEQ ID NO: 68), catD (SEQ ID NO: 69, Schwarz S. et al., 2004) or catP (SEQ ID NO: 70, Schwarz S. et al., 2004) known to the person skilled in the art, naturally present within a bacterium of the genus Clostridium or artificially introduced into such a bacterium.

As indicated above, according to another embodiment, the target sequence can also be a sequence controlling the transcription of a coding sequence as described above (encoding an enzyme allowing the bacterium of interest to grow in a culture medium containing an antibiotic to which it confers resistance), typically a promoter sequence, for example the promoter sequence (SEQ ID NO: 73) of the catB gene or that (SEQ ID NO: 74) of the catQ gene.

The nucleic acid of interest, used as a genetic tool, then recognizes, and is therefore typically able to bind to, a sequence controlling the transcription of a coding sequence as described above.

According to another embodiment, the target sequence may be a sequence flanking a coding sequence as described above (encoding an enzyme allowing the bacterium of interest to grow in a culture medium containing an antibiotic to which it confers resistance), for example a sequence flanking the catB gene of sequence SEQ ID NO: 18 or a sequence at least 70% identical thereto. Such a flanking sequence typically comprises 1, 10 or 20 and 1000 nucleotides, for example between 1, 10 or 20 and 900, 800, 700, 600, 500, 400, 300 or 200 nucleotides, between 1, 10 or 20 and 100 nucleotides, between 1, 10 or 20 and 50 nucleotides or between 1, 10 or 20 and 40 nucleotides, for example between 10 and 40 nucleotides, between 10 and 30 nucleotides, between 10 and 20 nucleotides, between 20 and 30 nucleotides, between 15 and 40 nucleotides, between 15 and 30 nucleotides or between 15 and 20 nucleotides.

According to a particular aspect, the target sequence corresponds to the pair of sequences flanking such coding sequence, each flanking sequence typically comprising at least 20 nucleotides, typically between 100 and 1000 nucleotides, preferably between 200 and 800 nucleotides.

In the sense of the invention, “nucleic acid” means any natural, synthetic, semi-synthetic or recombinant DNA or RNA molecule, optionally chemically modified (i.e., comprising non-natural bases, modified nucleotides comprising, for example, a modified linkage, modified bases and/or modified sugars), or optimized in such a way that the codons of the transcripts synthesized from the coding sequences are the codons that are most frequently found in a bacterium of the genus Clostridium with a view to its use therein. In the case of the genus Clostridium, the optimized codons are typically codons rich in adenine (“A”) and thymine (“T”) bases.

In the peptide sequences described in this document, the amino acids are represented by their one-letter code according to the following nomenclature: C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan and Y: tyrosine.

In the context of the present invention, the nucleic acid of interest, used as a genetic tool to transform and/or genetically modify a bacterium of interest, is a DNA fragment i) recognizing a sequence encoding, ii) controlling the transcription of a sequence encoding, or iii) flanking a sequence encoding, an enzyme of interest, preferably an amphenicol-O-acetyltransferase, for example a chloramphenicol-O-acetyltransferase or a thiamphenicol-O-acetyltransferase, in the genome of a bacterium of the genus Clostridium, in particular a solventogenic bacterium of the genus Clostridium, which is naturally capable of producing isopropanol, in particular is naturally capable of performing IBE fermentation.

The bacterium capable of naturally producing isopropanol may be, for example, a bacterium selected from a C. beijerinckii, a C. diolis bacterium, a C. puniceum bacterium, a C. butyricum bacterium, a C. saccharoperbutylacetonicum bacterium, a C. botulinum bacterium, a C. drakei bacterium, a C. scatologenes bacterium, a C. perfringens bacterium, and a C. tunisiense bacterium, preferably a bacterium selected from a C. beijerinckii bacterium, a C. diolis bacterium, a C. puniceum bacterium and a C. saccharoperbutylacetonicum bacterium. A particularly preferred bacterium capable of producing isopropanol in the wild type is a C. beijerinckii bacterium.

According to a particular aspect, the bacterium of the genus Clostridium is a C. beijerinckii bacterium whose subclade is selected from DSM 6423, LMG 7814, LMG 7815, NCCB 27006 and a subclade having at least 90%, 95%, 96%, 97%, 98% or 99% identity with strain DSM 6423.

As previously indicated, the nucleic acid of interest in accordance with the invention is capable of deleting the sequence (“target sequence”) recognized in the genome of the bacterium or of modifying its expression, for example of modulating it, in particular of inhibiting it, preferably of modifying it so as to render said bacterium incapable of expressing a protein, preferably an amphenicol-O-acetyltransferase, in particular a functional protein, from said sequence.

This nucleic acid of interest is typically in the form of an expression cassette (or “construct”) such as, for example, a nucleic acid comprising a transcriptional promoter operatively linked (in the sense understood by the person skilled in the art) to one or more (coding) sequences of interest for example an operon comprising several coding sequences of interest whose expression products contribute to the realization of a function of interest within the bacteria, or a nucleic acid comprising in addition an activating sequence and/or a transcription terminator; or in the form of a circular or linear, single- or double-stranded vector, for example a plasmid, a phage, a cosmid, an artificial or synthetic chromosome, comprising one or more expression cassettes as defined above. Preferably, the vector is a plasmid.

The nucleic acids of interest, typically the cassettes or vectors, can be constructed by conventional techniques well known to the skilled person and can comprise one or more promoters, bacterial replication origins (ORI sequences), termination sequences, selection genes, for example antibiotic resistance genes, and sequence(s) (for example “flanking region(s)”) allowing for the targeted insertion of the cassette or vector. Furthermore, these expression cassettes and vectors can be integrated into the genome by techniques well known to the skilled person.

ORI sequences of interest may be selected from pIP404, pAMβ1, pCB102, repH (origin of replication in C. acetobutylicum), ColE1 or rep (origin of replication in E. coli), or any other origin of replication that allows maintenance of the vector, typically the plasmid, within a Clostridium cell.

Termination sequences of interest may be selected from those of the adc, thl, bcs operon, or any other terminator, well known to the skilled person, allowing transcriptional termination within Clostridium.

Selection genes (resistance genes) of interest may be selected from ermB, catP, bla, tetA, tetM, and/or any other gene for resistance to ampicillin, erythromycin, chloramphenicol, thiamphenicol, tetracycline, or any other antibiotic that can be used to select bacteria of the genus Clostridium well known to the skilled person.

In a particular embodiment where the recognized enzyme encoding sequence is a sequence conferring chloramphenicol and/or thiamphenicol resistance to the bacterium, the selection gene used is not a chloramphenicol and/or thiamphenicol resistance gene, and is preferably not any of the catB, catQ, catD or catP genes.

In a particular embodiment, the nucleic acid of interest comprises one or more guide RNAs (gRNAs) targeting a sequence (“target sequence”, “targeted sequence” or “recognized sequence”) encoding, controlling the transcription of, or flanking a sequence encoding, an enzyme of interest in particular an amphenicol-O-acetyltransferase, and/or a modification template (also identified in the present text as an “editing template”), for example a template making it possible to eliminate or modify all or part of the target sequence, preferably in order to inhibit or suppress the expression of the target sequence, typically a matrix comprising sequences homologous (corresponding) to the sequences located upstream and downstream of the target sequence as described above, typically sequences (homologous to said sequences located upstream and downstream of the target sequence) each comprising between 10 or 20 base pairs and 1000, 1500 or 2000 base pairs, for example between 100, 200, 300, 400 or 500 base pairs and 1000, 1200, 1300, 1400 or 1500 base pairs, preferably between 100 and 1500 or between 100 and 1000 base pairs, and even more preferably between 500 and 1000 base pairs or between 200 and 800 base pairs.

A nucleic acid of particular interest is in the form of a vector comprising one or more expression cassettes, each cassette encoding at least one guide RNA (gRNA).

A particular genetic tool in accordance with the invention comprises several (at least two) nucleic acids of interest as described above, said nucleic acids of interest being different from each other.

In a particular embodiment, the nucleic acid of interest used as a genetic tool to transform and/or genetically modify a bacterium of interest, typically a bacterium of the genus Clostridium, is a nucleic acid recognizing a sequence encoding, a sequence controlling transcription, or a sequence flanking a sequence encoding an enzyme conferring on the bacterium resistance to one or more antibiotics, and capable of deleting said sequence within the genome of this bacterium or of rendering it non-functional, in particular a nucleic acid which does not exhibit methylation at the levels of the motifs recognized by the Dam- and Dcm-type methyltransferases (prepared from an Escherichia coli bacterium exhibiting the dam- dcm-genotype).

When the bacterium of interest to be transformed and/or genetically modified is a C. beijerinckii bacterium, in particular belonging to one of the subclades DSM 6423, LMG 7814, LMG 7815, NRRL B-593 and NCCB 27006, the nucleic acid of interest used as a genetic tool, for example the plasmid, is a nucleic acid that does not exhibit methylation at the levels of the motifs recognized by the Dam- and Dcm-type methyltransferases, typically a nucleic acid in which the adenosine (“A”) of the GATC motif and/or the second cytosine “C” of the CCWGG motif (W may correspond to an adenosine (“A”) or to a thymine (“T”)) are demethylated.

A nucleic acid not exhibiting methylation of the motifs recognized by Dam- and Dcm-type methyltransferases can typically be prepared from an Escherichia coli bacterium with the dam-dcm-genotype (for example Escherichia coli INV 110, Invitrogen). This same nucleic acid may contain other methylations performed for example by EcoKI type methyltransferases, the latter targeting the adenines (“A”) of the AAC(N6)GTGC and GCAC(N6)GTT motifs (N may correspond to any base).

In a preferred embodiment, the targeted sequence corresponds to a gene encoding an amphenicol-O-acetyltransferase, for example a chloramphenicol-O-acetyltransferase, such as the catB gene, to a sequence controlling the transcription of this gene, or to a sequence flanking this gene.

A nucleic acid of particular interest used as a genetic tool in the context of the invention is for example a vector, preferably a plasmid, for example the plasmid pCas9ind-ΔcatB of sequence SEQ ID NO: 21 or the plasmid pCas9ind-gRNA_catB of sequence SEQ ID NO: 38 described in the experimental part of the present description, in particular a version of said sequence not exhibiting methylation at the motifs recognized by Dam- and Dcm-type methyltransferases.

The present description also relates to the use of a nucleic acid of interest as described in the present text to transform and/or genetically modify a bacterium of interest, in particular a solventogenic bacterium of the genus Clostridium capable in the wild type of producing isopropanol, in particular capable in the wild type of performing IBE fermentation.

A bacterium capable of producing isopropanol in the wild type, in particular capable of performing IBE fermentation in the wild type, can be for example a bacterium selected from a C. beijerinckii bacterium, a C. diolis bacterium, a C. puniceum bacterium, a C. butyricum bacterium, a C. saccharoperbutylacetonicum bacterium, a C. botulinum bacterium, a C. drakei bacterium, a C. scatologenes bacterium, a C. perfringens bacterium, and a C. tunisiense bacterium, preferably a bacterium selected from a C. beijerinckii bacterium, a C. diolis bacterium, a C. puniceum bacterium and a C. saccharoperbutylacetonicum bacterium.

A particularly preferred bacterium (naturally) capable of producing isopropanol in the wild type, in particular capable of performing IBE fermentation in the wild type, is a C. beijerinckii bacterium.

The acetogenic bacteria of interest are bacteria producing acids and/or solvents from CO₂ and H₂. Acetogenic bacteria of the genus Clostridium can be selected for example from C. aceticum, C. thermoaceticum, C. ljungdahlii, C. autoethanogenum, C. difficile, C. scatologenes and C. carboxydivorans.

In a particular embodiment, the bacterium of the genus Clostridium concerned is an “ABE strain”, preferably C. acetobutylicum strain DSM 792 (also referred to as ATCC strain 824 or LMG 5710), or C. beijerinckii strain NCIMB 8052.

In another particular embodiment, the bacterium of the genus Clostridium concerned is an “IBE strain”, typically one of the C. beijerinckii bacteria identified in the present description, for example a C. beijerinckii whose subclade is selected from DSM 6423, LMG 7814, LMG 7815, NRRL B-593, NCCB 27006, or a C. aurantibutyricum DSZM 793 bacterium (Georges et al. 1983), and a subclade of such a C. beijerinckii or C. aurantibutyricum bacterium showing at least 90%, 95%, 96%, 97%, 98% or 99% identity with strain DSM 6423. A particularly preferred C. beijerinckii bacterium, or subclade of C. beijerinckii bacterium, lacks the pNF2 plasmid.

The respective genomes of the LMG 7814, LMG 7815, NRRL B-593 and NCCB 27006 subclades on the one hand, and DSZM 793 on the other hand, show sequence identity percentages of at least 97% with the genome of the DSM 6423 subclade.

The inventors have performed fermentative tests confirming that C. beijerinckii bacteria of subclade DSM 6423, LMG 7815 and NCCB 27006 are capable of producing isopropanol in the wild type (see Table 1).

TABLE 1

| | Concentration (g/L) | | | | | | | | Glucose | |
|---|---|---|---|---|---|---|---|---|---|---|
| | | Acetic | Butyric | | | Isopro- | | | consumed | |
| | Glucose | acid | acid | Ethanol | Acetone | panol | Butanol | solvents | (g/L) | Yield |
| Control | 56.19 | 2.1406 | 0 | — | — | — | — | 0.00 | | |
| DSM 6423_A | 31.70 | 0 | 0 | 0.16 | 0.24 | 3.72 | 6.16 | 10.11 | 24.50 | 0.41 |
| DSM 6423_B | 29.08 | 0 | 0 | 0.18 | 0.23 | 4.33 | 6.94 | 11.50 | 27.12 | 0.42 |
| LMG_7815_A | 27.65 | 0.93 | 0.73 | 0.16 | 0.35 | 3.93 | 7.28 | 11.56 | 28.55 | 0.40 |
| LMG_7815_B | 27.50 | 0.63 | 0.73 | 0.18 | 0.29 | 4.30 | 7.63 | 12.22 | 28.70 | 0.43 |
| NCCB 27006_A | 36.28 | 0.98 | 2.59 | 0.13 | 0.15 | 2.83 | 5.22 | 8.19 | 19.91 | 0.41 |
| NCCB 27006_B | 36.10 | 1.08 | 2.27 | 0.13 | 0.15 | 2.70 | 5.17 | 8.02 | 20.10 | 0.40 |

Summary of glucose fermentation trials using the naturally isopropanol-producing strains C. beijerinckii DSM 6423, LMG 7815 and NCCB 27006.

In a particularly preferred embodiment of the invention, the C. beijerinckii bacterium is the DSM 6423 subclade bacterium.

In still another preferred embodiment of the invention, the C. beijerinckii bacterium is a C. beijerinckii IFP963 ΔcatB ΔpNF2 strain (registered on 20 Feb. 2019 under deposit number LMG P-31277 with the BCCM-LMG Collection, and also identified in the present text as C. beijerinckii DSM 6423 ΔcatB ΔpNF2).

According to a particular embodiment, the bacterium to be transformed, and preferably genetically modified, is a bacterium that has been exposed to a first transformation step and to a first genetic modification step using a nucleic acid or genetic tool in accordance with the invention that has made it possible to delete at least one extrachromosomal DNA molecule (typically at least one plasmid) naturally present within said bacterium in the wild type.

Another aspect described by the inventors relates to a process for transforming, and preferably further genetically modifying, a bacterium of the genus Clostridium using a genetic tool in accordance with the invention, typically using a nucleic acid of interest in accordance with the invention as described above. The process comprises a step of transforming the bacterium by introducing into said bacterium the nucleic acid of interest described in the present text. The process may further comprise a step of obtaining, recovering, selecting or isolating the transformed bacterium, i.e., the bacterium having the desired recombination(s)/modification(s)/optimization(s).

In a particular embodiment, the process for transforming, and preferably genetically modifying, a bacterium of the genus Clostridium involves a genetic modification tool, for example a genetic modification tool selected from a CRISPR a tool based on the use of group II introns (for example the TARGETRON tool or the CLOSTRON tool) and an allelic exchange tool (for example the ACE tool), and comprises a step of transforming the bacterium by introducing into said bacterium a nucleic acid of interest in accordance with the invention as described above.

The present invention is typically advantageously implemented when the genetic modification tool selected for transforming, and preferably genetically modifying, a bacterium of the genus Clostridium, is intended to be used on a bacterium such as C. beijerinckii, carrying in the wild type a gene encoding an enzyme responsible for resistance to one or more antibiotics, and that the implementation of said genetic tool comprises a step of transforming said bacterium with the aid of a nucleic acid permitting the expression of a marker of resistance to an antibiotic to which this bacterium is resistant in the wild type and/or a step of selecting the transformed and/or genetically modified bacteria with the aid of said antibiotic (to which the bacterium is resistant in the wild type).

A modification advantageously achievable by the present invention, for example using a genetic modification tool selected from a CRISPR tool, a tool based on the use of group II introns and an allelic exchange tool, consists in deleting a sequence encoding an enzyme conferring on the bacterium resistance to one or more antibiotics, or in rendering this sequence non-functional. Another modification advantageously achievable through the present invention consists in genetically modifying a bacterium in order to improve its performance, for example its performance in the production of a solvent or a mixture of solvents of interest, said bacterium having previously been modified through the invention to make it sensitive to an antibiotic to which it was resistant in the wild type.

In a preferred embodiment, the process in accordance with the invention is based on the use of (implements) the clustered regularly interspaced short palindromic repeats (CRISPR) technology/genetic tool, in particular the CRISPR/Cas genetic tool (CRISPR-associated protein).

This method is based on the use of an enzyme called a nuclease (typically a Cas-type nuclease in the case of the CRISPR/Cas genetic tool, such as the CRISPR-associated protein 9 (Cas9 protein) from Streptococcus pyogenes), which, guided by an RNA molecule, will make a double-stranded cut in a DNA molecule (target sequence of interest). The sequence of the guide RNA (gRNA) will determine the cutting site of the nuclease, giving it a very high specificity. Since a double-stranded cut within a DNA molecule essential for the survival of the microorganism is in fact lethal for an organism, the survival of the organism will depend on its ability to repair it (see for example Cui & Bikard, 2016). In bacteria of the genus Clostridium, repair of a double-stranded break depends on a homologous recombination mechanism requiring an intact copy of the cleaved sequence. By providing the bacterium with a DNA fragment that allows this repair to occur while modifying the original sequence, it is possible to force the microorganism to integrate the desired changes into its genome.

The present invention can be implemented in a bacterium of the genus Clostridium using a conventional CRISPR/Cas genetic tool using a single plasmid comprising a nuclease, gRNA and repair template as described by Wang et al. (2015). The CRISPR/Cas system contains two distinct essential elements, i.e., i) an endonuclease, in this case the CRISPR-associated nuclease, Cas, and ii) a guide RNA. The guide RNA is in the form of a chimeric RNA that consists of the combination of a bacterial CRISPR RNA (crRNA) and a tracrRNA (trans-activating CRISPR RNA). The gRNA combines the targeting specificity of the crRNA corresponding to the “spacer sequences” that serve as guides for the Cas proteins, and the conformational properties of the crRNA into a single transcript. When the gRNA and the Cas protein are expressed simultaneously in the cell, the target genomic sequence can be permanently modified thanks to a provided repair template. The skilled person can easily define the sequence and structure of the gRNAs according to the chromosomal region or mobile genetic element to be targeted using well known techniques (see for example the paper by DiCarlo et al., 2013).

The introduction into the bacterium of the elements (nucleic acids or gRNA) of the genetic tool is carried out by any method, direct or indirect, known to the skilled person, for example by transformation, conjugation, microinjection, transfection, electroporation, etc., preferably by electroporation (Mermelstein et al., 1993).

The inventors have recently developed and described a genetic tool for modifying bacteria, suitable for bacteria of the genus Clostridium and usable in the context of the present invention, based on the use of two plasmids (see WO2017/064439, Wasels et al., 2017, and FIG. 15 associated with the present description).

In a particular embodiment, the “first” plasmid of this tool allows the expression of the Cas nuclease and a “second” plasmid, specific to the modification to be performed, contains one or more gRNA expression cassettes (typically targeting different regions of the bacterial DNA) as well as a repair template allowing, by a homologous recombination mechanism, the replacement of a portion of the bacterial DNA targeted by Cas by a sequence of interest. The cas gene and/or the gRNA expression cassette(s) are placed under the control of constitutive or inducible, preferably inducible, expression promoters known to the person skilled in the art (for example described in the application WO2017/064439 and incorporated by reference in the present description), and preferably different but inducible by the same inducing agent.

The gRNAs can be natural RNAs, synthetic RNAs or RNAs produced by recombinant techniques. These gRNAs can be prepared by any methods known to the skilled person such as, for example, chemical synthesis, in vivo transcription or amplification techniques. When multiple gRNAs are used, the expression of each gRNA can be controlled by a different promoter. Preferably, the promoter used is the same for all gRNAs. The same promoter may, in a particular embodiment, be used to enable expression of several, for example only some, or in other words all or some, of the gRNAs intended to be expressed.

In another particular embodiment, suitable for use in the context of the present invention, ii) at least one of said “first” and “second” nucleic acids further encodes one or more guide RNAs (gRNAs), or the genetic tool further comprises one or more guide RNAs, each guide RNA comprising a Cas enzyme binding RNA structure and a sequence complementary to the targeted portion of the bacterial DNA and iii) at least one of said “first” and “second” nucleic acids further comprises a sequence encoding an anti-CRISPR protein under the control of an inducible promoter, or the genetic tool further comprises a “third” nucleic acid encoding an anti-CRISPR protein under the control of an inducible promoter, preferably different from the promoters controlling the expression of Cas and/or the RNA(s) and inducible by another inducing agent.

In a preferred embodiment, the anti-CRISPR protein is capable of inhibiting, preferably neutralizing, the action of the nuclease, preferably during the phase of introducing the nucleic acid sequences of the genetic tool into the bacterial strain of interest.

A particular process involving CRISPR technology that may be implemented in the context of the present invention to transform, and typically to genetically modify by homologous recombination, a bacterium of the genus Clostridium, comprises the following steps:

a) introducing into the bacterium a CRISPR genetic tool described by the inventors in the presence of an agent for inducing expression of an anti-CRISPR protein, and

b) culturing the transformed bacterium obtained at the end of step a) on a medium not containing (or under conditions not involving) the agent for inducing the expression of the anti-CRISPR protein, typically allowing the expression of the Cas/gRNA ribonucleoprotein complex.

In a particular embodiment, the process further comprises, during or after step b), a step of inducing the expression of the inducible promoter(s) controlling the expression of Cas and/or of the guide RNA(s) when such promoter(s) are present within the genetic tool, in order to allow the genetic modification of interest of the bacterium once said genetic tool is introduced into said bacterium. The induction is carried out using a substance that allows the expression inhibition linked to the selected inducible promoter to be lifted.

In another particular embodiment, the process comprises an additional step c) of removing the nucleic acid containing the repair matrix (the bacterial cell being then considered “cleared” of said nucleic acid) and/or removing the guide RNA(s) or sequences encoding the guide RNA(s) introduced with the genetic tool in step a).

In still another particular embodiment, the process comprises one or more additional steps, subsequent to step b) or step c), of introducing an n^{th}, for example third, fourth, fifth, etc., nucleic acid containing a repair matrix distinct from that (those) already introduced and one or more guide RNA expression cassettes allowing the integration of the sequence of interest contained in said distinct repair matrix in a targeted zone of the bacterial genome, in the presence of an agent for inducing the expression of the anti-CRISPR protein, each additional step being followed by a step of culturing the bacterium thus transformed on a medium not containing the agent for inducing the expression of the anti-CRISPR protein, typically allowing the expression of the Cas/gRNA ribonucleoprotein complex

In a particular embodiment of the process in accordance with the invention, the bacterium is transformed using a CRISPR tool or process, such as those described above, using (for example encoding) an enzyme responsible for cutting at least one strand of the target sequence of interest, wherein the enzyme is in a particular embodiment a nuclease, preferably a Cas-type nuclease, preferably selected from a Cas9 enzyme and a MAD7 enzyme. Preferably, the target sequence of interest is a sequence, for example the catB gene, encoding an enzyme conferring on the bacterium resistance to one or more antibiotics, preferably to one or more antibiotics belonging to the class of amphenicols, typically an amphenicol-O-acetyltransferase such as a chloramphenicol-O-acetyltransferase, a sequence controlling the transcription of the coding sequence or a sequence flanking said coding sequence.

Examples of Cas9 proteins suitable for use in the present invention include, but are not limited to, Cas9 proteins from S. pyogenes (see SEQ ID NO: 1 of the application WO2017/064439 and NCBI accession number: WP_010922251.1), Streptococcus thermophilus, Streptococcus mutans, Campylobacter jejuni, Pasteurella multocida, Francisella novicida, Neisseria meningitidis, Neisseria lactamica and Legionella pneumophila (see Fonfara et al., 2013; Makarova et al., 2015).

The MAD7 nuclease (the amino acid sequence of which corresponds to SEQ ID NO: 72), also identified as “Cas12” or “Cpf1”, can otherwise be advantageously used in the context of the present invention by combining it with gRNA(s) known to the person skilled in the art that are capable of binding to such a nuclease (see Garcia-Doval et al., 2017 and Stella S. et al., 2017).

According to a particular aspect, the sequence encoding the MAD7 nuclease is a sequence optimized to be readily expressed in Clostridium strains, preferably the sequence SEQ ID NO: 71.

When used, the anti-CRISPR protein is typically an “anti-Cas” protein, i.e., a protein capable of inhibiting or preventing/neutralizing the action of Cas, and/or a protein capable of inhibiting or preventing/neutralizing the action of a CRISPR/Cas system, for example a type II CRISPR/Cas system when the nuclease is a Cas9 nuclease.

Advantageously, the anti-CRISPR protein is an “anti-Cas9” protein, for example selected from AcrIIA1, AcrIIA2, AcrIIA3, AcrIIA4, AcrIIA5, AcrIIC1, AcrIIC2 and AcrIIC3 (Pawluk et al., 2018). Preferably the “anti-Cas9” protein is AcrIIA2 or AcrIIA4. Such a protein is typically capable of very significantly limiting, ideally preventing, the action of Cas9, for example by binding to the Cas9 enzyme.

Another anti-CRISPR protein that can be advantageously used is an “anti-MAD7” protein, for example the AcrVA1 protein (Marino et al., 2018).

Like the targeted DNA portion (“recognized sequence”), the editing/repair template may itself comprise one or more nucleic acid sequences or nucleic acid sequence portions corresponding to natural and/or synthetic, coding and/or non-coding sequences. The template may also comprise one or more “foreign” sequences, i.e., sequences naturally absent from the genome of bacteria belonging to the genus Clostridium or from the genome of particular species of said genus. The matrix can also include a combination of sequences.

The genetic tool used in the present invention allows the repair template to guide the incorporation into the bacterial genome of a nucleic acid of interest, typically a DNA sequence or portion of sequence comprising at least 1 base pair (bp), preferably at least 1, 2, 3, 4, 5, 10, 15, 20, 50, 100, 1,000, 10,000, 100 000 or 1 000 000 bp, typically between 1 bp and 20 kb, for example 2, 3, 4, 5, 6, 7, 8, 9, 10, 11, 12 or 13 kb, or between 1 bp and 10 kb, preferably between 10 bp and 10 kb or between 1 kb and 10 kb, for example between 1 bp and 5 kb, between 2 kb and 5 kb, or between 2.5 or 3 kb and 5 kb.

In a particular embodiment, the expression of the DNA sequence of interest allows the bacterium of the genus Clostridium to ferment (typically simultaneously) several different sugars, for example at least two different sugars typically at least two different sugars among the sugars comprising 5 carbon atoms (such as glucose or mannose) and/or among the sugars comprising 6 carbon atoms (such as xylose, arabinose or fructose), preferably at least three different sugars, selected for example among glucose, xylose and mannose; glucose, arabinose and mannose; and glucose xylose and arabinose.

In another particular embodiment, the DNA sequence of interest encodes at least one product of interest, preferably a product that promotes solvent production by the bacterium of the genus Clostridium, typically at least one protein of interest, for example an enzyme; a membrane protein such as a transporter; a maturation protein for other proteins (chaperone protein); a transcription factor; or a combination thereof.

In another embodiment, the process in accordance with the invention is based on the use of group II introns, and implements for example the CLOSTRON genetic technology/tool or the TARGETRON genetic tool.

The TARGETRON technology relies on the use of a reprogrammable group II intron (based on the Ll.ltrB intron from Lactococcus lactis), capable of integrating the bacterial genome rapidly at a desired locus (Chen et al., 2005, Wang et al., 2013), typically with the aim of inactivating a targeted gene. The mechanisms of recognition of the edited region as well as insertion into the genome by reverse splicing are based on homology between the intron and said region on the one hand, and on the activity of a protein (ltrA) on the other hand.

The CLOSTRON technology is based on a similar approach, supplemented by the addition of a selection marker in the intron sequence (Heap et al., 2007). This marker allows to select the integration of the intron in the genome, and thus facilitates the obtention of the desired mutants. This genetic system also exploits group I introns. Indeed, the selection marker (called a retrotransposition-activated marker, or RAM) is interrupted by such a genetic element, which prevents its expression from the plasmid (a more precise description of the system: Zhong et al.). Splicing of this genetic element occurs before integration into the genome, resulting in a chromosome with an active form of the resistance gene. An optimized version of the system includes FLP/FRT sites upstream and downstream of this gene, allowing the use of FRT recombinase to remove the resistance gene (Heap et al., 2010).

In another embodiment, the process in accordance with the invention is based on the use of an allelic exchange tool, and implements for example the ACE genetic technology/tool.

The ACE technology is based on the use of an auxotrophic mutant (for uracil in C. acetobutylicum ATCC 824 by deletion of the pyrE gene, which also causes resistance to 5-fluoroorotic acid (A-5-FO); Heap et al., 2012). The system uses the allelic exchange mechanism, which is well known to the skilled person. Following transformation with a pseudo-suicide (very low copy) vector, integration of the latter into the bacterial chromosome by a first allelic exchange event is selected through the resistance gene initially present on the plasmid. The integration step can be performed in two different ways, either within the pyrE locus or within another locus.

In the case of integration at the pyrE locus, the pyrE gene is also placed on the plasmid, but not expressed (no functional promoter). The second recombination restores a functional pyrE gene and can then be selected by auxotrophy (minimal medium, not containing uracil). As the non-functional pyrE gene also has a selectable character (sensitivity to A-5-FO), other integrations are then possible on the same model, successively alternating the state of pyrE between functional and non-functional.

In the case of integration at another locus, a genomic zone allowing expression of the counter-selection marker after recombination is targeted (typically, in an operon after another gene, preferably a highly expressed gene). This second recombination is then selected by auxotrophy (minimum medium not containing uracil).

In the described embodiments based on the use of group II introns, and implementing for example the CLOSTRON genetic technology/tool or the TARGETRON genetic tool, or based on the use of an allelic exchange tool, and implementing for example the ACE genetic technology/tool, the targeted sequence is preferably a sequence flanking the sequence encoding an enzyme of interest, preferably, as explained above, an amphenicol-O-acetyltransferase.

Another subject matter of the invention relates to a transformed and/or genetically modified bacterium, typically a bacterium of the genus Clostridium belonging to a species or corresponding to one of the subclades described by the inventors, obtained by means of a process as described by the inventors in the present text, as well as any derived bacterium, clone, mutant or genetically modified version thereof.

A bacterium thus transformed and/or genetically modified typical of the invention is a bacterium no longer expressing an enzyme conferring resistance to one or more antibiotics, in particular a bacterium no longer expressing an amphenicol-O-acetyltransferase, for example a bacterium expressing the catB gene in the wild type, and lacking said catB gene or incapable of expressing said catB gene once it has been transformed and/or genetically modified thanks to the invention. The bacterium thus transformed and/or genetically modified by virtue of the invention is rendered sensitive to an amphenicol, for example to an amphenicol as described in the present text, in particular to chloramphenicol or thiamphenicol.

A particular example of a preferred genetically modified bacterium in accordance with the invention is the bacterium identified in the present description as C. beijerinckii DSM 6423 ΔcatB as registered under deposit number LMG P-31151 with the Belgian Co-ordinated Collections of Micro-organisms (“BCCM”, K. L. Ledeganckstraat 35, B-9000 Gent-Belgium) on 6 Dec. 2018. The description also relates to any derived bacterium, clone, mutant or genetically modified version of said bacterium remaining susceptible to an amphenicol such as thiamphenicol and/or chloramphenicol.

According to a particular embodiment, the transformed and/or genetically modified bacterium in accordance with the invention not expressing an enzyme conferring resistance to one or more antibiotics, in particular an amphenicol-O-acetyltransferase such as chloramphenicol-O-acetyltransferase, for example the C. beijerinckii DSM 6423 ΔcatB bacterium, is still capable of being transformed, and preferably genetically modified. This can be done with a nucleic acid, for example a plasmid as described in the present description, for example in the experimental part. An example of a nucleic acid that may be advantageously used is the plasmid pCas9_{acr} of sequence SEQ ID NO: 23 (described in the experimental part of the present description).

A particular aspect of the invention indeed relates to the use of a genetically modified bacterium in accordance with the invention, preferably the C. beijerinckii DSM 6423 ΔcatB bacterium deposited under the number LMG P-31151 or a genetically modified version thereof, for example using one of the genetic tools or processes described in the present text, to produce, through the expression of the nucleic acid(s) of interest voluntarily introduced into its genome, one or more solvents, preferably at least isopropanol, preferably on an industrial scale.

The invention also relates to a kit comprising (i) a nucleic acid of interest in accordance with the invention, typically a DNA fragment recognizing a sequence encoding, or controlling the transcription of a sequence encoding, an enzyme of interest in a bacterium of the genus Clostridium in particular in a bacterium capable of performing IBE fermentation as described in the present text, and (ii) at least one tool, preferably several tools, selected from the elements of a genetic modification tool for transforming, and typically genetically modifying, a bacterium of the genus Clostridium, in order to produce an improved variant of said bacterium; a nucleic acid as gRNA; a nucleic acid as repair template; at least one primer pair, for example a primer pair as described in the context of the present invention; and an inducer allowing the expression of a protein encoded by said tool, for example a Cas9 or MAD7 type nuclease.

The genetic modification tool for transforming, and typically genetically modifying, a bacterium of the genus Clostridium, can be selected from, for example, a CRISPR tool, a group II intron-based tool and an allelic exchange tool, as explained above.

The kit may further comprise one or more inducers tailored to the selected inducible promoter(s) optionally used within the genetic tool to control expression of the nuclease used and/or one or more guide RNAs.

A particular kit in accordance with the invention allows the expression of a nuclease comprising a tag.

The kits in accordance with the invention can furthermore comprise one or more consumables such as a culture medium, at least one competent bacterium of the genus Clostridium (i.e., conditioned for transformation), at least one gRNA, a nuclease, one or more selection molecules, or an explanatory leaflet.

The description also relates to the use of a kit in accordance with the invention, or of one or more of the elements of this kit, for the implementation of a process of transformation and ideally of genetic modification of a bacterium of the genus Clostridium described in the present text, and/or for the production of solvent(s) or biofuel(s), or mixtures thereof, preferably on an industrial scale, using a bacterium of the genus Clostridium, preferably a bacterium of the genus Clostridium naturally producing isopropanol.

Solvents that can be produced are typically acetone, butanol, ethanol, isopropanol or a mixture thereof, typically an ethanol/isopropanol, butanol/isopropanol, or ethanol/butanol mixture, preferably an isopropanol/butanol mixture.

The use of bacteria transformed in accordance with the invention typically allows the production per year on an industrial scale of at least 100 tons of acetone, at least 100 tons of ethanol, at least 1000 tons of isopropanol, at least 1800 tons of butanol, or at least 40 000 tons of a mixture thereof.

The following examples and figures are intended to more fully illustrate the invention without limiting its scope.

### BRIEF DESCRIPTION OF THE FIGURES

FIG. 1 shows the Classification of 30 solventogenic Clostridium strains, from Poehlein et al., 2017. Note that the subclade C. beijerinckii NRRL B-593 is also identified in the literature as C. beijerinckii DSM 6423.

FIG. 2 shows the pCas9ind-ΔcatB plasmid map.

FIG. 3 shows the pCas9acr plasmid map.

FIG. 4 shows the pEC750S-uppHR plasmid map.

FIG. 5 shows the pEX-A2-gRNA-upp plasmid map.

FIG. 6 shows the pEC750S-Δupp plasmid map.

FIG. 7 shows the pEC750C-Δupp plasmid map.

FIG. 8 shows the pGRNA-pNF2 map.

FIG. 9 shows the PCR amplification of the catB gene in clones derived from the bacterial transformation of C. beijerinckii strain DSM 6423.

Amplification of about 1.5 kb if the strain still has the catB gene, or about 900 bp if this gene is deleted.

FIG. 10 shows the growth of C. beijerinckii strains DSM6423 WT and ΔcatB on 2YTG medium and 2YTG thiamphenicol selective medium.

FIG. 11 shows the induction of the CRISPR/Cas9_{acr} system in transformants of C. beijerinckii strain DSM 6423 containing pCas9_{acr} and a upp-targeting gRNA expression plasmid, with or without a repair template. Legend: Em, erythromycin; Tm, thiamphenicol; aTc, anhydrotetracycline; ND, not diluted.

FIG. 12A shows the modification of the upp locus of C. beijerinckii DSM 6423 via the CRISPR/Cas9 system. FIG. 12A represents the genetic organization of the upp locus: genes, gRNA target site and repair templates, associated with the corresponding homology regions on the genomic DNA. The primer hybridization sites for PCR verification (RH1010 and RH011) are also indicated.

FIG. 12B shows the modification of the upp locus of C. beijerinckii DSM 6423 via the CRISPR/Cas9 system. FIG. 12B shows the amplification of the upp locus using primers RH010 and RH011. An amplification of 1680 bp is expected in the case of a wild-type gene, compared to 1090 bp for a modified upp gene. M, 100 bp-3 kb size marker (Lonza); WT, wild-type strain.

FIG. 13 shows the PCR amplification verifying the presence of the plasmid pCas9_{ind}. in C. beijerinckii strain 6423 ΔcatB.

FIG. 14 shows PCR amplification (≈900 bp) verifying the presence or not of the natural plasmid pNF2 before induction (positive control 1 and 2) and then after induction on medium containing aTc of the CRISPR-Cas9 system.

FIG. 15 shows the genetic tool for bacterial modification, adapted to bacteria of the genus Clostridium, based on the use of two plasmids (see WO2017/064439, Wasels et al., 2017).

FIG. 16 shows the pCas9ind-gRNA_catB plasmid map.

FIG. 17 shows the CRISPR/Cas9 system used for genome editing as a genetic tool to create one or more gRNA-directed double-stranded cut(s) in genomic DNA using the Cas9 nuclease.

gRNA, guide RNA; PAM, Protospacer Adjacent Motif. Figure modified from Jinek et al., 2012.

FIG. 18 shows the homologous recombination repair of a double-stranded break induced by Cas9. PAM, protospacer adjacent motif.

FIG. 19 shows the use of CRISPR/Cas9 in Clostridium.

ermB, erythromycin resistance gene; catP (SEQ ID NO: 70), thiamphenicol/chloramphenicol resistance gene; tetR, gene whose expression product represses transcription from Pcm-tetO2/1; Pcm-2tetO1 and Pcm-tetO2/1, anhydrotetracycline-inducible promoters, “aTc” (Dong et al., 2012); miniPthl, constitutive promoter (Dong et al., 2012).

FIG. 20 shows the pCas9acr plasmid map (SEQ ID NO: 23).

ermB, erythromycin resistance gene; rep, origin of replication in E. coli; repH, origin of replication in C. acetobutylicum; Tthl, thiolase terminator; miniPthl, constitutive promoter (Dong et al., 2012); Pcm-tetO2/1, promoter repressed by the product of tetR and inducible by anhydrotetracycline, “aTc” (Dong et al. 2012); Pbgal, a promoter repressed by the product of lacR and inducible by lactose (Hartman et al. 2011); acrIIA4, a gene encoding the anti-CRISPR protein AcrII14; bgaR, a gene whose expression product represses transcription from Pbgal.

FIG. 21 shows the relative transformation rates of C. acetobutylicum DSM 792 containing pCas9_{ind} (SEQ ID NO: 22) or pCas9_{acr} (SEQ ID N: 23). Frequencies are expressed as the number of transformants obtained per μg of DNA used in transformation, relative to the transformation frequencies of pEC750C (SEQ ID NO: 106), and represent the means of at least two independent experiments.

FIG. 22 shows the induction of the CRISPR/Cas9 system in DSM 792 strain transformants containing pCas9_{acr} and a bdhB-targeting gRNA expression plasmid, with (SEQ ID NO: 79 and SEQ ID NO: 80) or without (SEQ ID NO: 105) repair template. Em, erythromycin; Tm, thiamphenicol; aTc, anhydrotetracycline; ND, not diluted.

FIG. 23A shows the modification of the bdh locus of C. acetobutylicum DSM792 via the CRISPR/Cas9 system. FIG. 23A shows the genetic organization of the bdh locus. Homologies between repair template and genomic DNA are highlighted with light gray parallelograms. The hybridization sites of primers V1 and V2 are also shown.

FIG. 23B shows the modification of the bdh locus of C. acetobutylicum DSM792 via the CRISPR/Cas9 system. FIG. 23B shows the amplification of the bdh locus using primers V1 and V2. M, 2-log size marker (NEB); P, pGRNA-ΔbdhAΔbdhB plasmid; WT, wild-type strain.

FIG. 24 shows the transformation efficiency (in observed colonies per μg of transformed DNA) for 20 μg of pCas9_{ind} plasmid in C. beijerinckii strain DSM6423. Error bars represent the standard error of the mean for a biological triplicate.

FIG. 25 shows the NF3 plasmid map.

FIG. 26 shows the pEC751S plasmid map.

FIG. 27 shows the pNF3S plasmid map.

FIG. 28 shows the pNF3E plasmid map.

FIG. 29 shows the pNF3C plasmid map.

FIG. 30 shows the transformation efficiency (in observed colonies per μg of transformed DNA) of the plasmid pCas9ind in three strains of C. beijerinckii DSM 6423. Error bars are the standard deviation from the mean for a biological duplicate.

FIG. 31 shows the transformation efficiency (in observed colonies per μg of transformed DNA) of plasmid pEC750C in two strains derived from C. beijerinckii DSM 6423. Error bars are the standard deviation from the mean for a biological duplicate.

FIG. 32 shows the transformation efficiency (in observed colonies per μg of transformed DNA) of the plasmids pEC750C, pNF3C, pFW01, and pNF3E in C. beijerinckii strain IFP963 ΔcatB ΔpNF2. Error bars are the standard deviation of the mean for a biological triplicate.

FIG. 33 shows the transformation efficiency (in colonies observed per μg of transformed DNA) of the plasmids pFW01, pNF3E and pNF3S in C. beijerinckii strain NCIMB 8052.

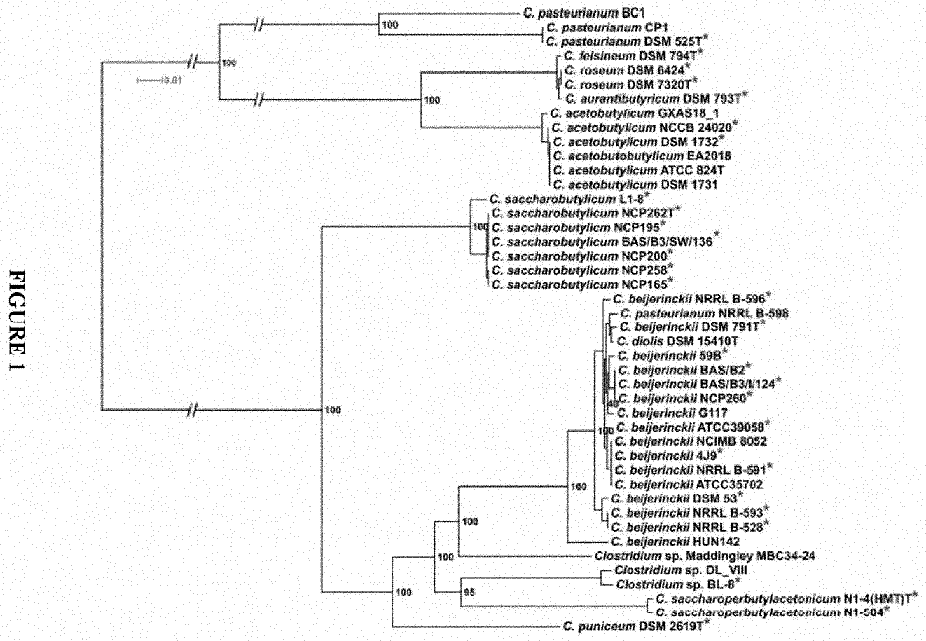

### EXAMPLES

### Example No. 1

### Materials and Methods

### Culture Conditions

C. acetobutylicum DSM 792 was grown in 2YTG medium (Tryptone 16 g·l⁻¹, yeast extract 10 g·l⁻¹, glucose 5 g·l⁻¹, NaCl 4 g·l⁻¹). E. coli NEB10B was grown in LB medium (Tryptone 10 g·l⁻¹, yeast extract 5 g·l⁻¹, NaCl 5 g·l⁻¹). Solid media were made by adding 15 g·l⁻¹ of agarose to liquid media. Erythromycin (at concentrations of 40 or 500 mg·l⁻¹ in 2YTG or LB media, respectively), chloramphenicol (25 or 12.5 mg·l⁻¹ in solid or liquid LB media, respectively), and thiamphenicol (15 mg·l⁻¹ in 2YTG media) were used when necessary.

### Handling of Nucleic Acids

All enzymes and kits were used according to the suppliers' recommendations.

### Construction of Plasmids

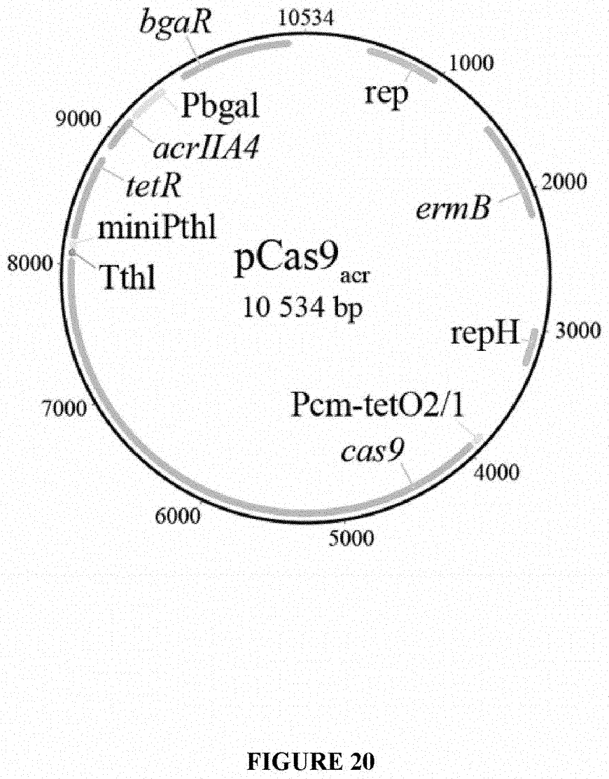

The pCas9_{acr} plasmid (SEQ ID NO: 23), shown in FIG. 20, was constructed by cloning the fragment (SEQ ID NO: 81) containing bgaR and acrIIA4 under the control of the Pbgal promoter synthesized by Eurofins Genomics at the SacI site of the pCas9_{ind} vector (Wasels et al., 2017).

The pGRNA_{ind} plasmid (SEQ ID NO: 82) was constructed by cloning an expression cassette (SEQ ID NO: 83) of a gRNA under the control of the Pcm-2tetO1 promoter (Dong et al., 2012) synthesized by Eurofins Genomics at the SacI site of the pEC750C vector (SEQ ID NO: 106) (Wasels et al., 2017).

The pGRNA-xylB (SEQ ID NO: 102), pGRNA-xylR (SEQ ID NO: 103), pGRNA-glcG (SEQ ID NO: 104) and pGRNA-bdhB (SEQ ID NO: 105) plasmids were constructed by cloning the respective primer pairs 5′-TCATGATTTCTCCATATTAGCTAG-3′ (SEQ ID NO: 84) and 5′-AAACCTAGCTAATATGGAGAAATC-3′ (SEQ ID NO: 85), 5′-TCATGTTACACTTGGAACAGGCGT-3′ (SEQ ID NO: 86) and 5′-AAACACGCCTGTTCCAAGTGTAAC-3′ (SEQ ID NO: 87), 5′-TCATTTCCGGCAGTAGGATCCCCA-3′ (SEQ ID NO: 88) and 5′-AAACTGGGGATCCTACTGCCGGAA-3′ (SEQ ID NO: 89), 5′-TCATGCTTATTACGACATAACACA-3′ (SEQ ID NO: 90) and 5′-AAACTGTGTTATGTCGTAATAAGC-3′ (SEQ ID NO: 91) within the BsaI-digested pGRNA_{ind} plasmid (SEQ ID NO: 82).

The pGRNA-ΔbdhB plasmid (SEQ ID NO: 79) was constructed by cloning the DNA fragment obtained by overlapping PCR assembly of the PCR products obtained with primers 5′-ATGCATGGATCCAAACGAACCCAAAAAGAAAGTTTC-3′ (SEQ ID NO: 92) and 5′-GGTTGATTTCAAATCTGTGTAAACCTACCG-3′ (SEQ ID NO: 93) on the one hand, 5′-ACACAGATTTGAAATCAACCACTTTAACCC-3′ (SEQ ID NO: 94) and 5′-ATGCATGTCGACTCTTAAGAACATGTATAAAGTATGG-3′ (SEQ ID NO: 95) on the other hand, in the pGRNA-bdhB vector digested by BamHI and SacI.

The pGRNA-ΔbdhAΔbdhB plasmid (SEQ ID NO: 80) was constructed by cloning the DNA fragment obtained by overlapping PCR assembly of the PCR products obtained with primers 5′-ATGCATGGATCCAAACGAACCCAAAAAGAAAGTTTC-3′ (SEQ ID NO: 96) and 5′-GCTAAGTTTTAAATCTGTGTAAACCTACCG-3′ (SEQ ID NO: 97) on the one hand, 5′-ACACAGATTTAAAACTTAGCATACTTCTTACC-3′ (SEQ ID NO: 98) and 5′-ATGCATGTCGACCTTCTAATCTCCTCTACTATTTTAG-3′ (SEQ ID NO: 99) on the other hand, in the pGRNA-bdhB vector digested by BamHI and SacI.

### Transformation

C. acetobutylicum DSM 792 was transformed according to the protocol described by Mermelstein et al., 1993. Selection of C. acetobutylicum DSM 792 transformants already containing a Cas9 expression plasmid (pCas9_{ind} or pCas9_{acr}) transformed with a plasmid containing a gRNA expression cassette was performed on solid 2YTG medium containing erythromycin (40 mg·l⁻¹), thiamphenicol (15 mg·l⁻¹) and lactose (40 nM).

### Induction of Cas9 Expression

Induction of cas9 expression was performed via growth of the obtained transformants on solid 2YTG medium containing erythromycin (40 mg·l⁻¹), thiamphenicol (15 mg·l⁻¹) and the cas9 and gRNA expression inducing agent, aTc (1 mg·l⁻¹).

### Amplification of the Bdh Locus

Genome editing of C. acetobutylicum DSM 792 at the bdhA and bdhB gene locus was controlled by PCR using the Q5 High-Fidelity DNA Polymerase enzyme (NEB) with primers V1 (5′-ACACATTGAAGGGAGCTTTT-3′, SEQ ID NO: 100) and V2 (5′-GGCAACAACATCAGGCCTTT-3′, SEQ ID NO: 101).

### Results

### Transformation Efficiency

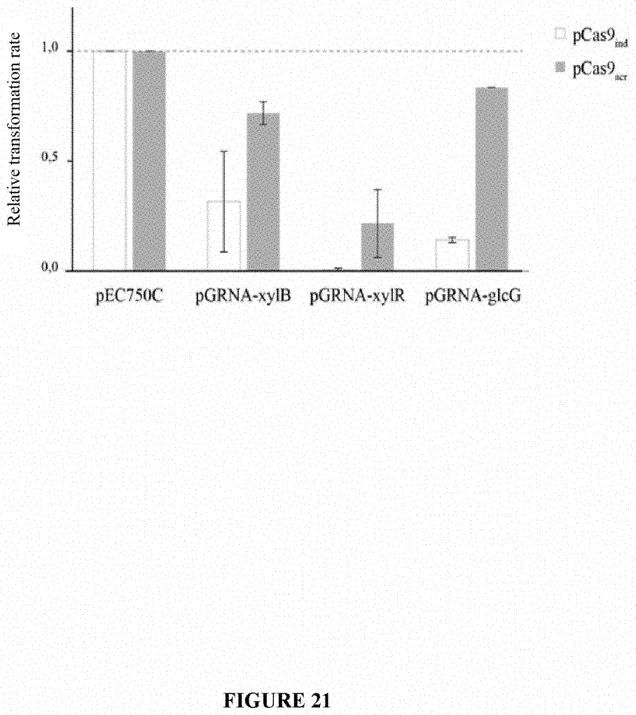

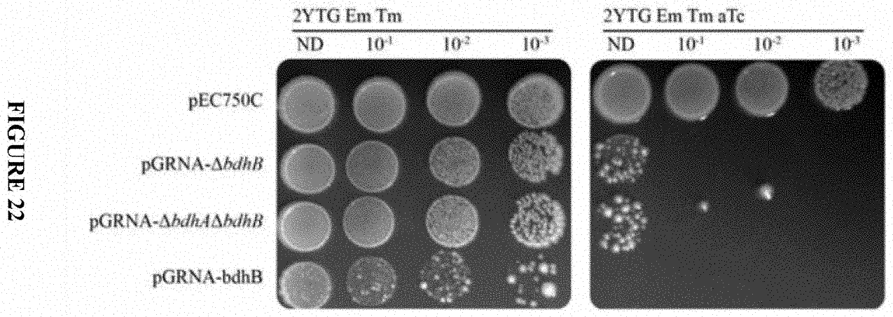

To evaluate the impact of insertion of the acrIIA4 gene on the transformation frequency of the cas9 expression plasmid, different gRNA expression plasmids were transformed into the DSM 792 strain containing pCas9_{ind} (SEQ ID NO: 22) or pCas9_{acr} (SEQ ID NO: 23), and the transformants were selected on lactose-supplemented medium. The transformation frequencies obtained are presented in FIG. 21.

Generation of ΔbdhB and ΔbdhAΔbdhB Mutants

The targeting plasmid containing the gRNA expression cassette targeting bdhB (pGRNA-bdhB—SEQ ID NO: 105) as well as two derived plasmids containing repair templates allowing deletion of the bdhB gene alone (pGRNA-ΔbdhB—SEQ ID NO: 79) or the bdhA and bdhB genes (pGRNA-ΔbdhAΔbdhB—SEQ ID NO: 80) were transformed into the DSM 792 strain containing pCas9_{ind} (SEQ ID NO: 22) or pCas9_{acr} (SEQ ID NO: 23). The transformation frequencies obtained are presented in Table 2:

TABLE 2

| | DSM 792 | | |
|---|---|---|---|
| | pCas9_{ind} | | pCas9_{acr} |
| pEC750C | 32.6 ± 27.1 | cfu · μg⁻¹ | 24.9 ± 27.8 cfu · μg⁻¹ |
| pGRNA-bdhB | 0 | cfu · μg⁻¹ | 17.0 ± 10.7 cfu · μg⁻¹ |
| pGRNA-ΔbdhB | 0 | cfu · μg⁻¹ | 13.3 ± 4.8 cfu · μg⁻¹ |
| pGRNA-ΔbdhAΔbdhB | 0 | cfu · μg⁻¹ | 33.1 ± 13.4 cfu · μg⁻¹ |

Transformation frequencies of strain DSM 792 containing pCas9_{ind} or pCas9_{acr} with plasmids targeting bdhB. Frequencies are expressed as the number of transformants obtained per μg of DNA used in the transformation, and represent the averages of at least two independent experiments.

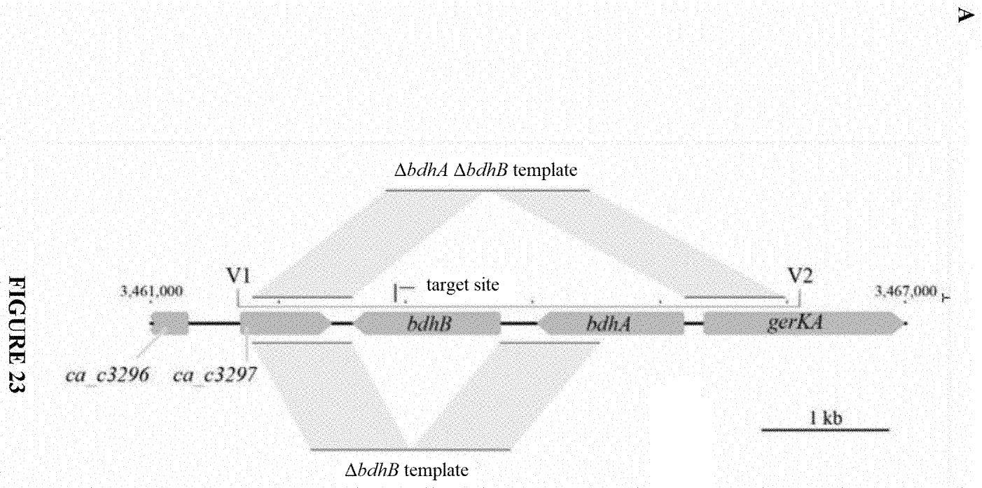

The transformants obtained underwent an induction phase of the expression of the CRISPR/Cas9 system via a passage on medium supplemented with anhydrotetracycline, aTc (FIG. 22).

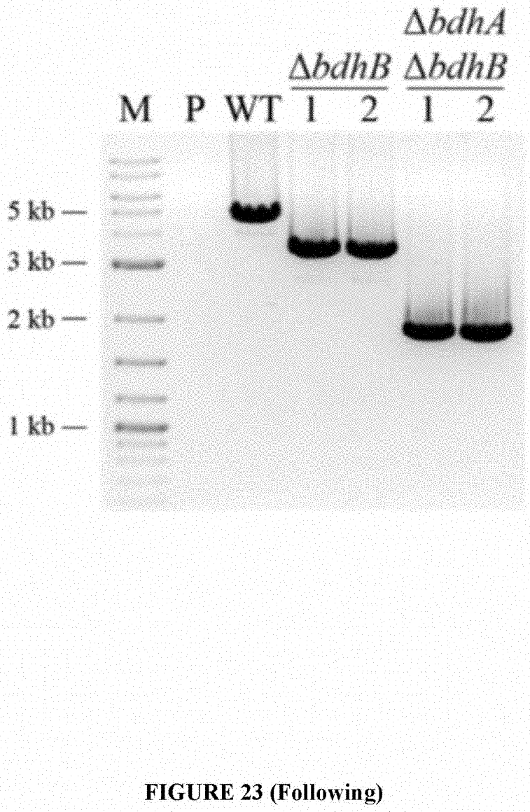

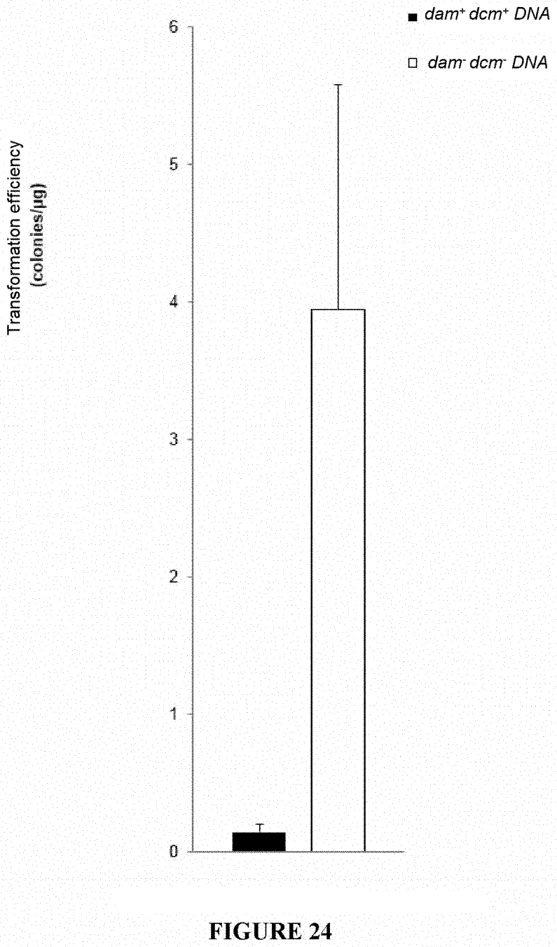

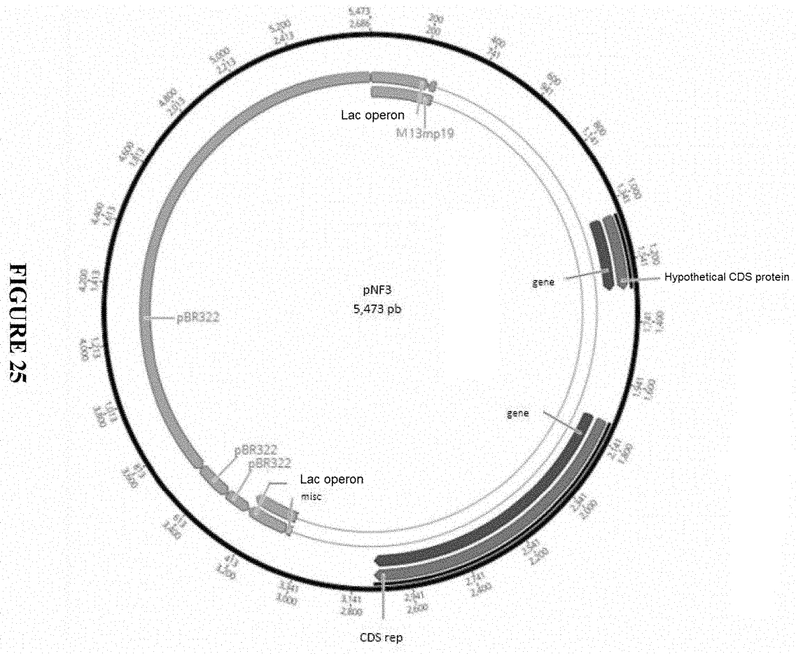

The desired changes were confirmed by PCR on genomic DNA from two aTc-resistant colonies (FIG. 23).

### Conclusions

The CRISPR/Cas9-based genetic tool described in Wasels et al. (2017) uses two plasmids:

the first plasmid, pCas9_{ind}, contains cas9 under the control of an aTc-inducible promoter, and

the second plasmid, derived from pEC750C, contains the gRNA expression cassette (under the control of a second aTc-inducible promoter) and an editing template to repair the double-strand break induced by the system.

However, the inventors observed that some gRNAs still appeared to be too toxic, despite the control of their expression as well as that of Cas9 using aTc-inducible promoters, consequently limiting the efficiency of bacterial transformation by the genetic tool and thus the chromosome modification.

In order to improve this genetic tool, the cas9 expression plasmid was modified by inserting an anti-CRISPR gene, acrIIA4, under the control of a lactose inducible promoter. The transformation efficiencies of different gRNA expression plasmids were thus significantly improved, allowing transformants for all the plasmids tested to be obtained.

It was also possible to perform editing of the bdhB locus within the C. acetobutylicum DSM 792 genome, using plasmids that could not be introduced into the DSM 792 strain containing pCas9_{ind}. The frequencies of modification observed were the same as those observed previously (Wasels et al., 2017), with 100% of the colonies tested modified.

In conclusion, the modification of the cas9 expression plasmid allows a better control of the Cas9-gRNA ribonucleoprotein complex, advantageously facilitating the obtaining of transformants in which the action of Cas9 can be triggered in order to obtain mutants of interest.

### Example No. 2

### Materials and Methods

### Culture Conditions

C. beijerinckii DSM 6423 was grown in 2YTG medium (Tryptone 16 g·L⁻¹, yeast extract 10 g·L⁻¹, glucose 5 g·L⁻¹, NaCl 4 g·L⁻¹). E. coli NEB 10-beta and INV110 were grown in LB medium (Tryptone 10 g·L⁻¹, yeast extract 5 g·L⁻¹, NaCl 5 g·L⁻¹). Solid media were made by adding 15 g·L⁻¹ of agarose to liquid media. Erythromycin (at concentrations of 20 or 500 mg·L⁻¹ in 2YTG or LB media, respectively), chloramphenicol (25 or 12.5 mg·L⁻¹ in solid or liquid LB media, respectively), and thiamphenicol (15 mg·L⁻¹ in 2YTG media) or spectinomycin (at concentrations of 100 or 650 mg·L⁻¹ in LB or 2YTG media, respectively) were used if necessary.

### Nucleic Acids and Plasmid Vectors

All enzymes and kits were used according to the suppliers' recommendations.

Colony PCR tests followed the following protocol:

An isolated colony of C. beijerinckii DSM 6423 is resuspended in 100 μL of 10 mM Tris pH 7.5, 5 mM EDTA. This solution is heated at 98° C. for 10 min without stirring. 0.5 of this bacterial lysate can then be used as a PCR template in 10 μL reactions with Phire (Thermo Scientific), Phusion (Thermo Scientific), Q5 (NEB) or KAPA2G Robust (Sigma-Aldrich) polymerase.

The list of primers used in all the constructs (name/DNA sequence) is detailed below:

ΔcatB_fwd:
(SEQ ID NO: 1)
TGTTATGGATTATAAGCGGCTCGAGGACGTCAAACCATGTTAATCATTGC
ΔcatB_rev:
(SEQ ID NO: 2)
AATCTATCACTGATAGGGACTCGAGCAATTTCACCAAAGAATTCGCTAGC
AcatB_gRNA rev:
(SEQ ID NO: 41)
AATCTATCACTGATAGGGACTCGAGGGGCAAAAGTGTAAAGACAAGCTTC
RH076:
(SEQ ID NO: 3)
CATATAATAAAAGGAAACCTCTTGATCG
RH077:
(SEQ ID NO: 4)
ATTGCCAGCCTAACACTTGG
RH001:
(SEQ ID NO: 5)
ATCTCCATGGACGCGTGACGTCGACATAAGGTACCAGGAATTAGAGCAGC
RH002:
(SEQ ID NO: 6)
TCTATCTCCAGCTCTAGACCATTATTATTCCTCCAAGTTTGCT
RH003:
(SEQ ID NO: 7)
ATAATGGTCTAGAGCTGGAGATAGATTATTTGGTACTAAG
RH004:
(SEQ ID NO: 8)
TATGACCATGATTACGAATTCGAGCTCGAAGCGCTTATTATTGCATTAGC
pEX-fwd:
(SEQ ID NO: 9)
CAGATTGTACTGAGAGTGCACC
pEX-rev:
(SEQ ID NO: 10)
GTGAGCGGATAACAATTTCACAC
pEC750C-fwd:
(SEQ ID NO: 11)
CAATATTCCACAATATTATATTATAAGCTAGC
M13-rev:
(SEQ ID NO: 12)
CAGGAAACAGCTATGAC
RH010:
(SEQ ID NO: 13)
CGGATATTGCATTACCAGTAGC
RH011:
(SEQ ID NO: 14)
TTATCAATCTCTTACACATGGAGC
RH025:
(SEQ ID NO: 15)
TAGTATGCCGCCATTATTACGACA
RH134:
(SEQ ID NO: 16)
GTCGACGTGGAATTGTGAGC
pNF2_fwd:
(SEQ ID NO: 39)
GGGCGCACTTATACACCACC
pNF2_rev:
(SEQ ID NO: 40)
TGCTACGCACCCCCTAAAGG
RH021
(SEQ ID NO: 107)
ACTTGGGTCGACCACGATAAAACAAGGTTTTAAGG
RH022
(SEQ ID NO: 108)
TACCAGGGATCCGTATTAATGTAACTATGATATCAATTCTTG
aad9-fwd2
(SEQ ID NO: 109)
ATGCATGGTCCCAATGAATAGGTTTACACTTACTTTAGTTTTATGG
aad9-rev
(SEQ ID NO: 110)
ATGCGAGTTAACAACTTCTAAAATCTGATTACCAATTAG
RH031
(SEQ ID NO: 111)
ATGCATGGATCCCAATGAATAGGTTTACACTTACTTTAGTTTTATGG
RH032
(SEQ ID NO: 112)
ATGCGAGAGCTCAACTTCTAAAATCTGATTACCAATTAG
RH138
(SEQ ID NO: 113)
ATGCATGGATCCGTCTGACAGTTACCAGGTCC
RH139
(SEQ ID NO: 114)
ATGCGAGAGCTCCAATTGTTCAAAAAAATAATGGCGGAG
RH140
(SEQ ID NO: 115)
ATGCATGGATCCCGGCAGTTTTTCTTTTTCGG
RH141
(SEQ ID NO: 116)
ATGCGAGAGCTCGGTTAAATACTAGTTTTTAGTTACAGAC

The following nine plasmid vectors were prepared:
Plasmid no. 1: pEX-A258-ΔcatB (SEQ ID NO: 17).

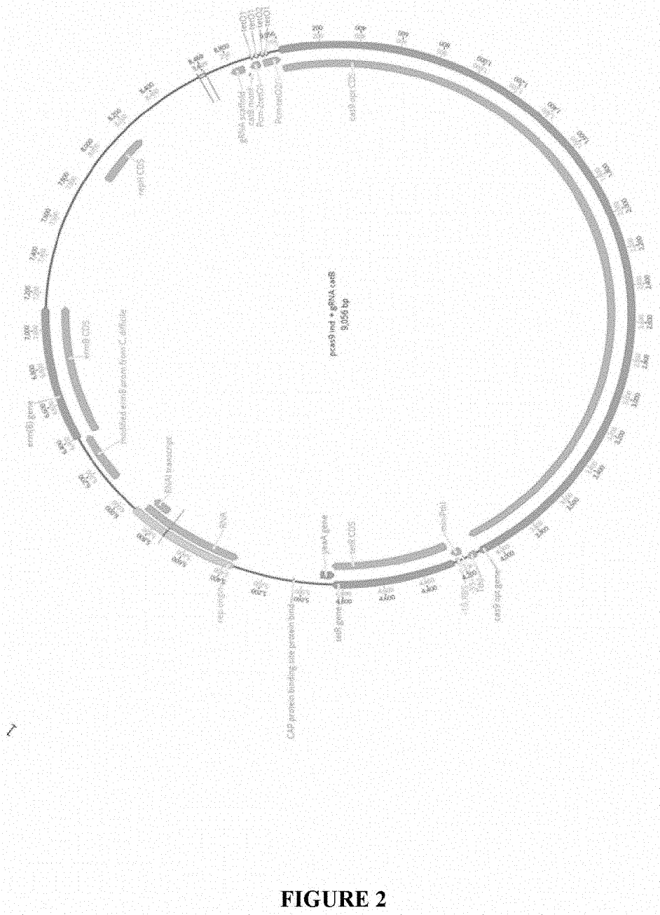

It contains the synthesized DNA fragment ΔcatB cloned into plasmid pEX-A258. This ΔcatB fragment comprises i) a guide RNA expression cassette targeting the catB gene (chloramphenicol resistance gene encoding a chloramphenicol-O-acetyltransferase—SEQ ID NO: 18) of C. beijerinckii DSM6423 under the control of an anhydrotetracycline inducible promoter (expression cassette: SEQ ID NO: 19), and ii) an editing template (SEQ ID NO: 20) comprising 400 homologous bp located upstream and downstream of the catB gene.
Plasmid no. 2: pCas9ind-ΔcatB (see FIG. 2 and SEQ ID NO: 21).

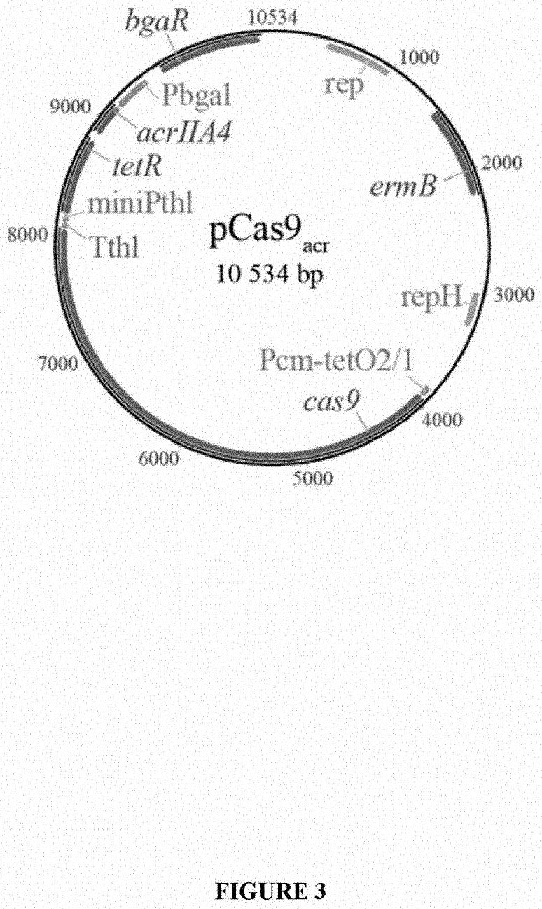

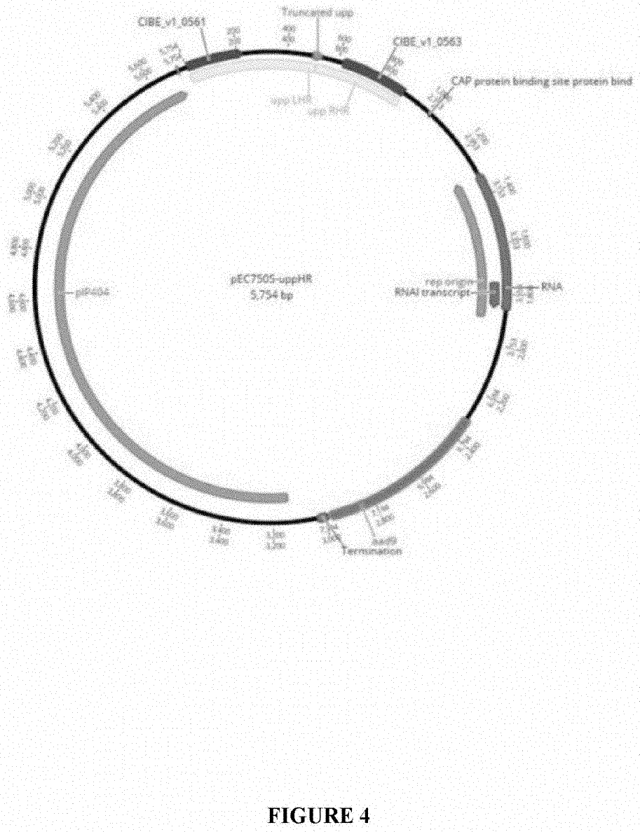

It contains the ΔcatB fragment amplified by PCR (primers ΔcatB_fwd and ΔcatB_rev) and cloned into pCas9ind (described in the patent application WO2017/064439—SEQ ID NO: 22) after digestion of the individual DNAs with the XhoI restriction enzyme.
Plasmid no. 3: pCas9acr (see FIG. 3 and SEQ ID NO: 23).
Plasmid no. 4: pEC750S-uppHR (see FIG. 4 and SEQ ID NO: 24).

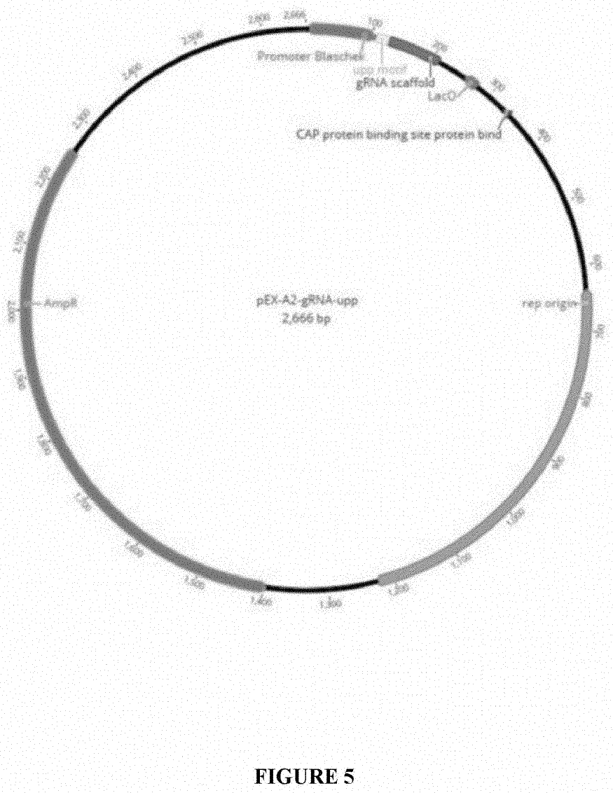

It contains a repair template (SEQ ID NO: 25) used for the deletion of the upp gene and consisting of two homologous DNA fragments upstream and downstream of the upp gene (respective sizes: 500 (SEQ ID NO: 26) and 377 (SEQ ID NO: 27) base pairs). The assembly was obtained using the Gibson cloning system (New England Biolabs, Gibson assembly Master Mix 2X). To this end, the upstream and downstream parts were amplified by PCR from the genomic DNA of strain DSM 6423 (see Maté de Gerando et al., 2018 and accession number PRJEB11626 (see Worldwide Website: ebi.ac.uk/ena/data/view/PRJEB11626)) using the respective primers RH001/RH002 and RH003/RH004. These two fragments were then assembled into the previously linearized pEC750S by restriction enzyme (SalI and SacI restriction enzymes).
Plasmid no. 5: pEX-A2-gRNA-upp (see FIG. 5 and SEQ ID NO: 28).

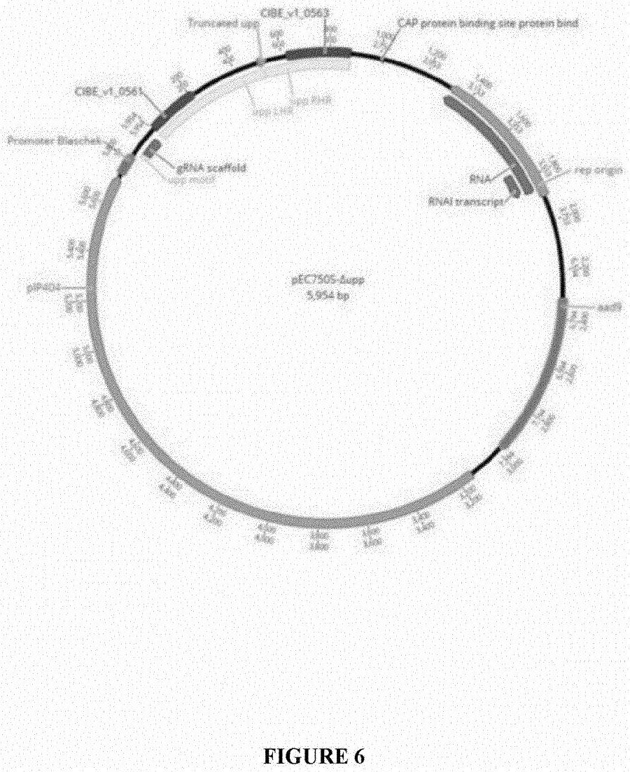

This plasmid comprises the gRNA-upp DNA fragment corresponding to an expression cassette (SEQ ID NO: 29) of a guide RNA targeting the upp gene (upp-targeting protospacer (SEQ ID NO: 31)) under the control of a constitutive promoter (non-coding RNA of sequence SEQ ID NO: 30), inserted into a replication plasmid named pEX-A2.
Plasmid no. 6: pEC750S-Δupp (see FIG. 6 and SEQ ID NO: 32).

It has as a base the plasmid pEC750S-uppHR (SEQ ID NO: 24) and additionally contains the DNA fragment containing a guide RNA expression cassette targeting the upp gene under the control of a constitutive promoter.

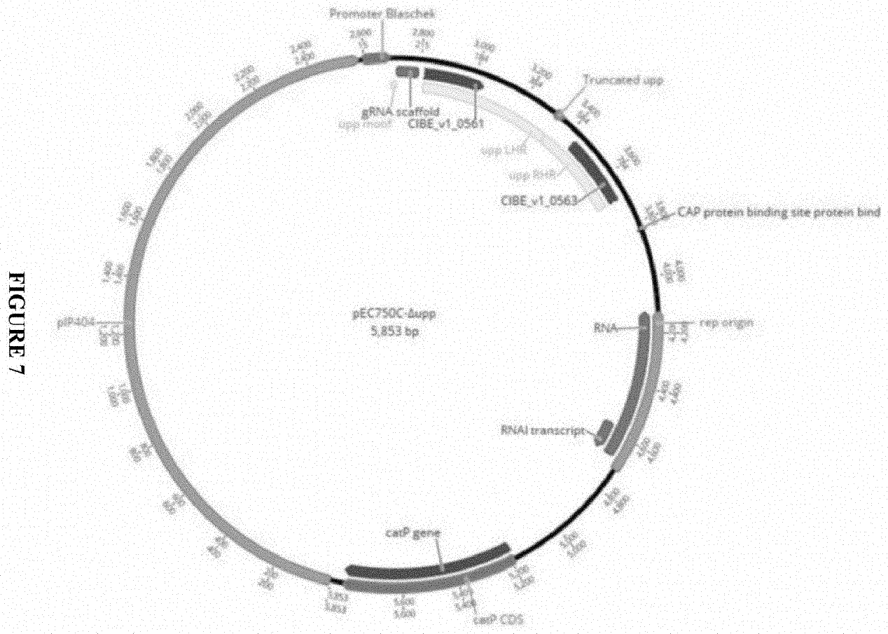

This fragment was inserted into a pEX-A2, designated pEX-A2-gRNA-upp. The insert was then amplified by PCR with the primers pEX-fwd and pEX-rev, and digested with the restriction enzymes XhoI and NcoI. Finally, this fragment was cloned by ligation into pEC750S-uppHR previously digested with the same restriction enzymes to obtain pEC750S-Δupp.
Plasmid no. 7: pEC750C-Δupp (see FIG. 7 and SEQ ID NO: 33).

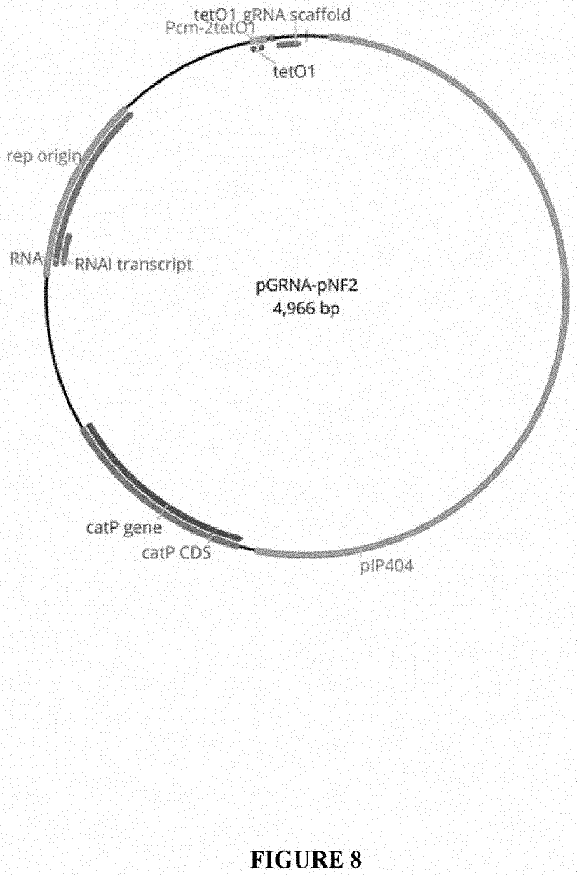

The cassette containing the guide RNA as well as the repair template were then amplified with the primers pEC750C-fwd and M13-rev. The amplicon was digested by restriction enzyme with XhoI and SacI enzymes, and then cloned by enzymatic ligation into pEC750C to obtain pEC750C-Δupp.
Plasmid no. 8: pGRNA-pNF2 (see FIG. 8 and SEQ ID NO: 34).

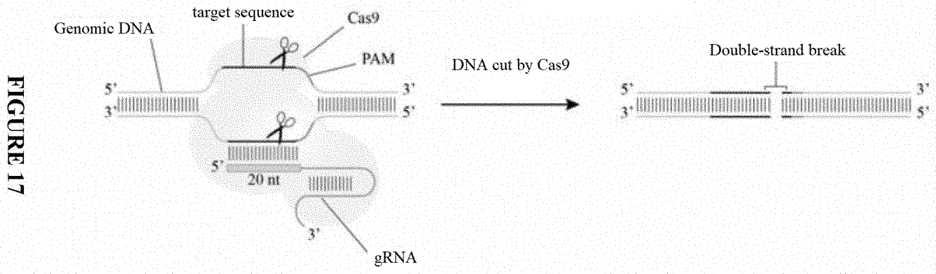

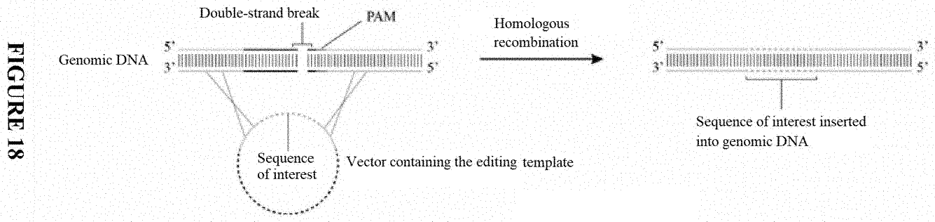

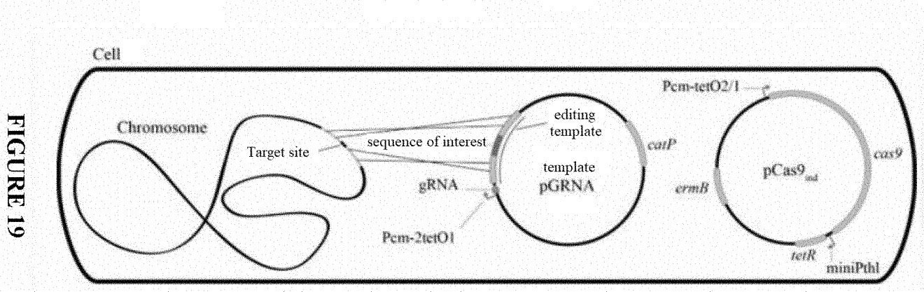

This plasmid has pEC750C as its base and contains a guide RNA expression cassette targeting the pNF2 plasmid (SEQ ID NO: 118).
Plasmid no. 9: pCas9ind-gRNA_catB (see FIG. 16 and SEQ ID NO: 38).

It contains the coding sequence for the guide RNA targeting the catB locus amplified by PCR (primers ΔcatB_fwd and ΔcatB_gRN A_rev) and cloned into pCas9ind (described in the patent application WO2017/064439) after digestion of the individual DNAs with XhoI restriction enzyme and ligation.
Plasmid no. 10: pNF3 (see FIG. 25 and SEQ ID NO: 119).

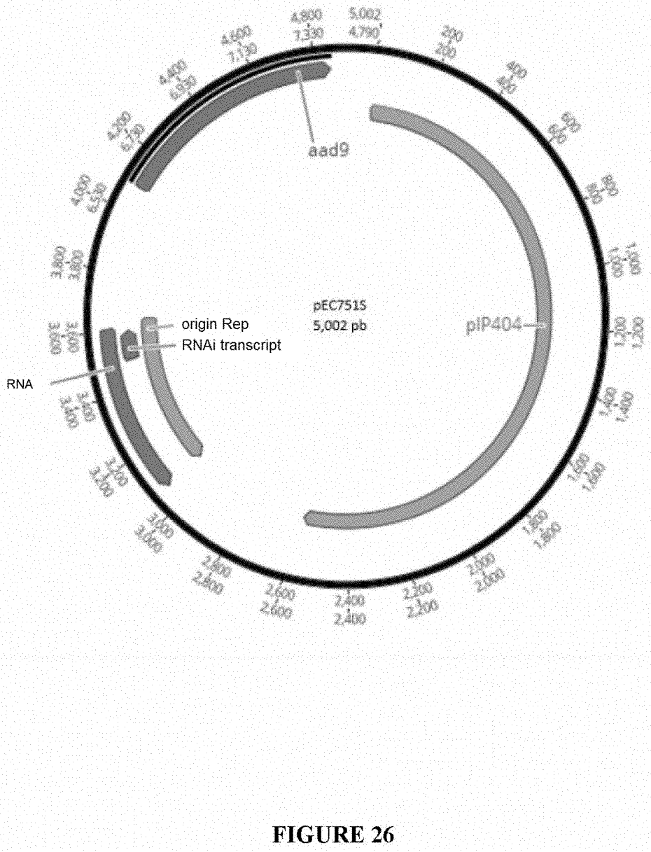

It contains a portion of pNF2, including in particular the origin of replication and a gene encoding a plasmid replication protein (CIBE_p20001), amplified with primers RH021 and RH022. This PCR product was then cloned at the SalI and BamHI restriction sites into plasmid pUC19 (SEQ ID NO: 117).
Plasmid no. 11: pEC751S (see FIG. 26 and SEQ ID NO: 121).

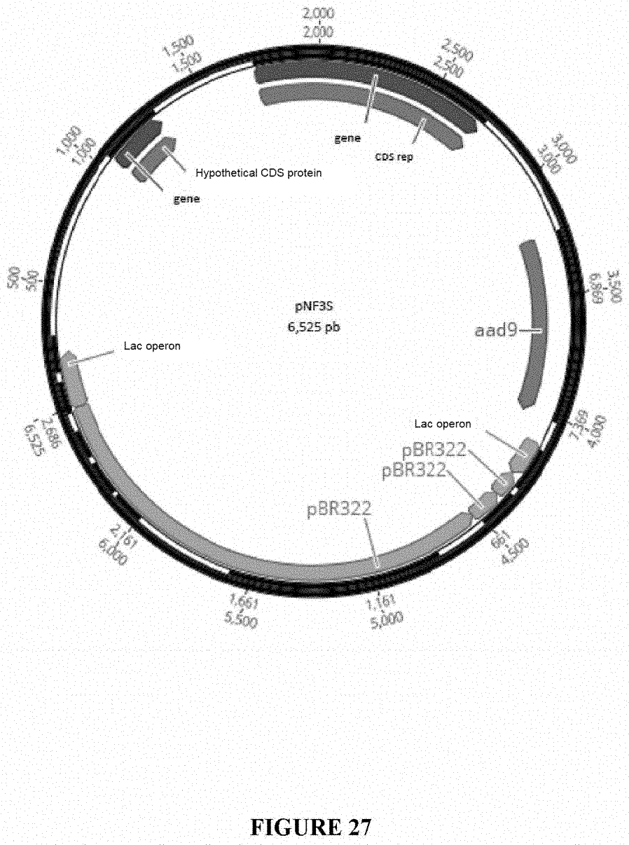

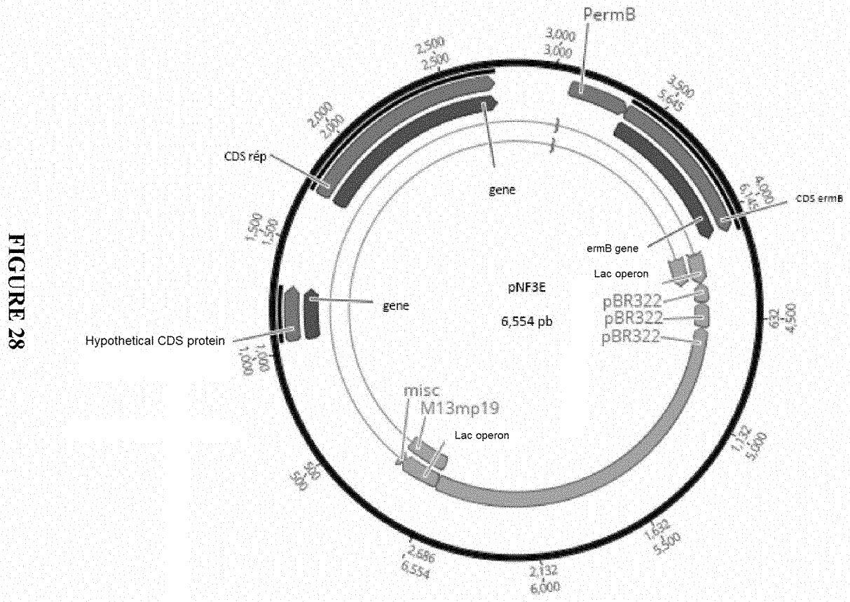

It contains all the elements of pEC750C (SEQ ID NO: 106), except the catP chloramphenicol resistance gene (SEQ ID NO: 70). The latter was replaced by the Enterococcus faecalis aad9 gene (SEQ ID NO: 130), which confers resistance to spectinomycin. This element was amplified with primers aad9-fwd2 and aad9-rev from plasmid pMTL007S-E1 (SEQ ID NO: 120) and cloned into the AvaII and HpaI sites of pEC750C in place of the catP gene (SEQ ID NO: 70).
Plasmid no. 12: pNF3S (see FIG. 27 and SEQ ID NO: 123).

It contains all the elements of pNF3, with an insertion of the aad9 gene (amplified with primers RH031 and RH032 from pEC751S) between the BamHI and SacI sites.
Plasmid no. 13: pNF3E (see FIG. 28 and SEQ ID NO: 124).

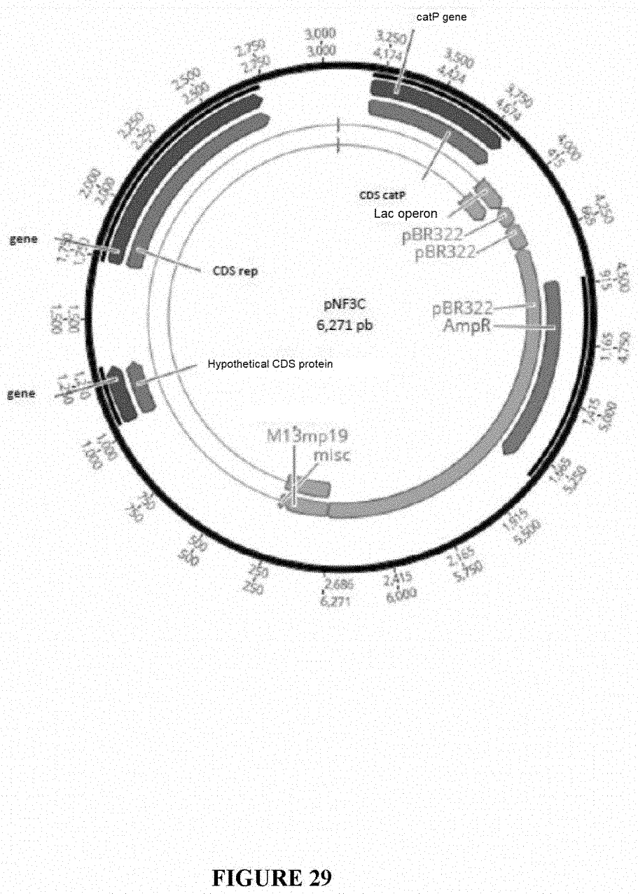

It contains all the elements of pNF3, with an insertion of the Clostridium difficile ermB gene (SEQ ID NO: 131) under the control of the miniPthl promoter. This element was amplified from pFW01 with primers RH138 and RH139 and cloned between the BamHI and SacI sites of pNF3E.
Plasmid no. 14: pNF3C (see FIG. 29 and SEQ ID NO: 125).

It contains all the elements of pNF3, with an insertion of the Clostridium perfringens catP gene (SEQ ID NO: 70). This element was amplified from pEC750C with primers RH140 and RH141 and cloned between the BamHI and SacI sites of pNF3E.

### Results No. 1

Transformation of C. beijerinckii Strain DSM 6423

The plasmids were introduced and replicated into an E. coli dam⁻ dcm⁻ strain (INV110, Invitrogen). This allows the removal of Dam- and Dcm-type methylations on the pCas9ind-ΔcatB plasmid before introducing it by transformation into strain DSM 6423 according to the protocol described by Mermelstein et al. (1993), with the following modifications: the strain is transformed with a larger amount of plasmid (20 μg), at an OD₆₀₀ of 0.8, and using the following electroporation parameters: 100 Ω, 25 μF, 1400 V. Spreading on Petri dish containing erythromycin (20 μg/mL) thus resulted in C. beijerinckii DSM 6423 transformants containing the pCas9ind-ΔcatB plasmid.

Induction of Cas9 Expression and Obtaining C. beijerinckii Strain DSM 6423 ΔcatB

Several erythromycin-resistant colonies were then taken up in 100 μL of culture medium (2YTG) and serially diluted to a dilution factor of 10⁴ in culture medium. For each colony, 8 μL of each dilution was placed on a Petri dish containing erythromycin and anhydrotetracycline (200 ng/mL) to induce expression of the Cas9 nuclease gene.

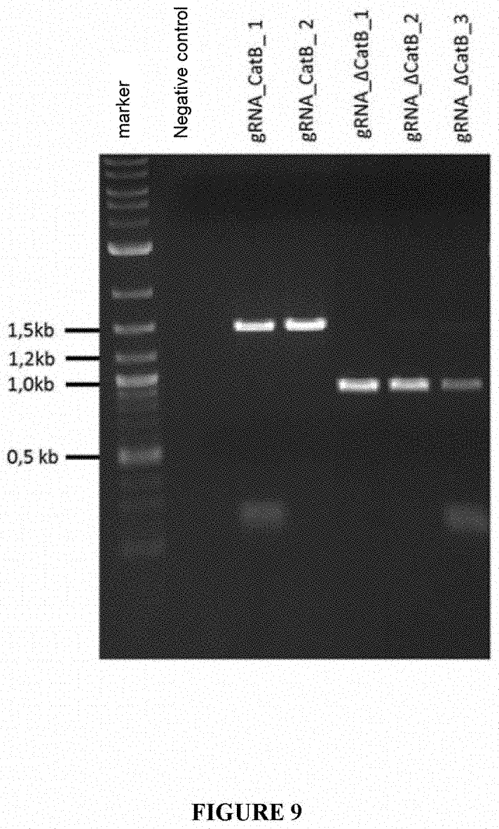

After extraction of genomic DNA, the deletion of the catB gene within the clones grown on this plate was verified by PCR, using primers RH076 and RH077 (see FIG. 9).

Verification of the Sensitivity of C. beijerinckii Strain DSM 6423 ΔcatB to Thiamphenicol

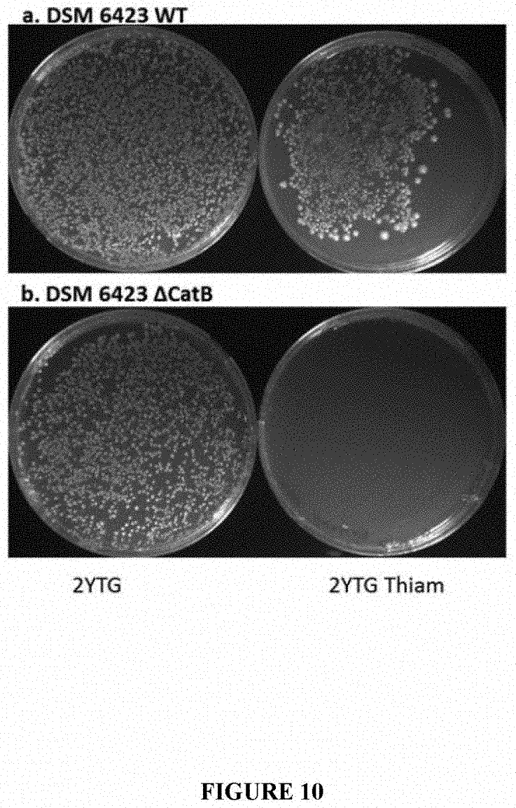

To ensure that the deletion of the catB gene does confer a novel sensitivity to thiamphenicol, comparative analyses on agar medium were performed. Pre-cultures of C. beijerinckii DSM 6423 and C. beijerinckii DSM 6423 ΔcatB were grown on 2YTG medium and then 100 μL of these pre-cultures was plated on 2YTG agar media supplemented or not with thiamphenicol at a concentration of 15 mg/L. FIG. 10 shows that only the initial C. beijerinckii DSM 6423 strain is able to grow on thiamphenicol-supplemented media.

Deletion of the Upp Gene by the CRISPR-Cas9 Tool in C. beijerinckii Strain DSM 6423 ΔcatB

A clone of C. beijerinckii strain DSM 6423 ΔcatB was previously transformed with the pCas9_{acr} vector not exhibiting methylation at the dam- and dcm-type methyltransferase-recognized motifs (prepared from an Escherichia coli bacterium with the dam⁻ dcm⁻ genotype). Verification of the presence of the plasmid pCas9_{acr} maintained in C. beijerinckii strain DSM 6423 was verified by colony PCR with primers RH025 and RH134.

An erythromycin-resistant clone was then transformed with previously demethylated pEC750C-Δupp. The resulting colonies were selected on medium containing erythromycin (20 μg/mL), thiamphenicol (15 μg/mL) and lactose (40 mM).

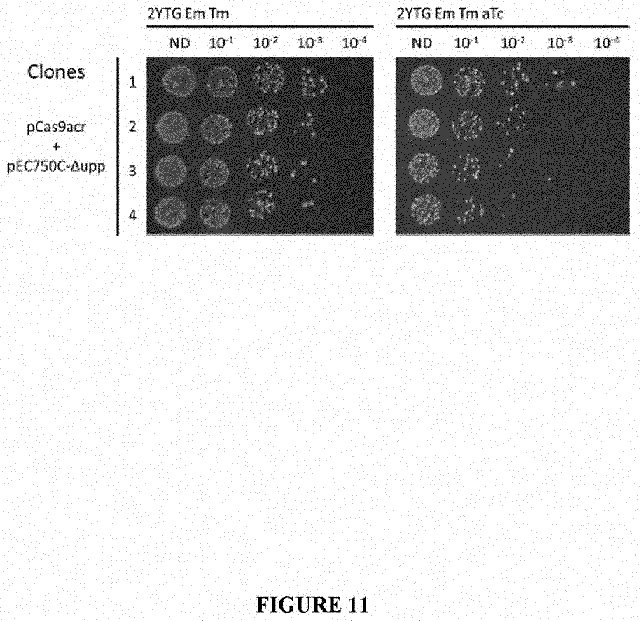

Several of these clones were then resuspended in 100 μL of culture medium (2YTG) and serially diluted in culture medium (to a dilution factor of 10⁴). Five (5) μL of each dilution was placed on a Petri dish containing erythromycin, thiamphenicol and anhydrotetracycline (200 ng/mL) (see FIG. 11).

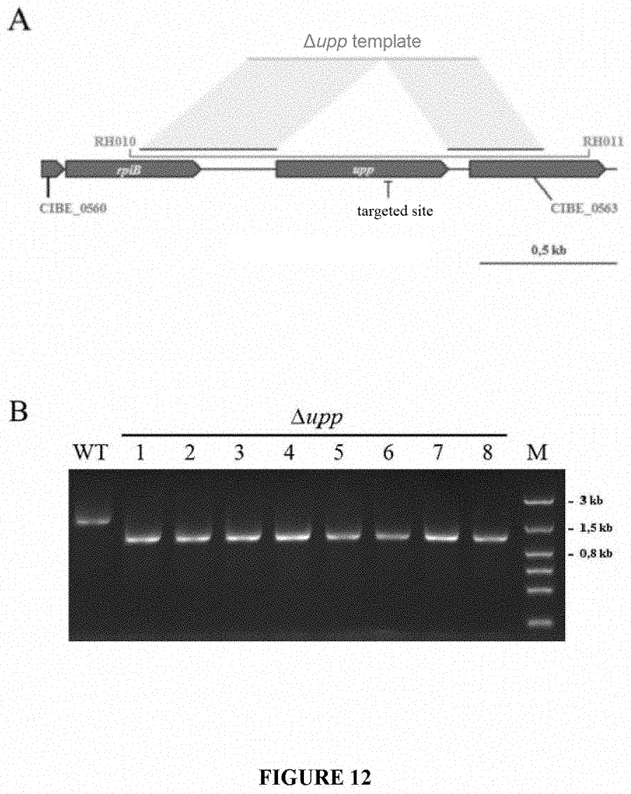

For each clone, two aTc-resistant colonies were tested by PCR colony with primers designed to amplify the upp locus (see FIG. 12).

Deletion of the Natural Plasmid pNF2 by the CRISPR-Cas9 Tool in C. beijerinckii Strain DSM 6423 ΔcatB

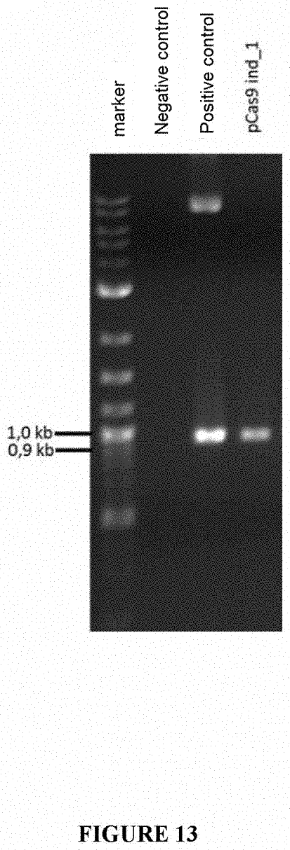

A clone of C. beijerinckii strain DSM 6423 ΔcatB was previously transformed with the pCas9_{ind} vector not exhibiting methylation at the Dam- and Dcm-type methyltransferase-recognized motifs (prepared from an Escherichia coli bacterium with the dam⁻ dcm⁻ genotype). The presence of the plasmid pCas9_{ind} within C. beijerinckii strain DSM6423 was verified by PCR with the primers pCas9_{ind}_fwd (SEQ ID NO: 42) and pCas9_{ind}_rev (SEQ ID NO: 43) (see FIG. 13).

An erythromycin-resistant clone was then used to transform pGRNA-pNF2, prepared from Escherichia coli bacteria with the dam⁻ dcm⁻ genotype.

Several colonies obtained on medium containing erythromycin (20 μg/mL) and thiamphenicol (15 μg/mL) were resuspended in culture medium and serially diluted to a dilution factor of 10⁴. Eight μL of each dilution was placed on a Petri dish containing erythromycin, thiamphenicol and anhydrotetracycline (200 ng/mL) to induce CRISPR/Cas9 expression.

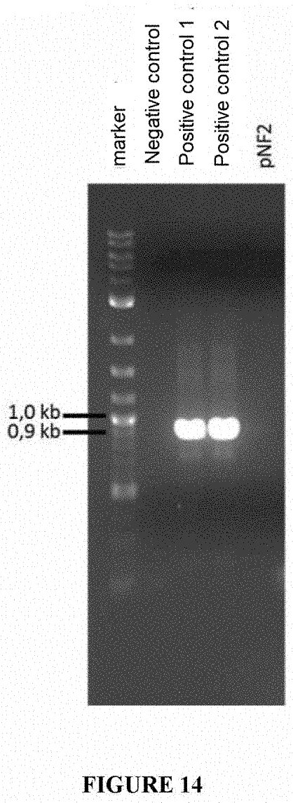

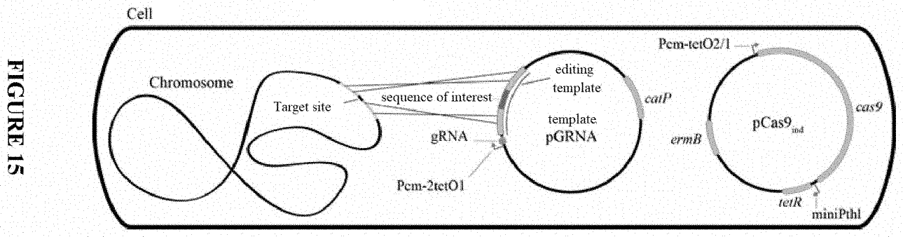

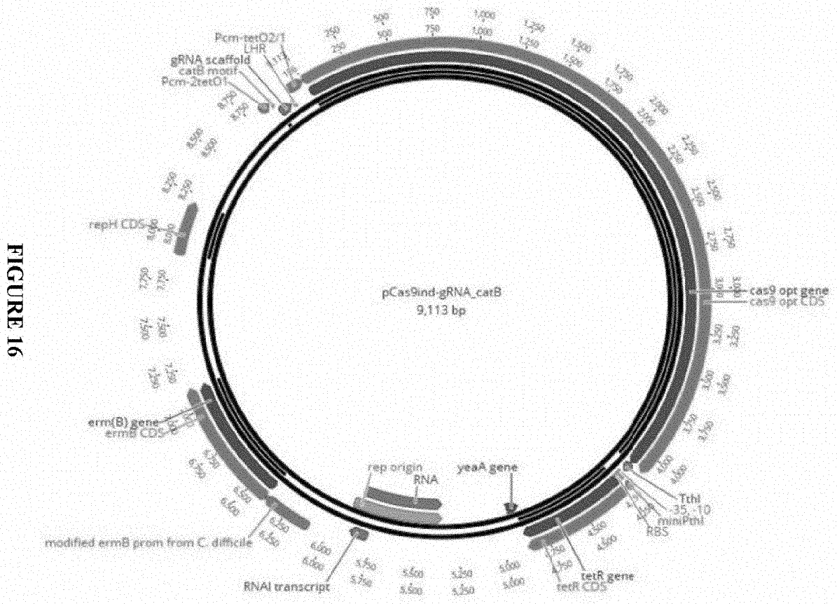

The absence of the natural plasmid Pnf2 was verified by PCR with the primers Pnf2_fwd (SEQ ID NO: 39) and Pnf2_rev (SEQ ID NO: 40) (see FIG. 14).

### Conclusions

In the course of this work, the inventors succeeded in introducing and maintaining different plasmids within Clostridium beijerinckii strain DSM 6423. They succeeded in deleting the catB gene using a CRISPR-Cas9 tool based on the use of a single plasmid. The sensitivity to thiamphenicol of the obtained recombinant strains was confirmed by agar tests.

This deletion allowed them to use the CRISPR-Cas9 tool requiring two plasmids described in the patent application FR1854835. Two examples demonstrating the interest of this application were performed: the deletion of the upp gene and the removal of a non-essential natural plasmid for Clostridium beijerinckii strain DSM 6423.

### Results No. 2

Transformation of C. beijerinckii Strains

The plasmids prepared in the E. coli strain NEB 10-beta are also used to transform the C. beijerinckii strain NCIMB 8052. In contrast, for C. beijerinckii DSM 6423, the plasmids are previously introduced and replicated in an E. coli dam⁻ dcm⁻ strain (INV110, Invitrogen). This allows the removal of Dam- and Dcm-type methylations on the plasmids of interest before introducing them by transformation into strain DSM 6423.

Transformation is otherwise performed similarly for each strain, i.e., according to the protocol described by Mermelstein et al. 1992, with the following modifications: the strain is transformed with a larger amount of plasmid (5-20 μg), at an OD₆₀₀ of 0.6-0.8, and the electroporation parameters are 100 Ω, 25 μF, 1400 V. After 3 h of regeneration in 2YTG, the bacteria are plated on Petri dish (2YTG agar) containing the desired antibiotic (erythromycin: 20-40 μg/mL; thiamphenicol: 15 μg/mL; spectinomycin: 650 μg/mL).

Comparison of Transformation Efficiencies of C. beijerinckii DSM 6423 Strains

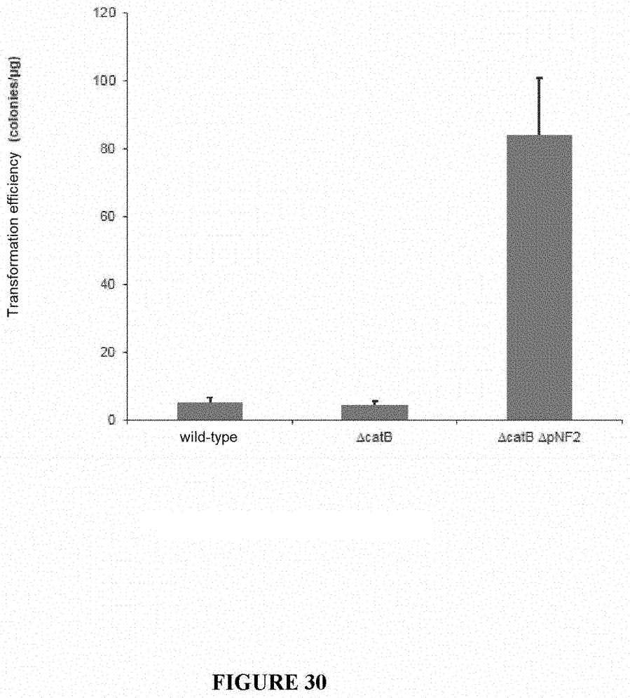

Transformations were performed in biological duplicate in the following C. beijerinckii strains: DSM 6423 wild type, DSM 6423 ΔcatB and DSM 6423 ΔcatB ΔpNF2 (FIG. 30). For this, the pCas9_{ind} vector, notably difficult to use to modify a bacterium because it does not allow good transformation efficiencies, was used. It also contains a gene conferring resistance to erythromycin, an antibiotic to which all three strains are sensitive.

The results indicate an increase in transformation efficiency by a factor of about 15-20 attributable to the loss of the natural plasmid pNF2.

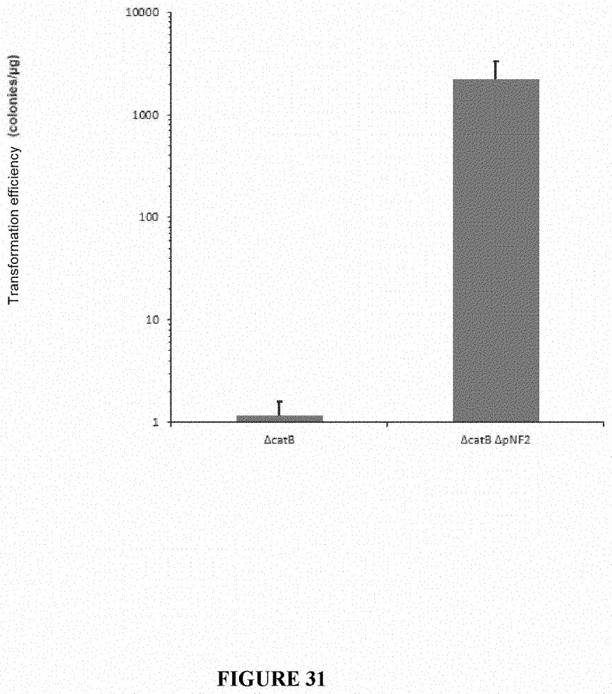

Transformation efficiency was also tested for the plasmid pEC750C, which confers resistance to thiamphenicol, only in DSM 6423 ΔcatB (IFP962 ΔcatB) and DSM 6423 ΔcatB ΔpNF2 (IFP963 ΔcatB ΔpNF2) strains, since the wild-type strain is resistant to this antibiotic (FIG. 31). For this plasmid, the gain in transformation efficiency is even more striking (improvement by a factor of about 2000).

Comparison of Transformation Efficiencies of pNF3 Plasmids with Other Plasmids

To determine the transformation efficiency of plasmids containing the origin of replication of the natural plasmid pNF2, plasmids pNF3E and pNF3C were introduced into the C. beijerinckii strain DSM 6423 ΔcatB ΔpNF2. The use of vectors containing erythromycin or chloramphenicol resistance genes allows comparison of vector transformation efficiency based on the nature of the resistance gene. The plasmids pFW01 and pEC750C were also transformed. These two plasmids contain resistance genes to different antibiotics (erythromycin and thiamphenicol respectively) and are commonly used to transform C. beijerinckii and C. acetobutylicum.

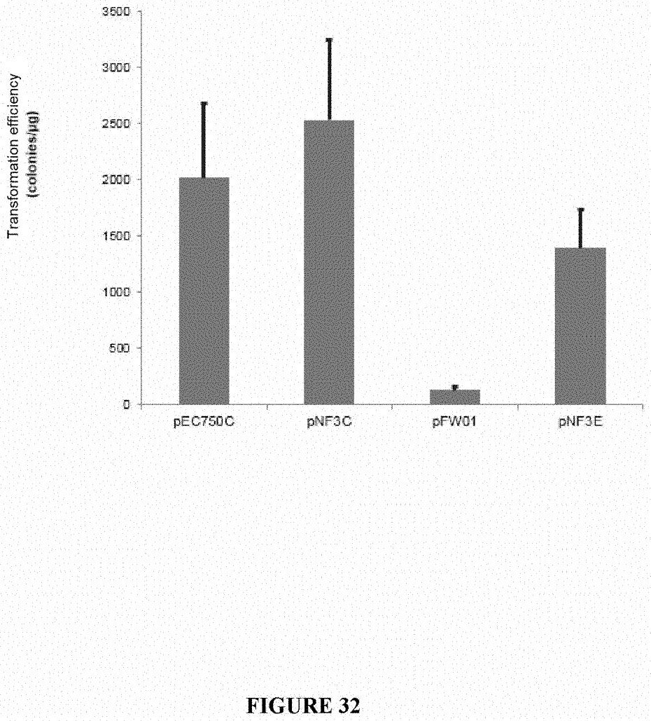

As shown in FIG. 32, the pNF3-based vectors show excellent transformation efficiency, and are particularly usable in C. beijerinckii DSM 6423 ΔcatB ΔpNF2. In particular, pNF3E (which contains an erythromycin resistance gene) shows significantly higher transformation efficiency than pFW01, which comprises the same resistance gene. This same plasmid could not be introduced into the wild type C. beijerinckii DSM 6423 strain (0 colonies obtained with 5 μg of transformed plasmids in biological duplicate), demonstrating the impact of the presence of the natural plasmid pNF2.

Verification of Transformability of pNF3 Plasmids in Other Strains/Species

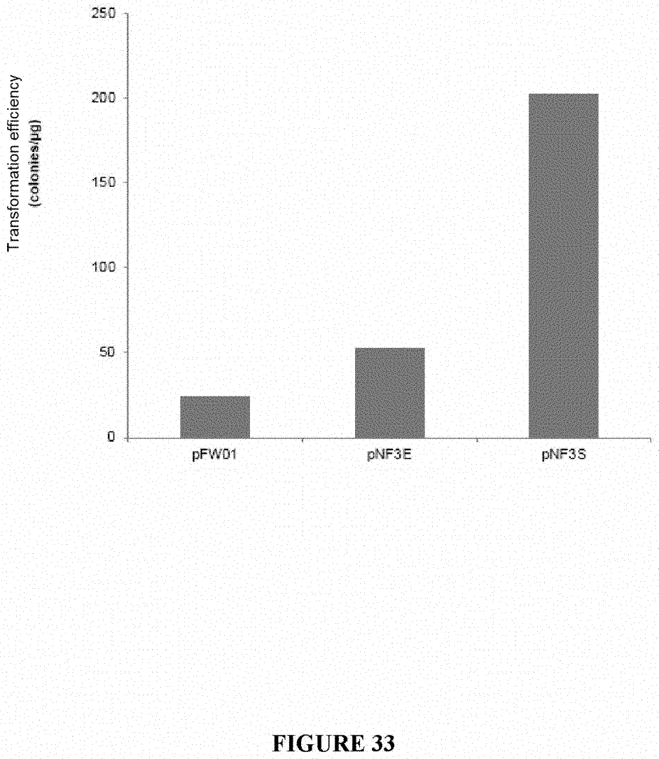

To illustrate the possibility of using this new plasmid in other solventogenic Clostridium strains, the inventors performed a comparative analysis of the transformation efficiencies of the plasmids pFW01, pNF3E, and pNF3S in the ABE strain C. beijerinckii NCIMB 8052 (FIG. 33). Since the NCIMB 8052 strain is naturally resistant to thiamphenicol, pNF3S, conferring resistance to spectinomycin, was used instead of pNF3C.

The results demonstrate that the NCIMB 8052 strain is transformable with the pNF3-based plasmids, proving that these vectors are applicable to C. beijerinckii species in a broad sense.

The applicability of the pNF3-based synthetic vector suite was also tested in the reference strain DSM 792 from C. acetobutylicum. A transformation assay showed the possibility of transforming this strain with the pNF3C plasmid (transformation efficiency of 3 colonies observed per μg of transformed DNA versus 120 colonies/μg for the pEC750C plasmid).

Verification of the Compatibility of pNF3 Plasmids with the Genetic Tool Described in the Application FR18/73492

The patent application FR18/73492 describes the ΔcatB strain as well as the use of a two-plasmid CRISPR/Cas9 system requiring the use of an erythromycin resistance gene and a thiamphenicol resistance gene. To demonstrate the value of the new pNF3 plasmid suite, the pNF3C vector was transformed into the ΔcatB strain already containing the pCas9_{acr} plasmid. The transformation, performed in duplicate, showed a transformation efficiency of 0.625±0.125 colonies/μg DNA (mean±standard error), demonstrating that a pNF3C-based vector can be used in combination with pCas9acr in the ΔcatB strain.

In parallel to these results, a part of the pNF2 plasmid including its origin of replication (SEQ ID NO: 118) could be successfully reused to create a new suite of shuttle vectors (SEQ ID NO: 119, 123, 124 and 125), which can be modified at will, allowing in particular their replication in an E. coli strain as well as their reintroduction in C. beijerinckii DSM 6423. These new vectors present advantageous transformation efficiencies to perform gene editing for example in C. beijerinckii DSM 6423 and derivatives thereof, in particular using the CRISPR/Cas9 tool comprising two different nucleic acids.

These new vectors have also been successfully tested in another strain of C. beijerinckii (NCIMB 8052), and in Clostridium species (in particular C. acetobutylicum), demonstrating their applicability in other organisms of the phylum Firmicutes. A test is also performed on Bacillus.

### Conclusions

These results demonstrate that deletion of the natural plasmid pNF2 significantly increases the transformation frequencies of the bacterium that contained it (by a factor of about 15 for pFW01 and by a factor of about 2000 for pEC750C). This result is particularly interesting in the case of bacteria of the genus Clostridium, known to be difficult to transform, and in particular for the strain C. beijerinckii DSM 6423 which naturally suffers from a low transformation efficiency (less than 5 colonies/μg of plasmid).

### REFERENCES

Banerjee, A., Leang, C., Ueki, T., Nevin, K. P., & Lovley, D. R. (2014). Lactose-inducible system for metabolic engineering of Clostridium ljungdahlii. Applied and environmental microbiology, 80(8), 2410-2416.
Chen J.-S., Hiu S. F. (1986) Acetone-butanol-isopropanol production by Clostridium beijerinckii (synonym, Clostridium butylicum). Biotechnol. Lett. 8:371-376.
Cui, L., & Bikard, D. (2016). Consequences of Cas9 cleavage in the chromosome of Escherichia coli. Nucleic acids research, 44(9), 4243-4251.
Currie, D. H., Herring, C. D., Guss, A. M., Olson, D. G., Hogsett, D. A., & Lynd, L. R. (2013). Functional heterologous expression of an engineered full length CipA from Clostridium thermocellum in Thermoanaerobacterium saccharolyticum. Biotechnology for biofuels, 6(1), 32.
DiCarlo, J. E., Norville, J. E., Mali, P., Rios, X., Aach, J., & Church, G. M. (2013). Genome engineering in Saccharomyces cerevisiae using CRISPR-Cas systems. Nucleic acids research, 41(7), 4336-4343.
Dong, H., Tao, W., Zhang, Y., & Li, Y. (2012). Development of an anhydrotetracycline-inducible gene expression system for solvent-producing Clostridium acetobutylicum: A useful tool for strain engineering. Metabolic engineering, 14(1), 59-67.
Dong, D., Guo, M., Wang, S., Zhu, Y., Wang, S., Xiong, Z., . . . & Huang, Z. (2017). Structural basis of CRISPR-SpyCas9 inhibition by an anti-CRISPR protein. Nature, 546(7658), 436.
Dupuy, B., Mani, N., Katayama, S., & Sonenshein, A. L. (2005). Transcription activation of a UV-inducible Clostridium perfringens bacteriocin gene by a novel σ factor. Molecular microbiology, 55(4), 1196-1206.
Egholm, M., Buchardt, O., Nielsen, P. E., & Berg, R. H. (1992). Peptide nucleic acids (PNA). Oligonucleotide analogs with an achiral peptide backbone. Journal of the American Chemical Society, 114(5), 1895-1897.
Fonfara, I., Le Rhun, A., Chylinski, K., Makarova, K. S., Lecrivain, A. L., Bzdrenga, J., . . . & Charpentier, E. (2013). Phylogeny of Cas9 determines functional exchangeability of dual-RNA and Cas9 among orthologous type II CRISPR-Cas systems. Nucleic acids research, 42(4), 2577-2590.
Garcia-Doval C, Jinek M. Molecular architectures and mechanisms of Class 2 CRISPR-associated nucleases. Curr Opin Struct Biol. 2017 December; 47:157-166. doi: 10.1016/j.sbi.2017.10.015 Ajouter au projet Citavi par DOI. Epub 2017 Nov. 3. Review.
George H. A., Johnson J. L., Moore W. E. C., Holdeman, L. V., Chen J. S. (1983) Acetone, Isopropanol, and Butanol Production by Clostridium beijerinckii (syn. Clostridium butylicum) and Clostridium aurantibutyricum. Appl. Env. Microbiol. 45:1160-1163.
Gonzales y Tucker R D, Frazee B. View from the front lines: an emergency medicine perspective on clostridial infections in injection drug users. Anaerobe. 2014 December; 30:108-15.
Hartman, A. H., Liu, H., & Melville, S. B. (2011). Construction and characterization of a lactose-inducible promoter system for controlled gene expression in Clostridium perfringens. Applied and environmental microbiology, 77(2), 471-478.
Heap, J. T., Ehsaan, M., Cooksley, C. M., Ng, Y. K., Cartman, S. T., Winzer, K., & Minton, N. P. (2012). Integration of DNA into bacterial chromosomes from plasmids without a counter-selection marker. Nucleic acids research, 40(8), e59-e59.
Heap, J. T., Kuehne, S. A., Ehsaan, M., Cartman, S. T., Cooksley, C. M., Scott, J. C., & Minton, N. P. (2010). The ClosTron: mutagenesis in Clostridium refined and streamlined. Journal of microbiological methods, 80(1), 49-55.
Heap, J. T., Pennington, O. J., Cartman, S. T., Carter, G. P., & Minton, N. P. (2007). The ClosTron: a universal gene knock-out system for the genus Clostridium. Journal of microbiological methods, 70(3), 452-464.
Heap, J. T., Pennington, 0. J., Cartman, S. T., & Minton, N. P. (2009). A modular system for Clostridium shuttle plasmids. Journal of microbiological methods, 78(1), 79-85.
Hidalgo-Cantabrana, C., O'Flaherty, S., & Barrangou, R. (2017). CRISPR-based engineering of next-generation lactic acid bacteria. Current opinion in microbiology, 37, 79-87.
Hiu S. F., Zhu C.-X., Yan R.-T., Chen J.-S. (1987) Butanol-ethanol dehydrogenase and butanol-ethanol-isopropanol dehydrogenase: different alcohol dehydrogenases in two strains of Clostridium beijerinckii (Clostridium butylicum). Appl. Env. Microbiol. 53:697-703.
Huang, H., Chai, C., Li, N., Rowe, P., Minton, N. P., Yang, S., & Gu, Y. (2016). CRISPR/Cas9-based efficient genome editing in Clostridium ljungdahlii, an autotrophic gas-fermenting bacterium. ACS synthetic biology, 5(12), 1355-1361.
Huggins, A. S., Bannam, T. L. and Rood, J. I. (1992) Comparative sequence analysis of the catB gene from Clostridium butyricum. Antimicrob. Agents Chemother. 36, 2548-2551.
Ismaiel A. A., Zhu C. X., Colby G. D., Chen, J. S. (1993). Purification and characterization of a primary-secondary alcohol dehydrogenase from two strains of Clostridium beijerinckii. J. Bacteriol. 175:5097-5105.
Jinek, M., Chylinski, K., Fonfara, I., Hauer, M., Doudna, J. A., & Charpentier, E. (2012). A programmable dual-RNA-guided DNA endonuclease in adaptive bacterial immunity. Science, 337(6096), 816-821.
Jones D. T., Woods D. R. (1986) Acetone-butanol fermentation revisited. Microbiological Reviews 50:484-524.
Kolek J., Sedlar K., Provaznik I., Patakova P. (2016). Dam and Dcm methylations prevent gene transfer into Clostridium pasteurianum NRRL B-598: development of methods for electrotransformation, conjugation, and sonoporation. Biotechnol Biofuels. 9:14.
Li, Q., Chen, J., Minton, N. P., Zhang, Y., Wen, Z., Liu, J., . . . & Gu, Y. (2016). CRISPR-based genome editing and expression control systems in Clostridium acetobutylicum and Clostridium beijerinckii. Biotechnology journal, 11(7), 961-972.
Makarova, K. S., Haft, D. H., Barrangou, R., Brouns, S. J., Charpentier, E., Horvath, P., . . . & Van Der Oost, J. (2011). Evolution and classification of the CRISPR-Cas systems. Nature Reviews Microbiology, 9(6), 467.
Makarova, K. S., Wolf, Y. I., Alkhnbashi, O. S., Costa, F., Shah, S. A., Saunders, S. J., . . . & Horvath, P. (2015). An updated evolutionary classification of CRISPR-Cas systems. Nature Reviews Microbiology, 13(11), 722.
Marino, N. D., Zhang, J. Y., Borges, A. L., Sousa, A. A., Leon, L. M., Rauch, B. J., . . . & Bondy-Denomy, J. (2018). Discovery of widespread type I and type V CRISPR-Cas inhibitors. Science, 362(6411), 240-242.
Máté de Gérando, H., Wasels, F., Bisson, A., Clement, B., Bidard, F., Jourdier E., Lopez-Contreras A., Lopes Ferreira N. (2018). Genome and transcriptome of the natural isopropanol producer Clostridium beijerinckii DSM 6423. BMC genomics. 19:242.
Mearls, E. B., Olson, D. G., Herring, C. D., & Lynd, L. R. (2015). Development of a regulatable plasmid-based gene expression system for Clostridium thermocellum. Applied microbiology and biotechnology, 99(18), 7589-7599.
Mermelstein L. D., Welker N. E., Bennett G. N., Papoutsakis E. T. (1993). Expression of cloned homologous fermentative genes in Clostridium acetobutylicum ATCC 824 10:190-195.
Mermelstein L. D., Welker N. E., Bennett G. N., Papoutsakis E. T. (1993). Expression of cloned homologous fermentative genes in Clostridium acetobutylicum ATCC 824 10:190-195.
Moon H G, Jang Y S, Cho C, Lee J, Binkley R, Lee S Y. One hundred years of clostridial butanol fermentation. FEMS Microbiol Lett. 2016 February; 363(3).
Nagaraju, S., Davies, N. K., Walker, D. J. F., Köpke, M., & Simpson, S. D. (2016). Genome editing of Clostridium autoethanogenum using CRISPR/Cas9. Biotechnology for biofuels, 9(1), 219.
Nariya, H., Miyata, S., Kuwahara, T., & Okabe, A. (2011). Development and characterization of a xylose-inducible gene expression system for Clostridium perfringens. Applied and environmental microbiology, 77(23), 8439-8441.
Newcomb, M., Millen, J., Chen, C. Y., & Wu, J. D. (2011). Co-transcription of the celC gene cluster in Clostridium thermocellum. Applied microbiology and biotechnology, 90(2), 625-634.
Pawluk, A., Davidson, A. R., & Maxwell, K. L. (2018). Anti-CRISPR: Discovery, mechanism and function. Nature Reviews Microbiology, 16(1), 12.
Poehlein A., Solano J. D. M., Flitsch S. K., Krabben P., Winzer K., Reid S. J., Jones D. T., Green E., Minton N. P., Daniel R., Dürre P. (2017). Microbial solvent formation revisited by comparative genome analysis. Biotechnol Biofuels. 10:58.
Pyne, M. E., Bruder, M. R., Moo-Young, M., Chung, D. A., & Chou, C. P. (2016). Harnessing heterologous and endogenous CRISPR-Cas machineries for efficient markerless genome editing in Clostridium. Scientific reports, 6.
Rauch, B. J., Silvis, M. R., Hultquist, J. F., Waters, C. S., McGregor, M. J., Krogan, N. J., & Bondy-Denomy, J. (2017). Inhibition of CRISPR-Cas9 with bacteriophage proteins. Cell, 168(1-2), 150-158.
Rajewska M., Wegrzyn K, Konieczny I., FEMS Microbiol Rev. 2012 March; 36(2). AT-rich region and repeated sequences—the essential elements of replication origins of bacterial replicons: 408-34.
Ransom, E. M., Ellermeier, C. D., & Weiss, D. S. (2015). Use of mCherry red fluorescent protein for studies of protein localization and gene expression in Clostridium difficile. Applied and environmental microbiology, 81(5), 1652-1660.
Rogers P., Chen J.-S., Zidwick M. (2006) in The prokaryotes. 3rd edition, Vol. 1, edited by Dworkin M (Springer, New York, USA, 2006). 3rd edition, Vol. 1, pp. 672-755.
Schwarz S, Kehrenberg C, Doublet B, Cloeckaert A. Molecular basis of bacterial resistance to chloramphenicol and florfenicol. FEMS Microbiol Rev. 2004 November; 28(5):519-42.
Stella S, Alcón P, Montoya G. Class 2 CRISPR-Cas RNA-guided endonucleases: Swiss Army knives of genome editing. Nat Struct Mol Biol. 2017 November; 24(11):882-892. doi: 10.1038/nsmb.3486.
Wang, S., Dong, S., Wang, P., Tao, Y., & Wang, Y. (2017). Genome Editing in Clostridium saccharoperbutylacetonicum N1-4 with the CRISPR-Cas9 System. Applied and Environmental Microbiology, 83(10), e00233-17.
Wang Y, Li X, Milne C B, et al. Development of a gene knockout system using mobile group II introns (Targetron) and genetic disruption of acid production pathways in Clostridium beijerinckii. Appl Environ Microbiol. 2013; 79(19): 5853-63.
Wang, Y. et al. Markerless chromosomal gene deletion in Clostridium beijerinckii using CRISPR/Cas9 system. J. Biotechnol. 2015. 200: 1-5.
Wang, Y., Zhang, Z. T., Seo, S. O., Lynn, P., Lu, T., Jin, Y. S., & Blaschek, H. P. (2016). Bacterial genome editing with CRISPR-Cas9: deletion, Integration, single nucleotide modification, and desirable “clean” mutant selection in Clostridium beijerinckii as an example. ACS synthetic biology, 5(7), 721-732.
Wasels, F., Jean-Marie, J., Collas, F., López-Contreras, A. M., & Ferreira, N. L. (2017). A two-plasmid inducible CRISPR/Cas9 genome editing tool for Clostridium acetobutylicum. Journal of microbiological methods. 140:5-11.
Xu, T., Li, Y., Shi, Z., Hemme, C. L., Li, Y., Zhu, Y., . . . & Zhou, J. (2015). Efficient genome editing in Clostridium cellulolyticum via CRISPR-Cas9 nickase. Applied and environmental microbiology, 81(13), 4423-4431.
Yadav, R., Kumar, V., Baweja, M., & Shukla, P. (2018). Gene editing and genetic engineering approaches for advanced probiotics: A Review. Critical reviews in food science and nutrition, 58(10), 1735-1746.
Yue Chen, Bruce A. McClane, Derek J. Fisher, Julian I. Rood, Phalguni Gupta; Construction of an Alpha Toxin Gene Knockout Mutant of Clostridium perfringens Type A by Use of a Mobile Group II Intron; Appl. Environ. Microbiol. November 2005, 71 (11) 7542-7547; DOI: 10.1128/AEM.71.11.7542-7547.2005.
Zhang, J., Liu, Y. J., Cui, G. Z., & Cui, Q. (2015). A novel arabinose-inducible genetic operation system developed for Clostridium cellulolyticum. Biotechnology for biofuels, 8(1), 36.
Zhang C., Tinggang L. Jianzhong H. (2018) Characterization and genome analysis of a butanol-isopropanol-producing Clostridium beijerinckii strain BGS1. Biotechnol Biofuels (2018) 11:280.
Zhong, J., Karberg, M., & Lambowitz, A. M. (2003). Targeted and random bacterial gene disruption using a group II intron (targetron) vector containing a retrotransposition-activated selectable marker. Nucleic acids research, 31(6), 1656-1664.

1-15. (canceled)

16. A nucleic acid recognizing the catB gene of sequence SEQ ID NO: 18 or a sequence at least 70% identical thereto within the genome of a bacterium of the genus Clostridium.

17. The nucleic acid according to claim 16, characterized in that said nucleic acid is selected from an expression cassette, a vector, and a plasmid.

18. The nucleic acid according to claim 16, characterized in that the nucleic acid comprises a guide RNA (gRNA) and/or a modification template.

19. The nucleic acid according to claim 16, characterized in that the Clostridium bacterium is a bacterium capable of producing isopropanol in the wild type.

20. The nucleic acid according to claim 16, characterized in that the Clostridium bacterium is a C. beijerinckii bacterium whose subclade is selected from DSM 6423, LMG 7814, LMG 7815, NRRL B-593, NCCB 27006 and a subclade having at least 95% identity with strain DSM6423.

21. The nucleic acid according to claim 17, characterized in that it is the plasmid pCas9ind-ΔcatB of sequence SEQ ID NO: 21 or the plasmid pCas9ind-gRNA_catB of sequence SEQ ID NO: 38.

22. A process for transforming a bacterium of the genus Clostridium by means of a genetic modification tool, characterized in that it comprises a step of transforming the bacterium by introducing into said bacterium a nucleic acid according to claim 16.

23. The process according to claim 22, characterized in that the bacterium is transformed with a CRISPR tool using an enzyme responsible for cutting at least one strand of the target sequence encoding or controlling the transcription of an amphenicol-O-acetyltransferase.

24. The process according to claim 22, characterized in that the bacterium of the genus Clostridium is a C. beijerinckii subclade selected from DSM 6423, LMG 7814, LMG 7815, NRRL B-593, NCCB 27006, and a subclade exhibiting at least 95% identity to strain DSM 642, and in that the nucleic acid does not exhibit methylation at the motifs recognized by Dam- and Dcm-type methyltransferases.

25. The process according to claim 22, characterized in that the bacterium of the genus Clostridium is a C. beijerinckii DSM 6423 bacterium and in that the nucleic acid recognizes the catB gene of sequence SEQ ID NO: 18 or a sequence at least 70% identical thereto within the genome of C. beijerinckii DSM 6423.

26. A genetically modified bacterium of the genus Clostridium obtained by the process according to claim 22.

27. The genetically modified bacterium of the genus Clostridium obtained by the process according to claim 22, wherein the genetically modified bacterium is a Clostridium bacterium capable of producing isopropanol in the wild type.

28. A C. beijerinckii DSM6423 ΔcatB bacterium deposited under the number LMG P-31151.

29. A method for producing a solvent or a mixture of solvents comprising a step of using the genetically modified bacterium according to claim 27 to produce a solvent or a mixture of solvents.

30. The method according to claim 29, wherein the method is performed on an industrial scale.

31. A kit comprising (i) a nucleic acid according to claim 17 and (ii) at least one tool selected from the elements of a genetic modification tool; a nucleic acid as gRNA; a nucleic acid as repair template; at least one primer pair; and an inducer allowing the expression of a protein encoded by said tool.

32. A method for producing a solvent or a mixture of solvents comprising a step of using the C. beijerinckii DSM6423 ΔcatB bacterium deposited under the number LMG P-31151 according to claim 28 to produce a solvent or mixture of solvents.


SEQUENCE LISTING (134 sequences)

SEQ ID NO: 1 - DNA, 50 bp, Artificial Sequence
  Primer deltacatB-fwd
tgttatggattataagcggctcgaggacgtcaaaccatgttaatcattgc

SEQ ID NO: 2 - DNA, 50 bp, Artificial Sequence
  Primer deltacatB-rev
aatctatcactgatagggactcgagcaatttcaccaaagaattcgctagc

SEQ ID NO: 3 - DNA, 28 bp, Artificial Sequence
  Primer RH076
catataataaaaggaaacctcttgatcg

SEQ ID NO: 4 - DNA, 20 bp, Artificial Sequence
  Primer RH077
attgccagcctaacacttgg

SEQ ID NO: 5 - DNA, 50 bp, Artificial Sequence
  Primer RH001
atctccatggacgcgtgacgtcgacataaggtaccaggaattagagcagc

SEQ ID NO: 6 - DNA, 43 bp, Artificial Sequence
  Primer RH002
tctatctccagctctagaccattattattcctccaagtttgct

SEQ ID NO: 7 - DNA, 40 bp, Artificial Sequence
  Primer RH003
ataatggtctagagctggagatagattatttggtactaag

SEQ ID NO: 8 - DNA, 50 bp, Artificial Sequence
  Primer RH004
tatgaccatgattacgaattcgagctcgaagcgcttattattgcattagc

SEQ ID NO: 9 - DNA, 22 bp, Artificial Sequence
  Primer pEX-fwd
cagattgtactgagagtgcacc

SEQ ID NO: 10 - DNA, 23 bp, Artificial Sequence
  Primer pEX-rev
gtgagcggataacaatttcacac

SEQ ID NO: 11 - DNA, 32 bp, Artificial Sequence
  Primer pEC750C-fwd
caatattccacaatattatattataagctagc

SEQ ID NO: 12 - DNA, 17 bp, Artificial Sequence
  Primer M13-rev
caggaaacagctatgac

SEQ ID NO: 13 - DNA, 22 bp, Artificial Sequence
  Primer RH010
cggatattgcattaccagtagc

SEQ ID NO: 14 - DNA, 24 bp, Artificial Sequence
  Primer RH011
ttatcaatctcttacacatggagc

SEQ ID NO: 15 - DNA, 24 bp, Artificial Sequence
  Primer RH025
tagtatgccgccattattacgaca

SEQ ID NO: 16 - DNA, 20 bp, Artificial Sequence
  Primer RH134
gtcgacgtggaattgtgagc

SEQ ID NO: 17 - DNA, 3658 bp, Artificial Sequence
  pEX-A258-deltacatB
ctcgagctgcagcaaaaaaagcaccgactcggtgccactttttcaagttgataacggactagccttattttaacttgctatttctagctctaaaactgtggtctctcttttcgttgatggtggaatgataagggtttgcaccttaatttctcctattgagaaaatcgtctcttctcagacgtcaaaccatgttaatcattgcttttatcaaaaataggatccactctatcattgatagagtttgaaactctatcattgatagagtataatatctttgttcatgtacatcatgctatctgtgagttttagagctagaaatagcaagttaaaataaggctagtccgttatcaacttgaaaaagtggcaccgagtcggtgctttttttgaagcttgtctttacacttttgcccattaatttttgagttccttatttttagggagcttttattatttttatcatgaaaatttcataaaatactcataaactaaggatgtcttcataatcagattagtactccattttcaatccatttaatctgggaatatgatattttaattacgtattatttaagatatattaacgtgtaatataataccccgcaaatattaattatcacatacatatcccccctttattggggcattttttgtacccattattttagtattgtgcagtacttaaataaaaaaatgccgcaaattcatttttattgaataatgcggtatttcttctattctttatttttattactctataaataatgtaatcaagacatgactatctaaatatatgatatcttaattcataattcgggcctcctaaaaattttcgtaattctattttagaaggcttttttccgtgacctagccatttcaatctcctttttacaatgatatttacgctttagtttattatagcacattctgtaataccgaactattcaattttcagagaccattttttattgattcataacttaagaatactacgaattactctaatattttactttttcttatctcttgttattttaacatcggaattactactaatattaatttttatttttccatccgcatttgctccaacatttttttaactatactttccttttgttaataaattatgttattgttgaacaatataagaaaagtgcgtaacattttttattaaaaataattaggtatttctatctgtggggtaccctcgaggtggcagctctagagctagcgaattctttggtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgcgaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtttggcaattggtcgacctcgagggcgcgcccgta

SEQ ID NO: 18 - DNA, 660 bp, Clostridium beijerinckii
atgaattttaatttgatagatattaatcattggagtagaaagccatactttgaacattatttaaacaatgtgaaatgtacttatagtatgactgccaatatagaaataactgatttattgtatgaaattaaacttaaaaatattaaattttatcctacccttatttatatgattgcaactgtggttaataagcataaagaattccgtatttgttttgatcatgaaggtagtttaggatattgggatagcatgaatccaagctatactatttttcataaagaaaacgaaacattttcaagtatttggacggaatataacaaaagttttttacgtttttatagtgattatcttgacgatataaaaaactatggaaatatcatgaagtttactccgaaatcaaatgaacctgacaatacattttctgtatcaagcattccttgggtgagttttacaggatttaacttgaatgtgtataatgaaggaacatatttaattcctatttttactgcaggaaagtatttcaaacaagaaaataaaatatttattcctatatcaatacaagtacatcatgctatctgtgacggttatcatgctagtagatttattaatgaaatgcaagaattagcatttagttttcaagaatggttagaaaataaataa

SEQ ID NO: 19 - DNA, 160 bp, Artificial Sequence
  gRNA expression cassette
actctatcattgatagagtttgaaactctatcattgatagagtataatatctttgttcatgtacatcatgctatctgtgagttttagagctagaaatagcaagttaaaataaggctagtccgttatcaacttgaaaaagtggcaccgagtcggtgctttt

SEQ ID NO: 20 - DNA, 808 bp, Artificial Sequence
  Editing template
gtctttacacttttgcccattaatttttgagttccttatttttagggagcttttattatttttatcatgaaaatttcataaaatactcataaactaaggatgtcttcataatcagattagtactccattttcaatccatttaatctgggaatatgatattttaattacgtattatttaagatatattaacgtgtaatataataccccgcaaatattaattatcacatacatatcccccctttattggggcattttttgtacccattattttagtattgtgcagtacttaaataaaaaaatgccgcaaattcatttttattgaataatgcggtatttcttctattctttatttttattactctataaataatgtaatcaagacatgactatctaaatatatgatatcttaattcataattcgggcctcctaaaaattttcgtaattctattttagaaggcttttttccgtgacctagccatttcaatctcctttttacaatgatatttacgctttagtttattatagcacattctgtaataccgaactattcaattttcagagaccattttttattgattcataacttaagaatactacgaattactctaatattttactttttcttatctcttgttattttaacatcggaattactactaatattaatttttatttttccatccgcatttgctccaacatttttttaactatactttccttttgttaataaattatgttattgttgaacaatataagaaaagtgcgtaacattttttattaaaaataattaggtatttctatctgtgg

SEQ ID NO: 21 - DNA, 9954 bp, Artificial Sequence
  pCas9ind-deltacatB
catggataaaaagtacagtattggtctagacataggaactaactctgttgggtgggctgttataacagatgaatataaagttccatcaaaaaaatttaaagtattaggaaacactgatagacattcaataaaaaaaaacttgataggtgctttattattcgattcaggagagactgctgaagctacacgtttaaaaagaacagctagacgtagatatacaagaagaaaaaataggatatgttatcttcaagaaatttttagtaatgaaatggcaaaagttgatgattcattctttcacagactagaagaaagtttcttagttgaagaagataagaagcatgaaagacaccctatttttggtaatatcgtagatgaagtagcatatcatgagaagtatccaactatctatcatttaagaaagaaattagttgattctacagataaagctgatctgagattaatatatttagctttagctcatatgattaaatttagaggacattttttaatagaaggtgatttaaacccagacaacagcgatgtagataaattatttatccaattagttcaaacttataatcaattattcgaagagaatccaattaatgcaagtggtgtagacgctaaggctatattatcagctagattatcaaaatctagaagattagaaaatctaatagctcaacttcctggagaaaagaaaaatggactttttgggaacctaatagctctctcactcggactaacaccaaattttaaaagcaattttgatcttgctgaagacgcaaagttacaactatcaaaggatacatacgatgatgatttagataatttgttagctcaaataggtgatcaatatgctgatttgtttcttgcagcaaaaaacttaagtgatgcaattttactatcagatatacttagagtaaatacagaaataacaaaggctcctttatcagcaagtatgattaaacgatatgatgagcatcatcaagatttaacattattaaaggcacttgtaagacaacaattaccagaaaaatataaagaaattttctttgatcaatctaaaaatggatatgctggatatatagacggtggagcaagtcaagaagagttttataaatttataaagcctattttagaaaaaatggatggaactgaagaattacttgttaaacttaacagagaagatttacttagaaaacaaagaacttttgataatggttcaattcctcaccaaattcatttaggagaattacatgctatactaagaagacaagaagatttttatccatttcttaaagataatagagaaaaaattgaaaaaattttaacttttagaataccatattatgtaggaccacttgcaaggggaaattcaagatttgcatggatgactagaaaatcagaagaaactataaccccgtggaattttgaagaagtagtagataaaggagctagtgctcaatcatttatagaaagaatgacaaattttgataagaatcttcctaacgaaaaggttttgccaaagcatagccttctttatgagtattttacagtttataatgagcttactaaagtaaaatacgttacagaaggaatgagaaaaccagcatttttgtctggtgaacaaaagaaagcaatagtagacctattatttaaaacaaataggaaggttaccgtaaagcaacttaaagaagattacttcaaaaaaattgaatgctttgatagtgttgaaatatcaggagttgaagatagatttaatgcttcacttggtacatatcacgatctcttaaaaattataaaagataaggattttttagataatgaagaaaatgaagatattcttgaagatatagtattaacattgacactttttgaagatagagaaatgatagaagaaagattaaaaacatatgcacatctttttgatgataaggttatgaagcaacttaaaagaagaagatatacaggttggggacgtttgtcaagaaagctaattaatggtattagagataaacaatcaggaaagactattctcgattttcttaaatcagatggatttgctaatagaaactttatgcaattaattcatgatgattctcttactttcaaagaggatattcaaaaggctcaagtttctggacaaggcgatagcttacacgaacacattgctaaccttgcagggagccccgctatcaaaaaaggaattttacaaacagttaaagttgtagatgaacttgttaaagttatgggaagacacaaacctgagaatatagttatagaaatggccagagaaaatcaaacaacacaaaaaggacaaaaaaattctagagagagaatgaagagaattgaagaaggaataaaagagctaggatcacaaatattaaaagaacatccagttgaaaatactcaattgcaaaatgaaaagttatatttgtattacttacaaaatggaagagatatgtatgttgatcaagaactcgatattaatagattaagtgactatgatgttgatcatattgttcctcaatcatttttaaaagatgattcaatcgataacaaagtattaactagatcagataaaaatagaggaaagtcagataatgtaccatctgaagaagttgttaaaaaaatgaagaactattggagacaacttttaaatgcaaagctaattacacaaagaaaatttgacaatttaacaaaagcagaaagaggaggattaagcgaattagacaaagctggatttataaaaagacaacttgttgagacaagacaaataactaagcatgttgctcaaatacttgattcaagaatgaatacaaaatatgatgaaaatgataaattaatcagagaagtaaaagtaataacattaaagtcaaaattagtatcagatttcagaaaggattttcaattttacaaagttcgtgaaataaataactatcatcatgctcatgatgcatacttaaatgctgttgtaggaactgctcttattaagaaatatcctaaactagaaagcgaatttgtttatggagattataaagtttatgatgtgcgcaaaatgatcgcgaaatccgaacaagaaatcggtaaggctacagcaaaatatttcttttatagtaatataatgaatttttttaagacagaaataactttggctaatggtgaaatcagaaaaagaccacttatcgaaacaaatggagagacaggagaaatagtatgggataaaggaagagattttgctactgttagaaaagtactaagtatgccacaagtaaatatcgtaaagaaaactgaagttcaaactggaggtttctctaaggaatcaattttacctaagagaaattcagataagttaattgcaaggaaaaaagattgggacccaaaaaaatacggtggttttgatagtccaacagttgcctatagtgttcttgtagtagcgaaagttgagaaaggtaagtcaaaaaagttgaaaagcgtaaaagaacttcttggtatcacaattatggaaagatcttcatttgaaaaaaatccaattgactttttagaagctaagggttataaagaagttaaaaaggatttaatcataaaactaccaaagtatagtctatttgaactcgaaaacggaagaaaacgaatgctcgctagcgcaggagaacttcaaaaaggaaatgaacttgcgctgccatcaaagtatgtaaatttcttatatttagcttctcattatgagaaattaaaaggatcaccagaggataatgaacaaaagcaactatttgtagaacaacacaaacattatttagatgaaataatagaacaaatatctgaattttctaaaagagttatacttgccgacgcaaatctagataaggtgctttcagcgtataataaacacagagataaaccaataagagaacaagcagaaaacattatccatctttttacattaactaatcttggtgcaccagctgcatttaagtactttgatacaacaatagatagaaaaagatacacatctactaaagaagtattagacgcaactttaatacatcaatctattacagggctttatgaaacaagaattgatttaagtcaactaggcggagattaagtcgacaaagtattgttaaaaataactctgtagaattataaattagttctacagagttattttttgacccgggtatattgataaaaataataatagtgggtataattaagttgttaggaggttagttagaatgatgtcaagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggctcgaggacgtcaaaccatgttaatcattgcttttatcaaaaataggatccactctatcattgatagagtttgaaactctatcattgatagagtataatatctttgttcatgtacatcatgctatctgtgagttttagagctagaaatagcaagttaaaataaggctagtccgttatcaacttgaaaaagtggcaccgagtcggtgctttttttgaagcttgtctttacacttttgcccattaatttttgagttccttatttttagggagcttttattatttttatcatgaaaatttcataaaatactcataaactaaggatgtcttcataatcagattagtactccattttcaatccatttaatctgggaatatgatattttaattacgtattatttaagatatattaacgtgtaatataataccccgcaaatattaattatcacatacatatcccccctttattggggcattttttgtacccattattttagtattgtgcagtacttaaataaaaaaatgccgcaaattcatttttattgaataatgcggtatttcttctattctttatttttattactctataaataatgtaatcaagacatgactatctaaatatatgatatcttaattcataattcgggcctcctaaaaattttcgtaattctattttagaaggcttttttccgtgacctagccatttcaatctcctttttacaatgatatttacgctttagtttattatagcacattctgtaataccgaactattcaattttcagagaccattttttattgattcataacttaagaatactacgaattactctaatattttactttttcttatctcttgttattttaacatcggaattactactaatattaatttttatttttccatccgcatttgctccaacatttttttaactatactttccttttgttaataaattatgttattgttgaacaatataagaaaagtgcgtaacattttttattaaaaataattaggtatttctatctgtggggtaccctcgaggtggcagctctagagctagcgaattctttggtgaaattgctcgagtccctatcagtgatagattgaaactctatcattgatagagtataatatctttgttcattagagcgataaacttgaatttgagagggaacttc

SEQ ID NO: 22 - DNA, 8874 bp, Artificial Sequence
  pCas9ind
catggataaaaagtacagtattggtctagacataggaactaactctgttgggtgggctgttataacagatgaatataaagttccatcaaaaaaatttaaagtattaggaaacactgatagacattcaataaaaaaaaacttgataggtgctttattattcgattcaggagagactgctgaagctacacgtttaaaaagaacagctagacgtagatatacaagaagaaaaaataggatatgttatcttcaagaaatttttagtaatgaaatggcaaaagttgatgattcattctttcacagactagaagaaagtttcttagttgaagaagataagaagcatgaaagacaccctatttttggtaatatcgtagatgaagtagcatatcatgagaagtatccaactatctatcatttaagaaagaaattagttgattctacagataaagctgatctgagattaatatatttagctttagctcatatgattaaatttagaggacattttttaatagaaggtgatttaaacccagacaacagcgatgtagataaattatttatccaattagttcaaacttataatcaattattcgaagagaatccaattaatgcaagtggtgtagacgctaaggctatattatcagctagattatcaaaatctagaagattagaaaatctaatagctcaacttcctggagaaaagaaaaatggactttttgggaacctaatagctctctcactcggactaacaccaaattttaaaagcaattttgatcttgctgaagacgcaaagttacaactatcaaaggatacatacgatgatgatttagataatttgttagctcaaataggtgatcaatatgctgatttgtttcttgcagcaaaaaacttaagtgatgcaattttactatcagatatacttagagtaaatacagaaataacaaaggctcctttatcagcaagtatgattaaacgatatgatgagcatcatcaagatttaacattattaaaggcacttgtaagacaacaattaccagaaaaatataaagaaattttctttgatcaatctaaaaatggatatgctggatatatagacggtggagcaagtcaagaagagttttataaatttataaagcctattttagaaaaaatggatggaactgaagaattacttgttaaacttaacagagaagatttacttagaaaacaaagaacttttgataatggttcaattcctcaccaaattcatttaggagaattacatgctatactaagaagacaagaagatttttatccatttcttaaagataatagagaaaaaattgaaaaaattttaacttttagaataccatattatgtaggaccacttgcaaggggaaattcaagatttgcatggatgactagaaaatcagaagaaactataaccccgtggaattttgaagaagtagtagataaaggagctagtgctcaatcatttatagaaagaatgacaaattttgataagaatcttcctaacgaaaaggttttgccaaagcatagccttctttatgagtattttacagtttataatgagcttactaaagtaaaatacgttacagaaggaatgagaaaaccagcatttttgtctggtgaacaaaagaaagcaatagtagacctattatttaaaacaaataggaaggttaccgtaaagcaacttaaagaagattacttcaaaaaaattgaatgctttgatagtgttgaaatatcaggagttgaagatagatttaatgcttcacttggtacatatcacgatctcttaaaaattataaaagataaggattttttagataatgaagaaaatgaagatattcttgaagatatagtattaacattgacactttttgaagatagagaaatgatagaagaaagattaaaaacatatgcacatctttttgatgataaggttatgaagcaacttaaaagaagaagatatacaggttggggacgtttgtcaagaaagctaattaatggtattagagataaacaatcaggaaagactattctcgattttcttaaatcagatggatttgctaatagaaactttatgcaattaattcatgatgattctcttactttcaaagaggatattcaaaaggctcaagtttctggacaaggcgatagcttacacgaacacattgctaaccttgcagggagccccgctatcaaaaaaggaattttacaaacagttaaagttgtagatgaacttgttaaagttatgggaagacacaaacctgagaatatagttatagaaatggccagagaaaatcaaacaacacaaaaaggacaaaaaaattctagagagagaatgaagagaattgaagaaggaataaaagagctaggatcacaaatattaaaagaacatccagttgaaaatactcaattgcaaaatgaaaagttatatttgtattacttacaaaatggaagagatatgtatgttgatcaagaactcgatattaatagattaagtgactatgatgttgatcatattgttcctcaatcatttttaaaagatgattcaatcgataacaaagtattaactagatcagataaaaatagaggaaagtcagataatgtaccatctgaagaagttgttaaaaaaatgaagaactattggagacaacttttaaatgcaaagctaattacacaaagaaaatttgacaatttaacaaaagcagaaagaggaggattaagcgaattagacaaagctggatttataaaaagacaacttgttgagacaagacaaataactaagcatgttgctcaaatacttgattcaagaatgaatacaaaatatgatgaaaatgataaattaatcagagaagtaaaagtaataacattaaagtcaaaattagtatcagatttcagaaaggattttcaattttacaaagttcgtgaaataaataactatcatcatgctcatgatgcatacttaaatgctgttgtaggaactgctcttattaagaaatatcctaaactagaaagcgaatttgtttatggagattataaagtttatgatgtgcgcaaaatgatcgcgaaatccgaacaagaaatcggtaaggctacagcaaaatatttcttttatagtaatataatgaatttttttaagacagaaataactttggctaatggtgaaatcagaaaaagaccacttatcgaaacaaatggagagacaggagaaatagtatgggataaaggaagagattttgctactgttagaaaagtactaagtatgccacaagtaaatatcgtaaagaaaactgaagttcaaactggaggtttctctaaggaatcaattttacctaagagaaattcagataagttaattgcaaggaaaaaagattgggacccaaaaaaatacggtggttttgatagtccaacagttgcctatagtgttcttgtagtagcgaaagttgagaaaggtaagtcaaaaaagttgaaaagcgtaaaagaacttcttggtatcacaattatggaaagatcttcatttgaaaaaaatccaattgactttttagaagctaagggttataaagaagttaaaaaggatttaatcataaaactaccaaagtatagtctatttgaactcgaaaacggaagaaaacgaatgctcgctagcgcaggagaacttcaaaaaggaaatgaacttgcgctgccatcaaagtatgtaaatttcttatatttagcttctcattatgagaaattaaaaggatcaccagaggataatgaacaaaagcaactatttgtagaacaacacaaacattatttagatgaaataatagaacaaatatctgaattttctaaaagagttatacttgccgacgcaaatctagataaggtgctttcagcgtataataaacacagagataaaccaataagagaacaagcagaaaacattatccatctttttacattaactaatcttggtgcaccagctgcatttaagtactttgatacaacaatagatagaaaaagatacacatctactaaagaagtattagacgcaactttaatacatcaatctattacagggctttatgaaacaagaattgatttaagtcaactaggcggagattaagtcgacaaagtattgttaaaaataactctgtagaattataaattagttctacagagttattttttgacccgggtatattgataaaaataataatagtgggtataattaagttgttaggaggttagttagaatgatgtcaagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggctcgagtccctatcagtgatagattgaaactctatcattgatagagtataatatctttgttcattagagcgataaacttgaatttgagagggaacttc

SEQ ID NO: 23 - DNA, 10534 bp, Artificial Sequence
  pCas9acr
cgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggctcgagtccctatcagtgatagattgaaactctatcattgatagagtataatatctttgttcattagagcgataaacttgaatttgagagggaacttccatggataaaaagtacagtattggtctagacataggaactaactctgttgggtgggctgttataacagatgaatataaagttccatcaaaaaaatttaaagtattaggaaacactgatagacattcaataaaaaaaaacttgataggtgctttattattcgattcaggagagactgctgaagctacacgtttaaaaagaacagctagacgtagatatacaagaagaaaaaataggatatgttatcttcaagaaatttttagtaatgaaatggcaaaagttgatgattcattctttcacagactagaagaaagtttcttagttgaagaagataagaagcatgaaagacaccctatttttggtaatatcgtagatgaagtagcatatcatgagaagtatccaactatctatcatttaagaaagaaattagttgattctacagataaagctgatctgagattaatatatttagctttagctcatatgattaaatttagaggacattttttaatagaaggtgatttaaacccagacaacagcgatgtagataaattatttatccaattagttcaaacttataatcaattattcgaagagaatccaattaatgcaagtggtgtagacgctaaggctatattatcagctagattatcaaaatctagaagattagaaaatctaatagctcaacttcctggagaaaagaaaaatggactttttgggaacctaatagctctctcactcggactaacaccaaattttaaaagcaattttgatcttgctgaagacgcaaagttacaactatcaaaggatacatacgatgatgatttagataatttgttagctcaaataggtgatcaatatgctgatttgtttcttgcagcaaaaaacttaagtgatgcaattttactatcagatatacttagagtaaatacagaaataacaaaggctcctttatcagcaagtatgattaaacgatatgatgagcatcatcaagatttaacattattaaaggcacttgtaagacaacaattaccagaaaaatataaagaaattttctttgatcaatctaaaaatggatatgctggatatatagacggtggagcaagtcaagaagagttttataaatttataaagcctattttagaaaaaatggatggaactgaagaattacttgttaaacttaacagagaagatttacttagaaaacaaagaacttttgataatggttcaattcctcaccaaattcatttaggagaattacatgctatactaagaagacaagaagatttttatccatttcttaaagataatagagaaaaaattgaaaaaattttaacttttagaataccatattatgtaggaccacttgcaaggggaaattcaagatttgcatggatgactagaaaatcagaagaaactataaccccgtggaattttgaagaagtagtagataaaggagctagtgctcaatcatttatagaaagaatgacaaattttgataagaatcttcctaacgaaaaggttttgccaaagcatagccttctttatgagtattttacagtttataatgagcttactaaagtaaaatacgttacagaaggaatgagaaaaccagcatttttgtctggtgaacaaaagaaagcaatagtagacctattatttaaaacaaataggaaggttaccgtaaagcaacttaaagaagattacttcaaaaaaattgaatgctttgatagtgttgaaatatcaggagttgaagatagatttaatgcttcacttggtacatatcacgatctcttaaaaattataaaagataaggattttttagataatgaagaaaatgaagatattcttgaagatatagtattaacattgacactttttgaagatagagaaatgatagaagaaagattaaaaacatatgcacatctttttgatgataaggttatgaagcaacttaaaagaagaagatatacaggttggggacgtttgtcaagaaagctaattaatggtattagagataaacaatcaggaaagactattctcgattttcttaaatcagatggatttgctaatagaaactttatgcaattaattcatgatgattctcttactttcaaagaggatattcaaaaggctcaagtttctggacaaggcgatagcttacacgaacacattgctaaccttgcagggagccccgctatcaaaaaaggaattttacaaacagttaaagttgtagatgaacttgttaaagttatgggaagacacaaacctgagaatatagttatagaaatggccagagaaaatcaaacaacacaaaaaggacaaaaaaattctagagagagaatgaagagaattgaagaaggaataaaagagctaggatcacaaatattaaaagaacatccagttgaaaatactcaattgcaaaatgaaaagttatatttgtattacttacaaaatggaagagatatgtatgttgatcaagaactcgatattaatagattaagtgactatgatgttgatcatattgttcctcaatcatttttaaaagatgattcaatcgataacaaagtattaactagatcagataaaaatagaggaaagtcagataatgtaccatctgaagaagttgttaaaaaaatgaagaactattggagacaacttttaaatgcaaagctaattacacaaagaaaatttgacaatttaacaaaagcagaaagaggaggattaagcgaattagacaaagctggatttataaaaagacaacttgttgagacaagacaaataactaagcatgttgctcaaatacttgattcaagaatgaatacaaaatatgatgaaaatgataaattaatcagagaagtaaaagtaataacattaaagtcaaaattagtatcagatttcagaaaggattttcaattttacaaagttcgtgaaataaataactatcatcatgctcatgatgcatacttaaatgctgttgtaggaactgctcttattaagaaatatcctaaactagaaagcgaatttgtttatggagattataaagtttatgatgtgcgcaaaatgatcgcgaaatccgaacaagaaatcggtaaggctacagcaaaatatttcttttatagtaatataatgaatttttttaagacagaaataactttggctaatggtgaaatcagaaaaagaccacttatcgaaacaaatggagagacaggagaaatagtatgggataaaggaagagattttgctactgttagaaaagtactaagtatgccacaagtaaatatcgtaaagaaaactgaagttcaaactggaggtttctctaaggaatcaattttacctaagagaaattcagataagttaattgcaaggaaaaaagattgggacccaaaaaaatacggtggttttgatagtccaacagttgcctatagtgttcttgtagtagcgaaagttgagaaaggtaagtcaaaaaagttgaaaagcgtaaaagaacttcttggtatcacaattatggaaagatcttcatttgaaaaaaatccaattgactttttagaagctaagggttataaagaagttaaaaaggatttaatcataaaactaccaaagtatagtctatttgaactcgaaaacggaagaaaacgaatgctcgctagcgcaggagaacttcaaaaaggaaatgaacttgcgctgccatcaaagtatgtaaatttcttatatttagcttctcattatgagaaattaaaaggatcaccagaggataatgaacaaaagcaactatttgtagaacaacacaaacattatttagatgaaataatagaacaaatatctgaattttctaaaagagttatacttgccgacgcaaatctagataaggtgctttcagcgtataataaacacagagataaaccaataagagaacaagcagaaaacattatccatctttttacattaactaatcttggtgcaccagctgcatttaagtactttgatacaacaatagatagaaaaagatacacatctactaaagaagtattagacgcaactttaatacatcaatctattacagggctttatgaaacaagaattgatttaagtcaactaggcggagattaagtcgacaaagtattgttaaaaataactctgtagaattataaattagttctacagagttattttttgacccgggtatattgataaaaataataatagtgggtataattaagttgttaggaggttagttagaatgatgtcaagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttgagctcttagttcaactcactttttaaggtgattgtttgcatgtcattataaaattcttcttcatcctcgtattcttgattccaaccgtttttaaatgcagatatgaatttttcaactattgattcattttcactttcagaaattacatactcgtttccatcattattaactctaataattagctgtgttatactattgctatccgtaccactcaatttcactgtgtaatctttgttttttatttctctaattaagtcattaatattcatttcagccctcctgtgaaattgttatccgctcacaattccacgtcgactaccgcggattctagattctgcagtatcttcatggtattcattttttaatatcattttaccctcccaatacatttaaaataattatgtattcatgaaacatgattgtatatttaagaaacataattccatataaatcatttttcaaaatagtttttacccataattaaatgttaatatgtaaattaatcttttagaatagttaaaaagttctaaaatatgttataatgtttcttataatcttataaattttaataactaatatataaagatatttctttaaaatattcttatatttagaagaatttattttaaaataaaaagcttttatgttgataaactgctttgcaaagctctcatgtaaatgtttaatataagactactataaaattggctaattttataggttaggaggtagaaatgcaaatattgtggaaaaagtatgttaaagaaaactttgaaatgaatgtagatgaatgtggtatagaacaaggtataccaggattaggatataactatgaagtattgaaaaatgctgttattcattacgtaactaagggatatggaacttttaaatttaatggtaaggtatataacttaaaacaaggtgatatttttatactactaaaaggtatgcaagttgagtatgtggcttctattgatgatccttgggaatactactggataggatttagtggttcaaatgctaatgagtatttaaatagaacttctattactaactcctgtgttgctaattgtgaagaaaactcaaaaattccacagataatattaaatatgtgcgaaatatcaaaaacttataatccttcaagatctgatgacatactattactaaaagaactttactcattattgtacgcacttatagaagaattcccaaaaccttttgaatacaaagataaggaattacacacatatattcaagatgctcttaatttcattaattctaattacatgcatagcataactgttcaagaaattgctgattatgtgaacttaagtagaagttatttatataaaatgttcataaaaaaccttggaatttctcctcaaagatatttaataaaccttagaatgtacaaagccacccttttattaaaaagcactaaacttcctataggagaagtcgcaagtagtgtaggttatagtgactccctgttattttcaaaaactttttcaaaacatttttcaatgtctccactaaattacagaaataatcaagtaaataaaccaagtatataaatttaaaatacagctttaaaacaaaaaaatttcaaaaataaaaagtataacagaggcgtaaattaaaacctctgttatactttttgagct

SEQ ID NO: 24 - DNA, 5754 bp, Artificial Sequence
  pEC750S-uppHR
ataaggtaccaggaattagagcagcgctatgttcagatacatttagtgctcatgcaacaagagaacataataatgctaatatattaactatgggtcaaagggttgttggagcaggtcttgctttagatatagtaaaaacatttatatcagctaaatttgaaggagataggcaccaaaaaagaatagataagatttcagatattgaaaaaaagtatacacattagaaaaaagcagctatgctgcaaataagatcaatttatattagaaaaaagcagctatgctgcaaataagatcaatttatattagaaaaaagcagctatgctgcaaataagatcaatttatattagaaaaaagcagctatgctacaaataagatcaatttatattagaaaaaagtagctatgctgcaacaatattaatttatattactagaaagctaaatggggtatataaatataaagggctataaatactaaaagcaaacttggaggaataataatggtctagagctggagatagattatttggtactaagtaattagtaatctattagaattaaaagctatctacataagtttctgaatgacccaagataattttactggggggaatatagaaaatggagagacgagataagaaaaattattacttggatattgctgaaacagttttagagagaggaacctgtctaaggagaaactatggttctataattgttaaaaatgatgaaataatttctactggatacacaggagcacctagaggtagaaaaaattgcatggatttgaatagttgcataagagaaaagttgaaagttccaagaggtactcattatgagttgtgtaggagtgtacatagtgaagctaatgcaataataagcgcttcgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccgccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagttgttaacttcaggtttgtctgtaactaaaaactagtatttaacctaggatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcaggcctcgagatctccatggacgcgtgacgtcgac

SEQ ID NO: 25 - DNA, 884 bp, Artificial Sequence
  Repair template
ataaggtaccaggaattagagcagcgctatgttcagatacatttagtgctcatgcaacaagagaacataataatgctaatatattaactatgggtcaaagggttgttggagcaggtcttgctttagatatagtaaaaacatttatatcagctaaatttgaaggagataggcaccaaaaaagaatagataagatttcagatattgaaaaaaagtatacacattagaaaaaagcagctatgctgcaaataagatcaatttatattagaaaaaagcagctatgctgcaaataagatcaatttatattagaaaaaagcagctatgctgcaaataagatcaatttatattagaaaaaagcagctatgctacaaataagatcaatttatattagaaaaaagtagctatgctgcaacaatattaatttatattactagaaagctaaatggggtatataaatataaagggctataaatactaaaagcaaacttggaggaataataatggtctagagctggagatagattatttggtactaagtaattagtaatctattagaattaaaagctatctacataagtttctgaatgacccaagataattttactggggggaatatagaaaatggagagacgagataagaaaaattattacttggatattgctgaaacagttttagagagaggaacctgtctaaggagaaactatggttctataattgttaaaaatgatgaaataatttctactggatacacaggagcacctagaggtagaaaaaattgcatggatttgaatagttgcataagagaaaagttgaaagttccaagaggtactcattatgagttgtgtaggagtgtacatagtgaagctaatgcaataataagcgcttc

SEQ ID NO: 26 - DNA, 500 bp, Artificial Sequence
  upp gene upstream fragment
ataaggtaccaggaattagagcagcgctatgttcagatacatttagtgctcatgcaacaagagaacataataatgctaatatattaactatgggtcaaagggttgttggagcaggtcttgctttagatatagtaaaaacatttatatcagctaaatttgaaggagataggcaccaaaaaagaatagataagatttcagatattgaaaaaaagtatacacattagaaaaaagcagctatgctgcaaataagatcaatttatattagaaaaaagcagctatgctgcaaataagatcaatttatattagaaaaaagcagctatgctgcaaataagatcaatttatattagaaaaaagcagctatgctacaaataagatcaatttatattagaaaaaagtagctatgctgcaacaatattaatttatattactagaaagctaaatggggtatataaatataaagggctataaatactaaaagcaaacttggaggaataataatg

SEQ ID NO: 27 - DNA, 377 bp, Artificial Sequence
  upp gene downstream fragment
gctggagatagattatttggtactaagtaattagtaatctattagaattaaaagctatctacataagtttctgaatgacccaagataattttactggggggaatatagaaaatggagagacgagataagaaaaattattacttggatattgctgaaacagttttagagagaggaacctgtctaaggagaaactatggttctataattgttaaaaatgatgaaataatttctactggatacacaggagcacctagaggtagaaaaaattgcatggatttgaatagttgcataagagaaaagttgaaagttccaagaggtactcattatgagttgtgtaggagtgtacatagtgaagctaatgcaataataagcgcttc

SEQ ID NO: 28 - DNA, 2666 bp, Artificial Sequence
  pEX-A2-gRNA-upp
ctcgagtatttttgataaaagcaatgattaacatggtttgacgtctgagaagagacgattttctcaataggagaaattaaggtgcaaacccttatcattccaccatgatccacctgtagcaagcatgttttagagctagaaatagcaagttaaaataaggctagtccgttatcaacttgaaaaagtggcaccgagtcggtgctttttttgccatggacctgcttttgctcgcttggatccgaattcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgactcccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaattgggtaccgagctcgcggccgcaagc

SEQ ID NO: 29 - DNA, 203 bp, Artificial Sequence
  gRNA expression cassette
tatttttgataaaagcaatgattaacatggtttgacgtctgagaagagacgattttctcaataggagaaattaaggtgcaaacccttatcattccaccatgatccacctgtagcaagcatgttttagagctagaaatagcaagttaaaataaggctagtccgttatcaacttgaaaaagtggcaccgagtcggtgcttttttt

SEQ ID NO: 30 - DNA, 100 bp, Artificial Sequence
  Constitutive promoter
tatttttgataaaagcaatgattaacatggtttgacgtctgagaagagacgattttctcaataggagaaattaaggtgcaaacccttatcattccaccat

SEQ ID NO: 31 - DNA, 20 bp, Artificial Sequence
  Protospacer targeting upp
gatccacctgtagcaagcat

SEQ ID NO: 32 - DNA, 5954 bp, Artificial Sequence
  pEC750S-deltaupp
ataaggtaccaggaattagagcagcgctatgttcagatacatttagtgctcatgcaacaagagaacataataatgctaatatattaactatgggtcaaagggttgttggagcaggtcttgctttagatatagtaaaaacatttatatcagctaaatttgaaggagataggcaccaaaaaagaatagataagatttcagatattgaaaaaaagtatacacattagaaaaaagcagctatgctgcaaataagatcaatttatattagaaaaaagcagctatgctgcaaataagatcaatttatattagaaaaaagcagctatgctgcaaataagatcaatttatattagaaaaaagcagctatgctacaaataagatcaatttatattagaaaaaagtagctatgctgcaacaatattaatttatattactagaaagctaaatggggtatataaatataaagggctataaatactaaaagcaaacttggaggaataataatggtctagagctggagatagattatttggtactaagtaattagtaatctattagaattaaaagctatctacataagtttctgaatgacccaagataattttactggggggaatatagaaaatggagagacgagataagaaaaattattacttggatattgctgaaacagttttagagagaggaacctgtctaaggagaaactatggttctataattgttaaaaatgatgaaataatttctactggatacacaggagcacctagaggtagaaaaaattgcatggatttgaatagttgcataagagaaaagttgaaagttccaagaggtactcattatgagttgtgtaggagtgtacatagtgaagctaatgcaataataagcgcttcgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccgccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagttgttaacttcaggtttgtctgtaactaaaaactagtatttaacctaggatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagtatttttgataaaagcaatgattaacatggtttgacgtctgagaagagacgattttctcaataggagaaattaaggtgcaaacccttatcattccaccatgatccacctgtagcaagcatgttttagagctagaaatagcaagttaaaataaggctagtccgttatcaacttgaaaaagtggcaccgagtcggtgctttttttgccatggacgcgtgacgtcgac

SEQ ID NO: 33 - DNA, 5853 bp, Artificial Sequence
  pEC750C-deltaupp
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagtatttttgataaaagcaatgattaacatggtttgacgtctgagaagagacgattttctcaataggagaaattaaggtgcaaacccttatcattccaccatgatccacctgtagcaagcatgttttagagctagaaatagcaagttaaaataaggctagtccgttatcaacttgaaaaagtggcaccgagtcggtgctttttttgccatggacgcgtgacgtcgacataaggtaccaggaattagagcagcgctatgttcagatacatttagtgctcatgcaacaagagaacataataatgctaatatattaactatgggtcaaagggttgttggagcaggtcttgctttagatatagtaaaaacatttatatcagctaaatttgaaggagataggcaccaaaaaagaatagataagatttcagatattgaaaaaaagtatacacattagaaaaaagcagctatgctgcaaataagatcaatttatattagaaaaaagcagctatgctgcaaataagatcaatttatattagaaaaaagcagctatgctgcaaataagatcaatttatattagaaaaaagcagctatgctacaaataagatcaatttatattagaaaaaagtagctatgctgcaacaatattaatttatattactagaaagctaaatggggtatataaatataaagggctataaatactaaaagcaaacttggaggaataataatggtctagagctggagatagattatttggtactaagtaattagtaatctattagaattaaaagctatctacataagtttctgaatgacccaagataattttactggggggaatatagaaaatggagagacgagataagaaaaattattacttggatattgctgaaacagttttagagagaggaacctgtctaaggagaaactatggttctataattgttaaaaatgatgaaataatttctactggatacacaggagcacctagaggtagaaaaaattgcatggatttgaatagttgcataagagaaaagttgaaagttccaagaggtactcattatgagttgtgtaggagtgtacatagtgaagctaatgcaataataagcgcttcgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 34 - DNA, 4966 bp, Artificial Sequence
  pGRNA-pNF2
agctcggtacccggggatcctctagagtcgacgtcacgcgtccatggagatctcgaggcgtgctagcttataatataatattgtggaatattgaaattaattttactaaaaactattccaatcaaggcagcaataataaaaatagttttatttattggtttagagtacgtgtttttatttttaagtttctttttaatagctgttagaaagaaacttaaaaataaaatatatataataataaatataataaaaatatatataaatttagaaaaagataatatttgataaaacatatttaaagatataatcaaatcctgtaaagatgaaaatgaaaataaaattcctaaaaataagtttaataaagccggaaatttcactagaaatttcactagtttttttaaaaacaataaatataataaagatattattataaaacttaaatttaataataacatcataaggaaaatggattggtttacttcaacggctttattatatgaaaaaaatacaattaaatttaaacataatattacaaaaagtattgttaaaattgaaaaaaattaaatctttagtatctttggaaataatcataatttataagctcctttgattttttatataaattatacattaattatttaatatataaaaactaaaattagtaaatcatctaaatattacaaatgaagaaatattatttgtaaataattatagtaatatattatggaatgaaatagtaaaaaattaaaaagaatcatttaatttagtttaaaatattttaataaaaataataaattaaaacataaaaaatagattaagtttcaattggggacttactctattttttattaagagtttaagaatagttaataattattgaaaatatgatataattttcttagttttacagaaggggaggtgaataagtgagaaacgcattaaagctgaataaaaagaactatatagataatattcatagtagaagtaaagggtggataaccaggagcgttatagataaaaaaggatatagccaatggcactataaatacgctgaattaaaagatttagatatgagtgatgaaaatatctatataactctaaataccttttataagccgtgtaggcgattagaaaatataaaagagttaaatacactgtttatagacttggattattataaaactggcaaaactaaagaccaggtattaatggacttagaaaagaattattttaatcaaagtattcctataccaaactatgtaatagatagtggaagaggaatgtatttaatatggataataaatgcagtacctagtaaagcattaccattatggaaagcggttcaagaatatttatataatcaattaaaatactttggagcagatagacaagccttagatgcaacgagaatattaagagttccaggaagtataaactctaaatcaaaaacagtagtcaatatattagatgagtacgaatatatttatgacttaagagaaatacaaaatggatttttacctgaattaaaaccatatgaaaggaaaaagggtagaccaagcaaaataaattatatttatagagaaagaagtttatattatggaagaatacaagacataataaaactttgtgaactaagagaatatgatttaaaaggacacagagagcttatattatttttatatagatattatctttgtagctttacagaagacattgagaaggcattaaatgatgttttagaacttaatagtatgtttagacaacatttaagtgaaagagaagttataagagcaactagaagtgctgaaagatgttatttagataaaaataagcaatataagtataagaatgaaactcttatagagttattagaaattacagaagaagaacaaaaatatatgacaataataatttctaaaaaggaatataagagaagagaaaatattagaggtaaaaaaaattatcaagagcaattaaaagctaaaggaaaagcaacaaaaaaagaggaattaaatgtattaagaaaaaaaataaaagcccttaaagaaaaaggctttaaaaataaagaaattactctaatgttagaagtaccaataaaaacattagaacgtcatattacgtatatgaaaaaaaatgggcttttataaaggctcattttttatattcttttcttcaaagattatataatataaaaaaatttttttcaaactttaaataaaaaatatttttatatttttttatttttttatttttatatttttttatttttttatttttatatttttttatttttatatttttttatttttatatttttttatttttttatttttttatttttttatttttttatttttttatttttttatttttaccctcattttttttacgcttgtattatagggtactttgtacctgttcttttttttggggaggttgtaaagataattttttactttagttagttccgaaggaacttttattatagtgcccctttatttttttgcgtggcattgaatgtaaaaaattatcactatactagggcgtaaagtaatattacatgtgtctcaaagtgggattaaagcgggattttatagggcgtgtttgtggcttagagtgggattattggaaatttttttgatcctaggttaaatactagtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtaggtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccggggatcgatccccggccgagcgcttagtgggaatttgtaccccttatcgatacaaattccccgtaggcgctagggacctctttagaccggtcagctgtcagacacttatcacattaagtatatactagtattaagctagctttggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcactctatcattgatagagtttgaaactctatcattgatagagtataatatctttgttcatttaagccatctactaaacaagttttagagctagaaatagcaagttaaaataaggctagtccgttatcaacttgaaaaagtggcaccgagtcggtgctttttttgaagcttg

SEQ ID NO: 35 - DNA, 400 bp, Artificial Sequence
  catB gene upstream fragment
gtctttacacttttgcccattaatttttgagttccttatttttagggagcttttattatttttatcatgaaaatttcataaaatactcataaactaaggatgtcttcataatcagattagtactccattttcaatccatttaatctgggaatatgatattttaattacgtattatttaagatatattaacgtgtaatataataccccgcaaatattaattatcacatacatatcccccctttattggggcattttttgtacccattattttagtattgtgcagtacttaaataaaaaaatgccgcaaattcatttttattgaataatgcggtatttcttctattctttatttttattactctataaataatgtaatcaagacatgactatctaaatatat

SEQ ID NO: 36 - DNA, 400 bp, Artificial Sequence
  catB gene downstream fragment
aattcataattcgggcctcctaaaaattttcgtaattctattttagaaggcttttttccgtgacctagccatttcaatctcctttttacaatgatatttacgctttagtttattatagcacattctgtaataccgaactattcaattttcagagaccattttttattgattcataacttaagaatactacgaattactctaatattttactttttcttatctcttgttattttaacatcggaattactactaatattaatttttatttttccatccgcatttgctccaacatttttttaactatactttccttttgttaataaattatgttattgttgaacaatataagaaaagtgcgtaacattttttattaaaaataattaggtatttctatctgtgg

SEQ ID NO: 37 - protein, 218 aa, Clostridium beijerinckii
MNFNLIDINHWSRKPYFEHYLNNVKCTYSMTANIEITDLLYEIKLKNIKFYPTLIYMIATVVNKHKEFRICDHEGSLGYWDSMNPSYTIFHKENETFSSIWTEYNKSFLRFYSDYLDDIKNYGNIMKFTPKSNEPDNTFSVSSIPWVSFTGFNLNVYNEGTYLIPIFTAGKYFKQENKIFIPISIQVHHAICDGYHASRFINEMQELAFSFQEWLENK

SEQ ID NO: 38 - DNA, 9113 bp, Artificial Sequence
  pCas9ind-gRNA_catB
catggataaaaagtacagtattggtctagacataggaactaactctgttgggtgggctgttataacagatgaatataaagttccatcaaaaaaatttaaagtattaggaaacactgatagacattcaataaaaaaaaacttgataggtgctttattattcgattcaggagagactgctgaagctacacgtttaaaaagaacagctagacgtagatatacaagaagaaaaaataggatatgttatcttcaagaaatttttagtaatgaaatggcaaaagttgatgattcattctttcacagactagaagaaagtttcttagttgaagaagataagaagcatgaaagacaccctatttttggtaatatcgtagatgaagtagcatatcatgagaagtatccaactatctatcatttaagaaagaaattagttgattctacagataaagctgatctgagattaatatatttagctttagctcatatgattaaatttagaggacattttttaatagaaggtgatttaaacccagacaacagcgatgtagataaattatttatccaattagttcaaacttataatcaattattcgaagagaatccaattaatgcaagtggtgtagacgctaaggctatattatcagctagattatcaaaatctagaagattagaaaatctaatagctcaacttcctggagaaaagaaaaatggactttttgggaacctaatagctctctcactcggactaacaccaaattttaaaagcaattttgatcttgctgaagacgcaaagttacaactatcaaaggatacatacgatgatgatttagataatttgttagctcaaataggtgatcaatatgctgatttgtttcttgcagcaaaaaacttaagtgatgcaattttactatcagatatacttagagtaaatacagaaataacaaaggctcctttatcagcaagtatgattaaacgatatgatgagcatcatcaagatttaacattattaaaggcacttgtaagacaacaattaccagaaaaatataaagaaattttctttgatcaatctaaaaatggatatgctggatatatagacggtggagcaagtcaagaagagttttataaatttataaagcctattttagaaaaaatggatggaactgaagaattacttgttaaacttaacagagaagatttacttagaaaacaaagaacttttgataatggttcaattcctcaccaaattcatttaggagaattacatgctatactaagaagacaagaagatttttatccatttcttaaagataatagagaaaaaattgaaaaaattttaacttttagaataccatattatgtaggaccacttgcaaggggaaattcaagatttgcatggatgactagaaaatcagaagaaactataaccccgtggaattttgaagaagtagtagataaaggagctagtgctcaatcatttatagaaagaatgacaaattttgataagaatcttcctaacgaaaaggttttgccaaagcatagccttctttatgagtattttacagtttataatgagcttactaaagtaaaatacgttacagaaggaatgagaaaaccagcatttttgtctggtgaacaaaagaaagcaatagtagacctattatttaaaacaaataggaaggttaccgtaaagcaacttaaagaagattacttcaaaaaaattgaatgctttgatagtgttgaaatatcaggagttgaagatagatttaatgcttcacttggtacatatcacgatctcttaaaaattataaaagataaggattttttagataatgaagaaaatgaagatattcttgaagatatagtattaacattgacactttttgaagatagagaaatgatagaagaaagattaaaaacatatgcacatctttttgatgataaggttatgaagcaacttaaaagaagaagatatacaggttggggacgtttgtcaagaaagctaattaatggtattagagataaacaatcaggaaagactattctcgattttcttaaatcagatggatttgctaatagaaactttatgcaattaattcatgatgattctcttactttcaaagaggatattcaaaaggctcaagtttctggacaaggcgatagcttacacgaacacattgctaaccttgcagggagccccgctatcaaaaaaggaattttacaaacagttaaagttgtagatgaacttgttaaagttatgggaagacacaaacctgagaatatagttatagaaatggccagagaaaatcaaacaacacaaaaaggacaaaaaaattctagagagagaatgaagagaattgaagaaggaataaaagagctaggatcacaaatattaaaagaacatccagttgaaaatactcaattgcaaaatgaaaagttatatttgtattacttacaaaatggaagagatatgtatgttgatcaagaactcgatattaatagattaagtgactatgatgttgatcatattgttcctcaatcatttttaaaagatgattcaatcgataacaaagtattaactagatcagataaaaatagaggaaagtcagataatgtaccatctgaagaagttgttaaaaaaatgaagaactattggagacaacttttaaatgcaaagctaattacacaaagaaaatttgacaatttaacaaaagcagaaagaggaggattaagcgaattagacaaagctggatttataaaaagacaacttgttgagacaagacaaataactaagcatgttgctcaaatacttgattcaagaatgaatacaaaatatgatgaaaatgataaattaatcagagaagtaaaagtaataacattaaagtcaaaattagtatcagatttcagaaaggattttcaattttacaaagttcgtgaaataaataactatcatcatgctcatgatgcatacttaaatgctgttgtaggaactgctcttattaagaaatatcctaaactagaaagcgaatttgtttatggagattataaagtttatgatgtgcgcaaaatgatcgcgaaatccgaacaagaaatcggtaaggctacagcaaaatatttcttttatagtaatataatgaatttttttaagacagaaataactttggctaatggtgaaatcagaaaaagaccacttatcgaaacaaatggagagacaggagaaatagtatgggataaaggaagagattttgctactgttagaaaagtactaagtatgccacaagtaaatatcgtaaagaaaactgaagttcaaactggaggtttctctaaggaatcaattttacctaagagaaattcagataagttaattgcaaggaaaaaagattgggacccaaaaaaatacggtggttttgatagtccaacagttgcctatagtgttcttgtagtagcgaaagttgagaaaggtaagtcaaaaaagttgaaaagcgtaaaagaacttcttggtatcacaattatggaaagatcttcatttgaaaaaaatccaattgactttttagaagctaagggttataaagaagttaaaaaggatttaatcataaaactaccaaagtatagtctatttgaactcgaaaacggaagaaaacgaatgctcgctagcgcaggagaacttcaaaaaggaaatgaacttgcgctgccatcaaagtatgtaaatttcttatatttagcttctcattatgagaaattaaaaggatcaccagaggataatgaacaaaagcaactatttgtagaacaacacaaacattatttagatgaaataatagaacaaatatctgaattttctaaaagagttatacttgccgacgcaaatctagataaggtgctttcagcgtataataaacacagagataaaccaataagagaacaagcagaaaacattatccatctttttacattaactaatcttggtgcaccagctgcatttaagtactttgatacaacaatagatagaaaaagatacacatctactaaagaagtattagacgcaactttaatacatcaatctattacagggctttatgaaacaagaattgatttaagtcaactaggcggagattaagtcgacaaagtattgttaaaaataactctgtagaattataaattagttctacagagttattttttgacccgggtatattgataaaaataataatagtgggtataattaagttgttaggaggttagttagaatgatgtcaagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggctcgaggacgtcaaaccatgttaatcattgcttttatcaaaaataggatccactctatcattgatagagtttgaaactctatcattgatagagtataatatctttgttcatgtacatcatgctatctgtgagttttagagctagaaatagcaagttaaaataaggctagtccgttatcaacttgaaaaagtggcaccgagtcggtgctttttttgaagcttgtctttacacttttgcccctcgagtccctatcagtgatagattgaaactctatcattgatagagtataatatctttgttcattagagcgataaacttgaatttgagagggaacttc

SEQ ID NO: 39 - DNA, 20 bp, Artificial Sequence
  Primer pNF2
gggcgcacttatacaccacc

SEQ ID NO: 40 - DNA, 20 bp, Artificial Sequence
  Primer pNF2
tgctacgcaccccctaaagg

SEQ ID NO: 41 - DNA, 50 bp, Artificial Sequence
  DeltacatB_gRNA_rev
aatctatcactgatagggactcgaggggcaaaagtgtaaagacaagcttc

SEQ ID NO: 42 - DNA, 20 bp, Artificial Sequence
  Primer pCas9ind_fwd
agctcttgatccggcaaaca

SEQ ID NO: 43 - DNA, 20 bp, Artificial Sequence
  Primer pCas9ind _rev
gcaaccctagtgttcggtga

SEQ ID NO: 44 - protein, 219 aa, Clostridium butyricum
MNFNLIDINHWSRKPYFEHYLNNVKCTYSMTANIEITDLLYEIKLKNIKFYPTLIYMIATVVNNHKEFRICFDHKGSLGYWDSMNPSYTIFHKENETFSSIWTEYNKSFLRFYSDYLDDIKNYGNIMKFTPKSNEPDNTFSVSSIPWVSFTGFNLNVYNEGTYLIPIFTAGKYFKQENKIFIPISIQVHHAICDGYHASRFINEMQELAFSFQEWLENK

SEQ ID NO: 45 - protein, 219 aa, Clostridium beijerinckii
MNFNLIDINHWIRKPYFEHYLNNVKCTYSMTANIEITDLLYEIKLKNIKFYPTLIYMIATVVNNHKEFRICFDHKGSLGYWDSMNPSYTIFHKENETFSSIWTEYNKSFLRFYSDYLDDIKNYGNIMKFTPKSNEPDNTFSVSSIPWVSFTGFNLNVYNEGTYLIPIFTAGKYFKQENKIFIPISIQVHHAICDGYHASRFINEMQELAFSFQEWLENK

SEQ ID NO: 46 - protein, 219 aa, Clostridium beijerinckii
MNFNLIDINHWSRKPYFEHYLNNVKCTYSMTANIEITDLLYEIKLKNIKFYPTLIYMIATVVNNHEEFRICFDHEGSLGYWDSMNPSYTIFHKENETFSSIWTEYNKSFLRFYSDYLDDIKNYGNIMKFTPKSNEPDNTFSVSSIPWVSFTGFNLNVYNEGTYLIPIFTAGKYFKQGNKVFIPISIQVHHAICDGYHASRFINEMQELAFSFQEWLENK

SEQ ID NO: 47 - protein, 219 aa, Clostridium beijerinckii
MNFNLIDINHWSRKPYFEHYLNNVKCTYSMTANIEITDLLYEIKLKNIKFYPTLIYMIATVVNNHKEFSICFDHEGSLGYWDSMNPSYTIFHKENETFSSIWTEYNESFLRFYSDYLDDIKNYGNIMKFTPKSNEPDNTFSVSSIPWVSFTGFNLNVYNEGTYLIPIFTAGKYFKQGNKIFIPISIQVHHAICDGYHASRFINEMQELAFSFQEWLENK

SEQ ID NO: 48 - protein, 219 aa, Artificial Sequence
  Clostridium sp.2-1
MNFNLIDINHWSRKPYFEHYLNNVKCTYSMTANIEITDLLYEIKLKNIKFYPTLIYMIANVVNNHKEFRICFDHNGSLGYWDSMNPSYTIFHKENETFSSIWTEYNESFLRFYSDYLDDIKNYGNIMKFTPKSNQPDNTFSVSSIPWVSFTGFNLNVYNEGTYLIPIFTAGKYFKQENKIFIPISIQVHHAICDGYHASRFINEMQELAFSFQEWLENK

SEQ ID NO: 49 - protein, 219 aa, Artificial Sequence
  Clostridium diolis
MNFNLIDINHWSRKPYFEHYLNNVKCTYSMTANIEITDLLYEIKLKNIKFYPTLIYMIANVVNNHKEFRICFDHNGSLGYWDSMNPSYTVFHKENETFSSIWTEYNESFLRFYSDYLDDIKNYGNIMKFTPKSNEPDNTFSVSSIPWVSFTGFNLNVYNEGTYLIPIFTAGKYFKQGNKIFIPISIQVHHAICDGYHASRFINEMQELAFSFQEWLENK

SEQ ID NO: 50 - protein, 219 aa, Clostridium beijerinckii
MNFNLIDINHWIRKPYFEHYLNNVKCTYSMTANIEITDLLYEIKLKNIKFYPTLIYMIATVVNNHKEFRICFDHKGSLGYWDSMNPSYTIFHKENETFSSIWTEYNKSFLRFYSDYLDDIKNYGNIMKFTPKSNQPDNTFSVSSIPWVSFTGFNLNVNNEGTYLIPIFTAGKYFKQENKIFIPISIQVHHAICDGYHASRFINEMQELAFSFKEWLENK

SEQ ID NO: 51 - protein, 221 aa, Clostridium beijerinckii
MNFNLIDINNWSRKPYFEHYLNNVKCTYSMTANIEITDLLYEIKLKNIKFYPTLIYMIANVVNNHKEFRICFDHEGSLGYWDSMNPSYTIFHKENETFSSIWTEYNESFLRFYSDYLDDIKNYGNIMKFTPKSNEPDNTFPVSSIPWVSFTGFNLNVYNEGTYLIPIFTAGKYFKQGNKIFIPISIQVHHAICDGYHASRFINEMQELAFSFQEWLENKYI

SEQ ID NO: 52 - protein, 219 aa, Clostridium beijerinckii
MNFNLIDINNWSRKPYFEHYLNNVKCTYSMTANIEITDLLYEIKLKNIKFYPTLIYMIATVVNNHKEFRICFDHNGSLGYWDSMNPSYTIFHKENETFSSIWTEYNESFLRFYSDYLDDIKNYGNIMKFTPKSNEPDNTFPVSSIPWVSFTGFNLNVYNEGTYLIPIFTAGKYFKQGNKIFIPISIQVHHAICDGYHASRFINEMQELAFSFREWLENK

SEQ ID NO: 53 - protein, 219 aa, Clostridium saccharoperbutylacetonicum
MNFNLIDINHWSRKPYFEHYLNTVKCTYSMTANIEITDLLYEIKLKNIKFYPTLIYMIATVVNNHKEFRICFDHKGSLGYWDSMNPSYTIFHKENETFSSIWTEYNESFLRFYSDYIDDIKNYGNIMKFTPKSNEPDNIFPVSSIPWVSFTGFNLNVYNEGTYLIPIFTAGKYFKQENKIFIPISIQVHHAVCDGYHASRFINEMQELAFSFQEWLENK

SEQ ID NO: 54 - protein, 219 aa, Clostridium saccharoperbutylacetonicum
MNFNLIDINHWSRKPYFEHYLNTVKCTYSMTANIEITDLLYEIKLKNIKFYPTLIYMIATVVNNHKEFRICFDHKGSLGYWDSMNPSYTIFYKENETFSSIWTEYNESFLRFYSDYIDDIKNYGNIMKFTPKSNEPDNIFPVSSIPWVSFTGFNLNVYNEGTYLIPIFTAGKYFKQENKIFIPISIQVHHAVCDGYHASRFINEMQELAFSFQEWLENK

SEQ ID NO: 55 - protein, 219 aa, Clostridium beijerinckii
MNFNLIDINNWSRKPYFEHYLNNVKCTYSMTANIEITDLLYEIKLKNIKFYPTLIYMIATVVNNHKEFRICFDHNGSLGYWDSMNPSYTIFHKENETFSSIWTEYNESFLRFYSDYLDDIKNYGNIMKFTPKSNRSDNTFPVSSIPWVSFTGFNLNVYNEGTYLIPIFTAGKYFKQGNKIFIPISIQVHHAICDGYHASRFINEMQELAFSFREWLENK

SEQ ID NO: 56 - protein, 221 aa, Clostridium beijerinckii
MNFNLIDINHWNRKPFFEHYLNNVKCTYSMTANIEITDLLYEIKLKNIKFYPTLIYMIATVVNNHKEFRICFDHKGSLGYWDSMNPSYTIFHEENETFSSIWTEYNESFLRFYSDYLDDIKNYGNIMKFTPKSNEPDNTFPVSSIPWVSFTGFNLNVCNEGTYLTPIFTAGKYFKQENKIFIPISIQVHHSICDGYHASRFINEMQELAFSFQEWLENKYI

SEQ ID NO: 57 - protein, 219 aa, Clostridium beijerinckii
MNFNLIDIKHWSRKPYFEHYLNNVKCTYSMTANIEITDLLYEIRLKNIKLYPTLIYMIATVVNNHKEFRTCFDHSGSLGYWDSMSPSYTIFHKENETFSSIWTEYNESFPRFYSDYFDDIKNYGNIMKFTPKLNEPDNTFPVSSIPWVSFTGFNLNVYNEGTYLIPIFTTGKYFKQENKMFIPISIQVHHAICDGYHASRFINEMQELAFSFQDWLENK

SEQ ID NO: 58 - protein, 219 aa, Clostridium botulinum
MKFNLIDIEHWNRKPYFEYYLHSVRCTYSMTANIEITNLLHEIKLKKLKLYPTLIYIIATVVNNHKEFRTCFDENGNLGYWDSMSPSYTIFHKDNETFSSIWTDYDESFSCFYNDYLDDIKNYGAIMKFTPKLNEPANTFPVSSIPWVNFTGFNLNVYNNGTYLVPIFTMGKYFEQNNKIFIPMSIQVHHAVCDGYHISRFINEVQELALNSQTWLKHK

SEQ ID NO: 59 - protein, 219 aa, Artificial Sequence
  Anaerocolumna aminovalerica
MKFNLIDIENWNRKPYFEHYLNSVRCTYSMTANIEITNLLHEIKLKDLKLYPTLIYILATVVNNHKEFRTCFDENGNLGYWDSMSPSYTIFHKENETFSSIWTEYDESFSRFYTAYLDDIKNHGNIMKFTPKLNEPANTFPISSIPWVSFTGFNLNVYNDGKYLLPIFTTGKYFEQNSKIFIPMSVQVHHAVCDGYHISRFINEVQEVILNYQTWLGDK

SEQ ID NO: 60 - protein, 219 aa, Artificial Sequence
  Desnuesiella massiliensis
MKFNLIDIEHWNRKPYFEHYLNSVRCTYSMTANIEITNLLHDIKLKKLKLYPTLIYIIATVVNNHEEFRTCFYENGNLGYWDSMSPSYTIFHKDNETFSEIWSEYDESFSCFYSKYLDDIKNYGDIMRFTPKLNEPANTFPISCIPWVSFTGFNLNVYNDGRYLVPIFTIGKYFEQNNKIFIPMSIQVHHAVCDGYHTSRFINEVQELALNSQTWLRHK

SEQ ID NO: 61 - protein, 219 aa, Artificial Sequence
  Clostridium sp. HMP27
MKFNLIDTEHWNRKPYFEHYLNSVRCTYSITANIEITNLLHDIKQKKLKLYPTFIYIIATVVNTHKEFRTCFDESGNLGYWDSMSPSYTIFHKDNETFSSIWTEYDKSFSCFYSKYLHDIKNYGDIMSFTPKLNEPANTFPISCIPWVSFTGFNLNIYNDGTYLVPIFTIGKYFKQADKILIPISIQVHHAVCDGYHTSRFINEVQELILNYQTWLKHK

SEQ ID NO: 62 - protein, 219 aa, Artificial Sequence
  Clostridium drakei
MKFNLIDIENWNRKPYFEHYLNAVRCTYSMTANIEITGLLREIKLKGLKLYPTLIYIITAVINRHKEFRTCFDENRKLGYWDSMSPSYTVFHKEDETFSSIWTEYDESFPRFYDNYLDDIKSYGDVLKFMPKPDEPGNTFNVSSIPWVNFTGFNLNIYNDATYLIPIFTMGKFFHQDNKIFIPMSIQVHHAVCDGYHTSRFFNEVQELSSNFETWLDEK

SEQ ID NO: 63 - protein, 219 aa, Clostridium scatologenes
MKFNLIDIEDWNRKPYFEHYLNAVRCTYSMTANIEITGLLREIKLKGLKLYPTLIYIITAVINRHKEFRTCFDENRKLGYWDSMSPSYTVFHKEDETFSSIWTEYDESFPRFYDNYLDDIKSYGDVLKFMPKPDEPGNTFNVSSIPWVNFTGFNLNIYNDATYLIPIFTMGKFFHQDNKIFIPMSIQVHHAVCDGYHTSRFFNEVQELSSNFETWLGEK

SEQ ID NO: 64 - protein, 219 aa, Artificial Sequence
  Clostridium tunisiense
MKFNLIDTEHWDRKPYFEHYFNSVKCTYSMTANIEITNLLNHIRLKKLKLYPTLIYIIATVVNNHEEFRICFDENNNLGYWDSMSPNYTIFHEDNKTFSSIWTEYEESFSGFYNKYLEDIKTYGHIMSFEPKLNESTNTFPISCIPWVSFTGFNLNIQDDGTYLTPIFTLGKYFEQNNKTFIPISIQVHHAVCDGYHTSRFINEVQELASDFQIWLTYK

SEQ ID NO: 65 - protein, 219 aa, Artificial Sequence
  Lachnospiraceae
MKFNLIDIEDWNRKPYFEHYLNAVRCTYSMTANIEITGLLREIKLKGLKLYPTLIYIITTVVNRHKEFRTCFDQKGKLGYWDSMNPSYTVFHKDNETFSSIWTEYDENFPRFYYNYLEDIRNYSDVLNFMPKTGEPANTINVSSIPWVNFTGFNLNIYNDATYLIPIFTLGKYFQQDNKILLPMSVQVHHAVCDGYHTSRFFNEAQELASNYETWLGEK

SEQ ID NO: 66 - protein, 219 aa, Clostridium perfringens
MKFNLIDIEDWNRKPYFEHYLNAVRCTYSMTANIEITGLLREIKLKGLKLYPTLIYIITTVVNRHKEFRTCFDQKGKLGYWDSMNPSYTVFHKDNETFSSIWTEYDENFPRFYYNYLEDIRNYSDVLNFMPKTGEPANTINVSSIPWVNFTGFNLNIYNDATYLIPIFTLGKYFQQDNKILLPMSVQVHHAVCDGYHISRFFNEAQELASNYETWLGEK

SEQ ID NO: 67 - protein, 218 aa, Artificial Sequence
  Clostrdium sp. BL8
MKFNLIDIDQWDRKPYFEHYFNSVKCTYSITANIEITNLLKDIKITKLKLYPTLIYIIATVINNHEEFRTCFDENNNLGYWDSMSPNYTIFHEETKTFSNIWTEYDKSFSGFYNKYVEDNKNYGNIMNFDPKLNEPANTFPISCIPWVSFTGFNLNIQDHGTYLTPIFTLGKYFEENNKVFIPMSIQVHHAVCDGYHTSRFINEVQELASNSQSWLKH

SEQ ID NO: 68 - DNA, 660 bp, Clostridium perfringens
atgaaatttaatttgatagatattgaggattggaatagaaagccatactttgagcattatttaaatgcggttaggtgcacttacagtatgactgcaaatatagagataactggtttactgcgtgaaattaaacttaagggcctgaaactgtaccctacgcttatttatatcatcacaactgtggttaaccgtcacaaggagttccgcacctgttttgatcaaaaaggtaagttaggatactgggatagtatgaacccaagttatactgtctttcataaggataacgaaactttttcaagtatttggacagagtatgacgagaacttcccacgtttttactataattaccttgaggatattagaaactatagcgacgttttgaatttcatgcctaagacaggtgaacctgctaatacaattaatgtgtccagcattccttgggtgaattttaccggattcaacctgaatatatacaatgatgcaacatatctaatccctatttttactttgggtaagtattttcagcaggataataaaattttattacctatgtctgtacaggtgcatcatgcggtttgcgacggttatcatataagcagattttttaatgaggcacaggaattagcgtcaaattatgagacatggttaggagaaaaataa

SEQ ID NO: 69 - DNA, 624 bp, Clostridium difficile
atggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaa

SEQ ID NO: 70 - DNA, 624 bp, Clostridium perfringens; identical to SEQ ID NO: 69

SEQ ID NO: 71 - DNA, 3897 bp, Artificial Sequence
  Optimized MAD7
ctcgagtccctatcagtgatagattgaaactctatcattgatagagtataatatctttgttcattagagcgataaacttgaatttgagagggaacttagatgaacaacggcacaaataattttcagaacttcatagggatatcaagtttgcagaaaacgttaagaaatgctttaatacccacggaaaccacgcaacagttcatagttaagaacggaataattaaagaagatgagttaagaggcgagaacagacagattttaaaagatataatggatgactactacagaggattcatatctgagactttaagttctattgatgacatagattggactagcttattcgaaaaaatggaaattcagttaaaaaatggtgataataaagataccttaattaaggaacagacagagtatagaaaagcaatacataaaaaatttgcgaacgacgatagatttaagaacatgtttagcgccaaattaattagtgacatattacctgaatttgttatacacaacaataattattcggcatcagagaaagaggaaaaaacccaggtgataaaattgttttcgagatttgcgactagctttaaagattacttcaagaacagagcaaattgcttttcagcggacgatatttcatcaagcagctgccatagaatagttaacgacaatgcagagatattcttttcaaatgcgttagtttacagaagaatagtaaaatcgttaagcaatgacgatataaacaaaatttcgggcgatatgaaagattcattaaaagaaatgagtttagaagaaatatattcttacgagaagtatggggaatttattacccaggaaggcattagcttctataatgatatatgtgggaaagtgaattcttttatgaacttatattgtcagaaaaataaagaaaacaaaaatttatacaaacttcagaaacttcacaaacagattctatgcattgcggacactagctatgaggttccgtataaatttgaaagtgacgaggaagtgtaccaatcagttaacggcttccttgataacattagcagcaaacatatagttgaaagattaagaaaaataggcgataactataacggctacaacttagataaaatttatatagtgtccaaattttacgagagcgttagccaaaaaacctacagagactgggaaacaattaataccgccttagaaattcattacaataatatattgccgggtaacggtaaaagtaaagccgacaaagtaaaaaaagcggttaagaatgatttacagaaatccataaccgaaataaatgaactagtgtcaaactataagttatgcagtgacgacaacataaaagcggagacttatatacatgagattagccatatattgaataactttgaagcacaggaattgaaatacaatccggaaattcacctagttgaatccgagttaaaagcgagtgagcttaaaaacgtgttagacgtgataatgaatgcgtttcattggtgttcggtttttatgactgaggaacttgttgataaagacaacaatttttatgcggaattagaggagatttacgatgaaatttatccagtaattagtttatacaacttagttagaaactacgttacccagaaaccgtacagcacgaaaaagattaaattgaactttggaataccgacgttagcagacggttggtcaaagtccaaagagtattctaataacgctataatattaatgagagacaatttatattatttaggcatatttaatgcgaagaataaaccggacaagaagattatagagggtaatacgtcagaaaataagggtgactacaaaaagatgatttataatttgttaccgggtcccaacaaaatgataccgaaagttttcttgagcagcaagacgggggtggaaacgtataaaccgagcgcctatatactagaggggtataaacagaataaacatataaagtcttcaaaagactttgatataactttctgtcatgatttaatagactacttcaaaaactgtattgcaattcatcccgagtggaaaaacttcggttttgattttagcgacaccagtacttatgaagacatttccgggttttatagagaggtagagttacaaggttacaagattgattggacatacattagcgaaaaagacattgatttattacaggaaaaaggtcaattatatttattccagatatataacaaagatttttcgaaaaaatcaaccgggaatgacaaccttcacaccatgtacttaaaaaatcttttctcagaagaaaatcttaaggatatagttttaaaacttaacggcgaagcggaaatattcttcaggaagagcagcataaagaacccaataattcataaaaaaggctcgattttagttaacagaacctacgaagcagaagaaaaagaccagtttggcaacattcaaattgtgagaaaaaatattccggaaaacatttatcaggagttatacaaatacttcaacgataaaagcgacaaagagttatctgatgaagcagccaaattaaagaatgtagtgggacaccacgaggcagcgacgaatatagttaaggactatagatacacgtatgataaatacttccttcatatgcctattacgataaatttcaaagccaataaaacgggttttattaatgataggatattacagtatatagctaaagaaaaagacttacatgtgataggcattgatagaggcgagagaaacttaatatacgtgtccgtgattgatacttgtggtaatatagttgaacagaaaagctttaacattgtaaacggctacgactatcagataaaattaaaacaacaggagggcgctagacagattgcgagaaaagaatggaaagaaattggtaaaattaaagagataaaagagggctacttaagcttagtaatacacgagatatctaaaatggtaataaaatacaatgcaattatagcgatggaggatttgtcttatggttttaaaaaagggagatttaaggttgaaagacaagtttaccagaaatttgaaaccatgttaataaataaattaaactatttagtatttaaagatatttcgattaccgagaatggcggtttattaaaaggttatcagttaacatacattcctgataaacttaaaaacgtgggtcatcagtgcggctgcattttttatgtgcctgctgcatacacgagcaaaattgatccgaccaccggctttgtgaatatatttaaatttaaagacttaacagtggacgcaaaaagagaattcattaaaaaatttgactcaattagatatgacagtgaaaaaaatttattctgctttacatttgactacaataactttattacgcaaaacacggttatgagcaaatcatcgtggagtgtgtatacatacggcgtgagaataaaaagaagatttgtgaacggcagattctcaaacgaaagtgataccattgacataaccaaagatatggagaaaacgttggaaatgacggacattaactggagagatggccacgatcttagacaagacattatagattatgaaattgttcagcacatattcgaaattttcagattaacagtgcaaatgagaaactccttgtctgaattagaggacagagattacgatagattaatttcacctgtattaaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgagagataaattaaaaataagcaataaagattggttcgactttatacagaataagagatatttataagtcgac

SEQ ID NO: 72 - protein, 1263 aa, Artificial Sequence
  MAD7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 73 - DNA, 363 bp, Artificial Sequence
  CatB promoter
taaaaaatgttacgcacttttcttatattgttcaacaataacataatttattaacaaaaggaaagtatagttaaaaaaatgttggagcaaatgcggatggaaaaataaaaattaatattagtagtaattccgatgttaaaataacaagagataagaaaaagtaaaatattagagtaattcgtagtattcttaagttatgaatcaataaaaaatggtctctgaaaattgaatagttcggtattacagaatgtgctataataaactaaagcgtaaatatcattgtaaaaaggagattgaaatggctaggtcacggaaaaaagccttctaaaatagaattacgaaaatttttaggaggcccgaatt

SEQ ID NO: 74 - DNA, 322 bp, Artificial Sequence
  CATQ promoter
ctgcgtacacatccagacatcgctttagagtatggtgaattaaagatggagcgggcttatcgattctcagaggatattgaaggctactgcactggtaaggatgcatttgtaaagcaactagaaaaggatgctttgcgatggtggcaaactgtctgttaggaggttattctcaaaggattgcaagaagcagttgaggataatccgtataactaactattacacattcttaacattgctggtttgtatcggtagaataacacgaattaacaaaggatatattttgtagtagcaagtgtatttgttttatattctatgaacctat

SEQ ID NO: 75 - protein, 1368 aa, Streptococcus pyogenes [sequence omitted]

SEQ ID NO: 76 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 77 - DNA, 1170 bp, Artificial Sequence
  bdhA
atgctaagttttgattattcaataccaactaaagttttttttggaaaaggaaaaatagacgtaattggagaagaaattaagaaatatggctcaagagtgcttatagtttatggcggaggaagtataaaaaggaacggtatatatgatagagcaacagctatattaaaagaaaacaatatagctttctatgaactttcaggagtagagccaaatcctaggataacaacagtaaaaaaaggcatagaaatatgtagagaaaataatgtggatttagtattagcaatagggggaggaagtgcaatagactgttctaaggtaattgcagctggagtttattatgatggcgatacatgggacatggttaaagatccatctaaaataactaaagttcttccaattgcaagtatacttactctttcagcaacagggtctgaaatggatcaaattgcagtaatttcaaatatggagactaatgaaaagcttggagtaggacatgatgatatgagacctaaattttcagtgttagatcctacatatacttttacagtacctaaaaatcaaacagcagcgggaacagctgacattatgagtcacacctttgaatcttactttagtggtgttgaaggtgcttatgtgcaggacggtatagcagaagcaatcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttattaa

SEQ ID NO: 78 - DNA, 1173 bp, Artificial Sequence
  bdhB
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 79 - DNA, 6560 bp, Artificial Sequence
  pGRNA-deltabdhB
gatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctaggatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagactctatcattgatagagtttgaaactctatcattgatagagtataatatctttgttcatgcttattacgacataacacagttttagagctagaaatagcaagttaaaataaggctagtccgttatcaacttgaaaaagtggcaccgagtcggtgctttttttgaagcttctcgagatctccatggacgcgtgacgtcgactcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttattaatagaaactgtagaggtatttttataatttaaaagatgttaaagagtgaggagtaattttgttctaacgcctcactcttttcattttatgattaaatgtatgctgatttacgctaacttaaatcctaaataataacctaatgttaatattttgtaacaaatggataaaagcgtaaaaatattattgtaataattttaagtaggtttaaaatatatataatgtagaagcattcctacattatattatttaaataataatctaaacaggaggggttaaagtggttgatttcaaatctgtgtaaacctaccggggtttgggcgtagccattatattcatgaactccaagaaagcagtatgctagcaaagaaataaaactcaaagcagagagaaaatttagacattcaactataaataaaaaataccccccaaagcattaatatcttggggagtattttttattttgaagtattctgttcagctaaatattcttctaaggtaatacctctgttcataatttcttgtgaggcaggaagaccgatatatcttacatgccatggctcaaaattatactttgttatgttttctttatccttaggatatcttattatgaaaccatatttaccacaattttgttgaagccatttataagaatttgtattcataaatccatcatctaaagaagagtattcggttgatagtaagtccattgccaatccagtttgatgctcacttgtaccaggttcagctacatatttatcagcttcggcttttccgtctcgtgctactttttcattatataatttttgctgatacgaataaggtctataacctgaaacagctagaagtgtaagaccatcctttgatgctgcattaaacatattttcaagtcctgttgcagcttcgctctccatttgatttacattaggatcagaactactaataaatttaacgttaggagttctcaaattttgaggtatatagtttcctgataatttactttgcttgtttacaagtaggatgttctgtttctttacctcgggtttcttggcttgttttttaggtgtagaaactttctttttgggttcgtttg

SEQ ID NO: 80 - DNA, 6560 bp, Artificial Sequence
  pGRNA_deltabdhA_deltabdhB
gatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctaggatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagactctatcattgatagagtttgaaactctatcattgatagagtataatatctttgttcatgcttattacgacataacacagttttagagctagaaatagcaagttaaaataaggctagtccgttatcaacttgaaaaagtggcaccgagtcggtgctttttttgaagcttctcgagatctccatggacgcgtgacgtcgaccttctaatctcctctactattttagggttagctacattagctaaataggtaatagctacagttgtctttgaattctcacctaaagtaagttcttccactttaaaatcagtgcttctaattttttttcttaaaagggctacatttgtggttaaagattcagtgaagccctctctaggacctcttattacagtttcaacagttggttctgttatagctctttcagggggttttccaatacttataataattgctttactttcaccatctaggaataatgctatacttccttttaaaatggacaatataacatcatccatgcttttatatacatttttatcattaacagcaaaaattgattttgtatattcaaatatgtttaaatggggatggttattgtaatcttcttctataagtttttttataacagaggattctattacatcagattggataagattatttatgtagacaatcattgcagaaaaatttctattattagctattttaaattctctaatcgttaaatctgagcaatttgtaaataaggtttctatagtatgtttatttgttttaaggctagttgaaaccgtcttcgcgttatttttagatgcttcttctttattaaaaattttattaaacaacgaaaaattcaccccctcaatttatttatataatagtagtttgcatgaaatttcgttgtttattcatattagatgcttgtattaaaataataaaatagtaaaatataagtagacaaactataaatctattactaggaggtaagaagtatgctaagttttaaatctgtgtaaacctaccggggtttgggcgtagccattatattcatgaactccaagaaagcagtatgctagcaaagaaataaaactcaaagcagagagaaaatttagacattcaactataaataaaaaataccccccaaagcattaatatcttggggagtattttttattttgaagtattctgttcagctaaatattcttctaaggtaatacctctgttcataatttcttgtgaggcaggaagaccgatatatcttacatgccatggctcaaaattatactttgttatgttttctttatccttaggatatcttattatgaaaccatatttaccacaattttgttgaagccatttataagaatttgtattcataaatccatcatctaaagaagagtattcggttgatagtaagtccattgccaatccagtttgatgctcacttgtaccaggttcagctacatatttatcagcttcggcttttccgtctcgtgctactttttcattatataatttttgctgatacgaataaggtctataacctgaaacagctagaagtgtaagaccatcctttgatgctgcattaaacatattttcaagtcctgttgcagcttcgctctccatttgatttacattaggatcagaactactaataaatttaacgttaggagttctcaaattttgaggtatatagtttcctgataatttactttgcttgtttacaagtaggatgttctgtttctttacctcgggtttcttggcttgttttttaggtgtagaaactttctttttgggttcgtttg

SEQ ID NO: 81 - DNA, 1654 bp, Artificial Sequence
  bgaR acrIIA4 cassette
aaaaagtataacagaggttttaatttacgcctctgttatactttttatttttgaaatttttttgttttaaagctgtattttaaatttatatacttggtttatttacttgattatttctgtaatttagtggagacattgaaaaatgttttgaaaaagtttttgaaaataacagggagtcactataacctacactacttgcgacttctcctataggaagtttagtgctttttaataaaagggtggctttgtacattctaaggtttattaaatatctttgaggagaaattccaaggttttttatgaacattttatataaataacttctacttaagttcacataatcagcaatttcttgaacagttatgctatgcatgtaattagaattaatgaaattaagagcatcttgaatatatgtgtgtaattccttatctttgtattcaaaaggttttgggaattcttctataagtgcgtacaataatgagtaaagttcttttagtaatagtatgtcatcagatcttgaaggattataagtttttgatatttcgcacatatttaatattatctgtggaatttttgagttttcttcacaattagcaacacaggagttagtaatagaagttctatttaaatactcattagcatttgaaccactaaatcctatccagtagtattcccaaggatcatcaatagaagccacatactcaacttgcataccttttagtagtataaaaatatcaccttgttttaagttatataccttaccattaaatttaaaagttccatatcccttagttacgtaatgaataacagcatttttcaatacttcatagttatatcctaatcctggtataccttgttctataccacattcatctacattcatttcaaagttttctttaacatactttttccacaatatttgcatttctacctcctaacctataaaattagccaattttatagtagtcttatattaaacatttacatgagagctttgcaaagcagtttatcaacataaaagctttttattttaaaataaattcttctaaatataagaatattttaaagaaatatctttatatattagttattaaaatttataagattataagaaacattataacatattttagaactttttaactattctaaaagattaatttacatattaacatttaattatgggtaaaaactattttgaaaaatgatttatatggaattatgtttcttaaatatacaatcatgtttcatgaatacataattattttaaatgtattgggagggtaaaatgatattaaaaaatgaataccatgaagatactgcagaatctagaatccgcggtagtcgacgtggaattgtgagcggataacaatttcacaggagggctgaaatgaatattaatgacttaattagagaaataaaaaacaaagattacacagtgaaattgagtggtacggatagcaatagtataacacagctaattattagagttaataatgatggaaacgagtatgtaatttctgaaagtgaaaatgaatcaatagttgaaaaattcatatctgcatttaaaaacggttggaatcaagaatacgaggatgaagaagaattttataatgacatgcaaacaatcaccttaaaaagtgagttgaactaa

SEQ ID NO: 82 - DNA, 4984 bp, Artificial Sequence
  pGRNAind
caagcttcaaaaaaagcaccgactcggtgccactttttcaagttgataacggactagccttattttaacttgctatttctagctctaaaacagagaccgctagcgatatccccgggagatctggtctcaatgaacaaagatattatactctatcaatgatagagtttcaaactctatcaatgatagagtgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctaggatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagatctccatggacgcgtgacgtcgactctagaggatccccgggtaccgagct

SEQ ID NO: 83 - DNA, 200 bp, Artificial Sequence
  gRNA cassette
gagctcactctatcattgatagagtttgaaactctatcattgatagagtataatatctttgttcattgagaccagatctcccggggatatcgctagcggtctctgttttagagctagaaatagcaagttaaaataaggctagtccgttatcaacttgaaaaagtggcaccgagtcggtgctttttttgaagcttgagctc

SEQ ID NO: 84 - DNA, 24 bp, Artificial Sequence
  primer
tcatgatttctccatattagctag

SEQ ID NO: 85 - DNA, 24 bp, Artificial Sequence
  Primer
aaacctagctaatatggagaaatc

SEQ ID NO: 86 - DNA, 24 bp, Artificial Sequence
  Primer
tcatgttacacttggaacaggcgt

SEQ ID NO: 87 - DNA, 24 bp, Artificial Sequence
  Primer
aaacacgcctgttccaagtgtaac

SEQ ID NO: 88 - DNA, 24 bp, Artificial Sequence
  Primer
tcatttccggcagtaggatcccca

SEQ ID NO: 89 - DNA, 24 bp, Artificial Sequence
  Primer
aaactggggatcctactgccggaa

SEQ ID NO: 90 - DNA, 24 bp, Artificial Sequence
  Primer
tcatgcttattacgacataacaca

SEQ ID NO: 91 - DNA, 24 bp, Artificial Sequence
  Primer
aaactgtgttatgtcgtaataagc

SEQ ID NO: 92 - DNA, 36 bp, Artificial Sequence
  Primer
atgcatggatccaaacgaacccaaaaagaaagtttc

SEQ ID NO: 93 - DNA, 30 bp, Artificial Sequence
  Primer
ggttgatttcaaatctgtgtaaacctaccg

SEQ ID NO: 94 - DNA, 30 bp, Artificial Sequence
  Primer
acacagatttgaaatcaaccactttaaccc

SEQ ID NO: 95 - DNA, 37 bp, Artificial Sequence
  Primer
atgcatgtcgactcttaagaacatgtataaagtatgg

SEQ ID NO: 96 - DNA, 36 bp, Artificial Sequence; identical to SEQ ID NO: 92
  Primer

SEQ ID NO: 97 - DNA, 30 bp, Artificial Sequence
  Primer
gctaagttttaaatctgtgtaaacctaccg

SEQ ID NO: 98 - DNA, 32 bp, Artificial Sequence
  Primer
acacagatttaaaacttagcatacttcttacc

SEQ ID NO: 99 - DNA, 37 bp, Artificial Sequence
  Primer
atgcatgtcgaccttctaatctcctctactattttag

SEQ ID NO: 100 - DNA, 20 bp, Artificial Sequence
  Primer
acacattgaagggagctttt

SEQ ID NO: 101 - DNA, 20 bp, Artificial Sequence
  Primer
ggcaacaacatcaggccttt

SEQ ID NO: 102 - DNA, 4966 bp, Artificial Sequence
  pGRNA-xylB
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagaagcttcaaaaaaagcaccgactcggtgccactttttcaagttgataacggactagccttattttaacttgctatttctagctctaaaacctagctaatatggagaaatcatgaacaaagatattatactctatcaatgatagagtttcaaactctatcaatgatagagtctcgagatctccatggacgcgtgacgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 103 - DNA, 4966 bp, Artificial Sequence
  pGRNA-xylR
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagactctatcattgatagagtttgaaactctatcattgatagagtataatatctttgttcatgttacacttggaacaggcgtgttttagagctagaaatagcaagttaaaataaggctagtccgttatcaacttgaaaaagtggcaccgagtcggtgctttttttgaagcttctcgagatctccatggacgcgtgacgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 104 - DNA, 4966 bp, Artificial Sequence
  pGRNA-glcG
agctcggtacccggggatcctctagagtcgacgtcacgcgtccatggagatctcgaggcgtgctagcttataatataatattgtggaatattgaaattaattttactaaaaactattccaatcaaggcagcaataataaaaatagttttatttattggtttagagtacgtgtttttatttttaagtttctttttaatagctgttagaaagaaacttaaaaataaaatatatataataataaatataataaaaatatatataaatttagaaaaagataatatttgataaaacatatttaaagatataatcaaatcctgtaaagatgaaaatgaaaataaaattcctaaaaataagtttaataaagccggaaatttcactagaaatttcactagtttttttaaaaacaataaatataataaagatattattataaaacttaaatttaataataacatcataaggaaaatggattggtttacttcaacggctttattatatgaaaaaaatacaattaaatttaaacataatattacaaaaagtattgttaaaattgaaaaaaattaaatctttagtatctttggaaataatcataatttataagctcctttgattttttatataaattatacattaattatttaatatataaaaactaaaattagtaaatcatctaaatattacaaatgaagaaatattatttgtaaataattatagtaatatattatggaatgaaatagtaaaaaattaaaaagaatcatttaatttagtttaaaatattttaataaaaataataaattaaaacataaaaaatagattaagtttcaattggggacttactctattttttattaagagtttaagaatagttaataattattgaaaatatgatataattttcttagttttacagaaggggaggtgaataagtgagaaacgcattaaagctgaataaaaagaactatatagataatattcatagtagaagtaaagggtggataaccaggagcgttatagataaaaaaggatatagccaatggcactataaatacgctgaattaaaagatttagatatgagtgatgaaaatatctatataactctaaataccttttataagccgtgtaggcgattagaaaatataaaagagttaaatacactgtttatagacttggattattataaaactggcaaaactaaagaccaggtattaatggacttagaaaagaattattttaatcaaagtattcctataccaaactatgtaatagatagtggaagaggaatgtatttaatatggataataaatgcagtacctagtaaagcattaccattatggaaagcggttcaagaatatttatataatcaattaaaatactttggagcagatagacaagccttagatgcaacgagaatattaagagttccaggaagtataaactctaaatcaaaaacagtagtcaatatattagatgagtacgaatatatttatgacttaagagaaatacaaaatggatttttacctgaattaaaaccatatgaaaggaaaaagggtagaccaagcaaaataaattatatttatagagaaagaagtttatattatggaagaatacaagacataataaaactttgtgaactaagagaatatgatttaaaaggacacagagagcttatattatttttatatagatattatctttgtagctttacagaagacattgagaaggcattaaatgatgttttagaacttaatagtatgtttagacaacatttaagtgaaagagaagttataagagcaactagaagtgctgaaagatgttatttagataaaaataagcaatataagtataagaatgaaactcttatagagttattagaaattacagaagaagaacaaaaatatatgacaataataatttctaaaaaggaatataagagaagagaaaatattagaggtaaaaaaaattatcaagagcaattaaaagctaaaggaaaagcaacaaaaaaagaggaattaaatgtattaagaaaaaaaataaaagcccttaaagaaaaaggctttaaaaataaagaaattactctaatgttagaagtaccaataaaaacattagaacgtcatattacgtatatgaaaaaaaatgggcttttataaaggctcattttttatattcttttcttcaaagattatataatataaaaaaatttttttcaaactttaaataaaaaatatttttatatttttttatttttttatttttatatttttttatttttttatttttatatttttttatttttatatttttttatttttatatttttttatttttttatttttttatttttttatttttttatttttttatttttttatttttaccctcattttttttacgcttgtattatagggtactttgtacctgttcttttttttggggaggttgtaaagataattttttactttagttagttccgaaggaacttttattatagtgcccctttatttttttgcgtggcattgaatgtaaaaaattatcactatactagggcgtaaagtaatattacatgtgtctcaaagtgggattaaagcgggattttatagggcgtgtttgtggcttagagtgggattattggaaatttttttgatcctaggttaaatactagtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtaggtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccggggatcgatccccggccgagcgcttagtgggaatttgtaccccttatcgatacaaattccccgtaggcgctagggacctctttagaccggtcagctgtcagacacttatcacattaagtatatactagtattaagctagctttggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcactctatcattgatagagtttgaaactctatcattgatagagtataatatctttgttcatttccggcagtaggatccccagttttagagctagaaatagcaagttaaaataaggctagtccgttatcaacttgaaaaagtggcaccgagtcggtgctttttttgaagcttg

SEQ ID NO: 105 - DNA, 4938 bp, Artificial Sequence
  pGRNA-bdhB
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagtatattgataaaaataataatagtgggtataattaagttgttaggaggttagttagagcttattacgacataacacagttttagagctagaaatagcaagttaaaataaggctagtccgttatcaacttgaaaaagtggcaccgagtcggtgctttttttgaagcttgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 106 - DNA, 4790 bp, Artificial Sequence
  pEC750C
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagatctccatggacgcgtgacgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 107 - DNA, 35 bp, Artificial Sequence
  Primer
acttgggtcgaccacgataaaacaaggttttaagg

SEQ ID NO: 108 - DNA, 42 bp, Artificial Sequence
  Primer
taccagggatccgtattaatgtaactatgatatcaattcttg

SEQ ID NO: 109 - DNA, 46 bp, Artificial Sequence
  Primer
atgcatggtcccaatgaataggtttacacttactttagttttatgg

SEQ ID NO: 110 - DNA, 39 bp, Artificial Sequence
  Primer
atgcgagttaacaacttctaaaatctgattaccaattag

SEQ ID NO: 111 - DNA, 47 bp, Artificial Sequence
  Primer
atgcatggatcccaatgaataggtttacacttactttagttttatgg

SEQ ID NO: 112 - DNA, 39 bp, Artificial Sequence
  Primer
atgcgagagctcaacttctaaaatctgattaccaattag

SEQ ID NO: 113 - DNA, 32 bp, Artificial Sequence
  Primer
atgcatggatccgtctgacagttaccaggtcc

SEQ ID NO: 114 - DNA, 39 bp, Artificial Sequence
  Primer
atgcgagagctccaattgttcaaaaaaataatggcggag

SEQ ID NO: 115 - DNA, 32 bp, Artificial Sequence
  Primer
atgcatggatcccggcagtttttctttttcgg

SEQ ID NO: 116 - DNA, 40 bp, Artificial Sequence
  Primer
atgcgagagctcggttaaatactagtttttagttacagac

SEQ ID NO: 117 - DNA, 2686 bp, Artificial Sequence
  pUC19
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 118 - DNA, 4282 bp, Artificial Sequence
  pNF2
ctggagaggattgtccttatacttatcataagcatgaaggacttgttattcctagatagagaattaattatgttaaagagatataataaactcattataattataatttttagtataattattattgcaattttttcgtataaatatctaataatgccaaaagagcatagaatagaaatttcaacattatcaaacatagaagtttttaaatttaatagtttttcaaagtttagtaacgaaaaaatgtatactattaatgatagtgataagttaataaaattcaaaacactatttaataatttagataaatcaaaagatataaaaaagattagtattccggaaagtgaaaatttaaatgcatttaaattttctgcacatataaaacttaactttaactatgttaataaagatagccaaataactgaaggtgcttttcttatgtatattttggtagacaatttagaagggaagtcatatatgacttttttaggacaagattcaagctatatattagatagtaatgaaactaacattttaagagaaatatttatgaattcagagattaattaatttatgaattcataaatattatctaagcacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatacatttattttgaagggggaaaatgttttatgaaaagactacttaaactacctattttatcattattaggattatttttaattggatcaactccaacattagctttaactaaagataataatcaaaatttagatactatgaaagtaaacttatatactgaaacagtagatgtgtttgataaagatgcatttaaacaaacatttactaataaagatataaaatttctagaggattctttgaatgcaaaaataaattattcaggtaaatctgttacagtaacaatgaaaaacaaaattaagccatctactaaacaagggcttgttttatatgtaaatggaaaatcagttaatgttgattcagatggcagtataaaagtacctaaagatactaagaaaatttctaaattaaataaagataaatcaatgatggatggatcaatgatggataaatcattacatgatgagaattgtgtagtatcagatagtttttataatgctgatgttaataatataaattcaaaagaagcagaagctgtatttaaagtaagttctggtgaattattagctaaaatggatgaaaaagaagatgattacatacaaaagaactcatctaaaattctagcagctgcttatcataagggatatggggacaagtactatgaaggagattgggttcattgcaataggtttaatggtcaacttacagatgatgttcactataattggagaactggaagtgtttcagaaaaagcagctgcaatgagaaatttttatggcagtgattgtcatatagcattagttcaagcaggtagtggatgtacaagtataggttcatgcgaatgcaatacagatcaaatagctgcgtattgttcaggtttcgtaaaagataaaaata

SEQ ID NO: 119 - DNA, 5473 bp, Artificial Sequence
  pNF3
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatacggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 120 - DNA, 9128 bp, Artificial Sequence
  pMTL007S-E1
gatcgggccccctgcagggtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaagcttgagctcataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctaataaagatcttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgtgcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcataccagcaccagaagcaccagcatctcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgccatatgaatccctcctaatttatacgttttctctaacaacttaattatacccactattattatttttatcaatataacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcaccatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatgtcgagcacccgttctcggagcactgtccgaccgctttggccgccgcccagtcctgctcgcttcgctacttggagccactatcgactacgcgatcatggcgaccacacccgtcctgtggatcgccaagccgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacgggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggcccaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagacc

SEQ ID NO: 121 - DNA, 5002 bp, Artificial Sequence
  pEC751S
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagatctccatggacgcgtgacgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtcccaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagttgttaacttcaggtttgtctgtaactaaaaactagtatttaacctagg

SEQ ID NO: 122 - DNA, 3907 bp, Artificial Sequence
  pFW01
tcgagatctccatggacgcgtgacgtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggc

SEQ ID NO: 123 - DNA, 6525 bp, Artificial Sequence
  pNF3S
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatacggatcccaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagttgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 124 - DNA, 6554 bp, Artificial Sequence
  pNF3E
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatacggatccgtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 125 - DNA, 6271 bp, Artificial Sequence
  pNF3C
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatacggatcccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 126 - DNA, 2793 bp, Artificial Sequence
  OREP
cacgataaaacaaggttttaaggataagaaaagtcatgagatttatagtaaatcttgtgactttttttattgaatagtagagagagttcggaagtataacacgctatattcttgatatttttagaatagcaagcattggatttgtcctgacactttcccaaaaattaaggagttattccttaaaccaaaaagattaatgtgggaacaaatttagtgtatccatttttgaagggcgcacttatacaccaccaaaatggtgtgtgcgaaatctttaaaaaagatttatcaaaaagcttttttaaagctgggacatttagaaaatcaataatgttttttgcccaatacgctagtcttaaaatctgcaaggttgataactatttagtcccaggtattagaatggggcatatatatacaaagtatatatatgcgtaaatatatgtgggactgtgggaacaaaattgcgtgctaaaattgtattgaaaaggtaatgaaaaggtcatgctttggtattgctaacgtatagaaaaggtaatgaaaagctcatggttctataaaaaagatgtacccacgaaaataataggctttgcctatttccccatgtaatatgggggcagttttctcttatgctctttcttaacatattgaataaatacaaaatgcagctttgtgggaataaaaatatttttgtttttattcttatagttagacaaaattttaatcttttttgtgctataacaagattaaaatttgtgggaacattaagaaatattgttgtcacaaataaaaaggagagtgggaacaattgctataaaaaacgcagaaattaagattagagttacaaaagagcaaaaagaattatttaagaaaattgcaaaagctgaaaatatgagtatgagtgaatttattattgtgaccacagaatatttagccagaaaaaaagatgaaaatatgaaatcaaaagacatgatcgagagaagagctgcgaagactgaagaaaaaattatgaagctaaaaaagaaactaaataaaaacaggtaatatagattacagttttaagcttgttttccctatagactagagtaaatatataaatatacctgtcaagggcttataagcccctttagggggtgcgtagcacccttgacaggtatatttatatattttagggtgccattaagggaaacaagctttaaaatgcctttaaaggcattttaaaataaataaaaaaaagatggtttttaccatcttttttaactcccgaaagggagttctttcttttcttgatactatacgtaactatttcgatttgccctgaacctaatcaaagctagataaattcagtattagggcataaaaaaacttgctttttcgggtggaaatctgtataatttaaattgcttagataaaaattaccaattccatacgaaaggagcaagttttacataaggttaaagccttatgtgaattctcatttaattacatgaataataataacacagaaagtgaagaattaaaagagcaaagtcaactattgcttgacaaatgcacaaaaaagaaaaagaaaaatcctaaatttagtagttatatagaaccattagtaagcaagaaattatctgaaagaataaaggaatgtggtgactttttgcagatgttatctgatttaaaccttgaaaattcgaaactgcatagagcaagtttttgtggtaacagattttgtcctatgtgtagctggcgtattgcttgtaaggatagtttggaaatatctattctcatggagcatttacgcaaagaggaaagcaaagaatttatctttttgaccttaacaactccaaatgtgaaaggtgcggaccttgataattccataaaagcatacaataaagcatttaaaaagttaatggaacgcaaagaggtcaagagcatagtaaaaggctacataagaaagctagaagtaacctataatttggacaagagttccaaatcatataatacttatcacccacatttccatgtggtactagcagtcaatagaagttactttaaaaagcaaaatctatatataaaccatcatagatggcttagtttgtggcaagagtcaactggtgattattcgataactcaagttgatgtaagaaaggctaaaattaacgattataaagaggtttatgagcttgctaagtattcggctaaggattccgactatttaatcaatagagaagtgtttacggtattctacaaatctttaaagggtaaacaggtacttgtatttagtggattatttaaagacgctcataaaatgtataagaatggagagctagatctgtataagaagttggatactatcgaatatgcttatatggtaagttataactggcttaaaaagaagtatgatacttcaaatattagagaattaactgaggaagaaaagcagaaattcaataaaaatttaatcgaagatgtggatattgagtaggtgggattatatctcaccttttttattgtcttttcatgttgaaattttgacgcttaatgcatgaagtattgacaagtttaaaaattacggtttttaatccttagttgattagcaggattatggccggaatgctccgtccagtcctgttaaggaattaaaattccctaaaacccttggctatgatttatagcgagaatcgtcaattaaaaatttaataggtgctatgaaagtcgattaataattaattttaaaatgcaatatgaaacataattacaagaatttgacttttaatacaagaattgatatcatagttacattaatac

SEQ ID NO: 127 - DNA, 2793 bp, Clostridium beijerinckii; identical to SEQ ID NO: 126

SEQ ID NO: 128 - protein, 329 aa, Clostridium beijerinckii
MNNNNTESEELKEQSQLLLDKCTKKKKKNPKFSSYIEPLVSKKLSERIKECGDFLQMLSDLNLENSKLHRASFCGNRFCPMCSWRIACKDSLEISILMEHLRKEESKEFIFLTLTTPNVKGADLDNSIKAYNKAFKKLMERKEVKSIVKGYIRKLEVTYNLDKSSKSYNTYHPHFHVVLAVNRSYFKKQNLYINHHRWLSLWQESTGDYSITQVDVRKAKINDYKEVYELAKYSAKDSDYLINREVFTVFYKSLKGKQVLVFSGLFKDAHKMYKNGELDLYKKLDTIEYAYMVSYNWLKKKYDTSNIRELTEEEKQKFNKNLIEDVDIE

SEQ ID NO: 129 - protein, 256 aa, Artificial Sequence
  Consensus COG5655
MCQKRSDYSDEKAWLKDKSKDGKVEPWREKKEANVKYFELLKILMFKKAERVYRCNELLELQKVNETGENKLCPNWFCKSLLCPMCNWRKPMKSDLQDGLYVKRVISYGPLLKWKHLKLNLKNVEDGDLLNKSLDEMALGFKRTMGFKKIAKNFVGFMKSTEITYNEKDNSYNQHMHVLFCSEQTYFKNFINNTPQEFWNKRWSKAMKLDYDPQVMKLWTMYKKEIKNYIQTALQETAKYDVKDMDSATIDDEKSL

SEQ ID NO: 130 - DNA, 768 bp, Enterococcus faecalis
gtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataa

SEQ ID NO: 131 - DNA, 738 bp, Clostridium difficile
atgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 132 - DNA, 3792 bp, Artificial Sequence
  Optimized Mad7 CDS for B. subtilis
atgaacaacggcacaaataattttcagaactttattggcatttcatcattgcagaaaacgttaagaaatgctttaattccgacggaaacaacgcaacagtttattgttaaaaacggaattattaaagaagatgaattaagaggcgaaaacagacagattttaaaagatattatggatgactactacagaggatttatttctgaaacattatcatctattgatgacattgattggacaagcttatttgaaaaaatggaaattcagttaaaaaatggtgataataaagatacattaattaaagaacagacagaatatagaaaagcaattcataaaaaatttgcgaacgacgatagatttaaaaacatgtttagcgccaaattaatttcagacattttacctgaatttgttattcataacaataattattcagcatcagaaaaagaagaaaaaacacaggtgattaaattgttttcaagatttgcgacaagctttaaagattactttaaaaacagagcaaattgcttttcagcggacgatatttcatcaagcagctgccatagaattgttaacgacaatgcagaaatttttttttcaaatgcgttagtttacagaagaattgtaaaatcattaagcaatgacgatattaacaaaatttcaggcgatatgaaagattcattaaaagaaatgtcattagaagaaatttattcttacgaaaaatatggcgaatttattacacaggaaggcattagcttttataatgatatttgtggcaaagtgaattcttttatgaacttatattgtcagaaaaataaagaaaacaaaaatttatacaaacttcagaaacttcataaacagattctgtgcattgcggacacaagctatgaagttccgtataaatttgaatcagacgaagaagtgtaccaatcagttaacggctttcttgataacattagcagcaaacatattgttgaaagattaagaaaaattggcgataactataacggctacaacttagataaaatttatattgtgtccaaattttacgaaagcgttagccaaaaaacatacagagactgggaaacaattaatacagccttagaaattcattacaataatattttgccgggtaacggtaaatcaaaagccgacaaagtaaaaaaagcggttaaaaatgatttacagaaatccattacagaaattaatgaactggtgtcaaactataaattatgctcagacgacaacattaaagcggaaacatatattcatgaaattagccatattttgaataactttgaagcacaggaattgaaatacaatccggaaattcatctggttgaatccgaattaaaagcgtcagaacttaaaaacgtgttagacgtgattatgaatgcgtttcattggtgttcagtttttatgacagaagaacttgttgataaagacaacaatttttatgcggaattagaagaaatttacgatgaaatttatccggtaatttcattatacaacttagttagaaactacgttacacagaaaccgtacagcacgaaaaaaattaaattgaactttggaattccgacgttagcagacggttggtcaaaatccaaagaatattctaataacgctattattttaatgagagacaatttatattatttaggcatttttaatgcgaaaaataaaccggacaaaaaaattattgaaggtaatacgtcagaaaataaaggtgactacaaaaaaatgatttataatttgttaccgggtccgaacaaaatgattccgaaagtttttttgagcagcaaaacgggcgtggaaacgtataaaccgagcgcctatattctggaaggctataaacagaataaacatattaaatcttcaaaagactttgatattacattttgtcatgatttaattgactactttaaaaactgtattgcaattcatccggaatggaaaaactttggttttgattttagcgacacatcaacatatgaagacatttccggcttttatagagaagtagaattacaaggttacaaaattgattggacatacattagcgaaaaagacattgatttattacaggaaaaaggtcaattatatttatttcagatttataacaaagatttttcaaaaaaatcaacaggcaatgacaaccttcatacaatgtacttaaaaaatcttttttcagaagaaaatcttaaagatattgttttaaaacttaacggcgaagcggaaattttttttagaaaaagcagcattaaaaacccgattattcataaaaaaggctcaattttagttaacagaacatacgaagcagaagaaaaagaccagtttggcaacattcaaattgtgagaaaaaatattccggaaaacatttatcaggaattatacaaatactttaacgataaaagcgacaaagaattatctgatgaagcagccaaattaaaaaatgtagtgggacatcatgaagcagcgacgaatattgttaaagactatagatacacgtatgataaatactttcttcatatgcctattacgattaattttaaagccaataaaacgggttttattaatgatagaattttacagtatattgctaaagaaaaagacttacatgtgattggcattgatagaggcgaaagaaacttaatttacgtgtccgtgattgatacatgtggtaatattgttgaacagaaaagctttaacattgtaaacggctacgactatcagattaaattaaaacaacaggaaggcgctagacagattgcgagaaaagaatggaaagaaattggtaaaattaaagaaattaaagaaggctacttaagcttagtaattcatgaaatttctaaaatggtaattaaatacaatgcaattattgcgatggaagatttgtcttatggttttaaaaaaggcagatttaaagttgaaagacaagtttaccagaaatttgaaacaatgttaattaataaattaaactatttagtatttaaagatatttcaattacagaaaatggcggtttattaaaaggttatcagttaacatacattcctgataaacttaaaaacgtgggtcatcagtgcggctgcattttttatgtgcctgctgcatacacgagcaaaattgatccgacaacaggctttgtgaatatttttaaatttaaagacttaacagtggacgcaaaaagagaatttattaaaaaatttgactcaattagatatgactcagaaaaaaatttattttgctttacatttgactacaataactttattacgcaaaacacggttatgagcaaatcatcatggtcagtgtatacatacggcgtgagaattaaaagaagatttgtgaacggcagattttcaaacgaatcagatacaattgacattacaaaagatatggaaaaaacgttggaaatgacggacattaactggagagatggccatgatcttagacaagacattattgattatgaaattgttcagcatatttttgaaatttttagattaacagtgcaaatgagaaactccttgtctgaattagaagacagagattacgatagattaatttcacctgtattaaacgaaaataacattttttatgacagcgcgaaagcgggcgatgcacttcctaaagatgccgatgcaaatggtgcgtattgtattgcattaaaaggcttatatgaaattaaacaaattacagaaaattggaaagaagatggtaaattttcaagagataaattaaaaattagcaataaagattggtttgactttattcagaataaaagatatttataa

SEQ ID NO: 133 - DNA, 10469 bp, Artificial Sequence
  pCas9cond
catggataaaaagtacagtattggtctagacataggaactaactctgttgggtgggctgttataacagatgaatataaagttccatcaaaaaaatttaaagtattaggaaacactgatagacattcaataaaaaaaaacttgataggtgctttattattcgattcaggagagactgctgaagctacacgtttaaaaagaacagctagacgtagatatacaagaagaaaaaataggatatgttatcttcaagaaatttttagtaatgaaatggcaaaagttgatgattcattctttcacagactagaagaaagtttcttagttgaagaagataagaagcatgaaagacaccctatttttggtaatatcgtagatgaagtagcatatcatgagaagtatccaactatctatcatttaagaaagaaattagttgattctacagataaagctgatctgagattaatatatttagctttagctcatatgattaaatttagaggacattttttaatagaaggtgatttaaacccagacaacagcgatgtagataaattatttatccaattagttcaaacttataatcaattattcgaagagaatccaattaatgcaagtggtgtagacgctaaggctatattatcagctagattatcaaaatctagaagattagaaaatctaatagctcaacttcctggagaaaagaaaaatggactttttgggaacctaatagctctctcactcggactaacaccaaattttaaaagcaattttgatcttgctgaagacgcaaagttacaactatcaaaggatacatacgatgatgatttagataatttgttagctcaaataggtgatcaatatgctgatttgtttcttgcagcaaaaaacttaagtgatgcaattttactatcagatatacttagagtaaatacagaaataacaaaggctcctttatcagcaagtatgattaaacgatatgatgagcatcatcaagatttaacattattaaaggcacttgtaagacaacaattaccagaaaaatataaagaaattttctttgatcaatctaaaaatggatatgctggatatatagacggtggagcaagtcaagaagagttttataaatttataaagcctattttagaaaaaatggatggaactgaagaattacttgttaaacttaacagagaagatttacttagaaaacaaagaacttttgataatggttcaattcctcaccaaattcatttaggagaattacatgctatactaagaagacaagaagatttttatccatttcttaaagataatagagaaaaaattgaaaaaattttaacttttagaataccatattatgtaggaccacttgcaaggggaaattcaagatttgcatggatgactagaaaatcagaagaaactataaccccgtggaattttgaagaagtagtagataaaggagctagtgctcaatcatttatagaaagaatgacaaattttgataagaatcttcctaacgaaaaggttttgccaaagcatagccttctttatgagtattttacagtttataatgagcttactaaagtaaaatacgttacagaaggaatgagaaaaccagcatttttgtctggtgaacaaaagaaagcaatagtagacctattatttaaaacaaataggaaggttaccgtaaagcaacttaaagaagattacttcaaaaaaattgaatgctttgatagtgttgaaatatcaggagttgaagatagatttaatgcttcacttggtacatatcacgatctcttaaaaattataaaagataaggattttttagataatgaagaaaatgaagatattcttgaagatatagtattaacattgacactttttgaagatagagaaatgatagaagaaagattaaaaacatatgcacatctttttgatgataaggttatgaagcaacttaaaagaagaagatatacaggttggggacgtttgtcaagaaagctaattaatggtattagagataaacaatcaggaaagactattctcgattttcttaaatcagatggatttgctaatagaaactttatgcaattaattcatgatgattctcttactttcaaagaggatattcaaaaggctcaagtttctggacaaggcgatagcttacacgaacacattgctaaccttgcagggagccccgctatcaaaaaaggaattttacaaacagttaaagttgtagatgaacttgttaaagttatgggaagacacaaacctgagaatatagttatagaaatggccagagaaaatcaaacaacacaaaaaggacaaaaaaattctagagagagaatgaagagaattgaagaaggaataaaagagctaggatcacaaatattaaaagaacatccagttgaaaatactcaattgcaaaatgaaaagttatatttgtattacttacaaaatggaagagatatgtatgttgatcaagaactcgatattaatagattaagtgactatgatgttgatcatattgttcctcaatcatttttaaaagatgattcaatcgataacaaagtattaactagatcagataaaaatagaggaaagtcagataatgtaccatctgaagaagttgttaaaaaaatgaagaactattggagacaacttttaaatgcaaagctaattacacaaagaaaatttgacaatttaacaaaagcagaaagaggaggattaagcgaattagacaaagctggatttataaaaagacaacttgttgagacaagacaaataactaagcatgttgctcaaatacttgattcaagaatgaatacaaaatatgatgaaaatgataaattaatcagagaagtaaaagtaataacattaaagtcaaaattagtatcagatttcagaaaggattttcaattttacaaagttcgtgaaataaataactatcatcatgctcatgatgcatacttaaatgctgttgtaggaactgctcttattaagaaatatcctaaactagaaagcgaatttgtttatggagattataaagtttatgatgtgcgcaaaatgatcgcgaaatccgaacaagaaatcggtaaggctacagcaaaatatttcttttatagtaatataatgaatttttttaagacagaaataactttggctaatggtgaaatcagaaaaagaccacttatcgaaacaaatggagagacaggagaaatagtatgggataaaggaagagattttgctactgttagaaaagtactaagtatgccacaagtaaatatcgtaaagaaaactgaagttcaaactggaggtttctctaaggaatcaattttacctaagagaaattcagataagttaattgcaaggaaaaaagattgggacccaaaaaaatacggtggttttgatagtccaacagttgcctatagtgttcttgtagtagcgaaagttgagaaaggtaagtcaaaaaagttgaaaagcgtaaaagaacttcttggtatcacaattatggaaagatcttcatttgaaaaaaatccaattgactttttagaagctaagggttataaagaagttaaaaaggatttaatcataaaactaccaaagtatagtctatttgaactcgaaaacggaagaaaacgaatgctcgctagcgcaggagaacttcaaaaaggaaatgaacttgcgctgccatcaaagtatgtaaatttcttatatttagcttctcattatgagaaattaaaaggatcaccagaggataatgaacaaaagcaactatttgtagaacaacacaaacattatttagatgaaataatagaacaaatatctgaattttctaaaagagttatacttgccgacgcaaatctagataaggtgctttcagcgtataataaacacagagataaaccaataagagaacaagcagaaaacattatccatctttttacattaactaatcttggtgcaccagctgcatttaagtactttgatacaacaatagatagaaaaagatacacatctactaaagaagtattagacgcaactttaatacatcaatctattacagggctttatgaaacaagaattgatttaagtcaactaggcggagattaagtcgacaaagtattgttaaaaataactctgtagaattataaattagttctacagagttattttttgacccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcccaactaactcaacgctagtagtggatttaatcccaaatgagccaacagaaccagaaccagaaacagaatcagaacaagtaacattggatttagaaatggaagaagaaaaaagcaatgacttcgtgtgaataatgcacgaaatcgttgcttatttttttttaaaagcggtatactagatataacgaaacaacgaactgaatagaaacgaaaaaagagccatgacacatttataaaatgtttgacgacattttataaatgcatagcccgataagattgccaaaccaacgcttatcagttagtcagatgaactcttccctcgtaagaagttatttaattaactttgtttgaagacggtatataaccgtactatcattatatagggaaatcagagagttttcaagtatctaagctactgaatttaagaattgttaagcaatcaatcggaaatcgtttgattgctttttttgtattcatttatagaaggtggagtttgtatgaatcatgatgaatgtaaaacttatataaaaaatagtttattggagataagaaaattagcaaatatctatacactagaaacgtttaagaaagagttagaaaagagaaatatctacttagaaacaaaatcagataagtatttttcttcggagggggaagattatatatataagttaatagaaaataacaaaataatttattcgattagtggaaaaaaattgacttataaaggaaaaaaatctttttcaaaacatgcaatattgaaacagttgaatgaaaaagcaaaccaagttaattaaacaacctattttataggatttataggaaaggagaacagctgaatgaatatcccttttgttgtagaaactgtgcttcatgacggcttgttaaagtacaaatttaaaaatagtaaaattcgctcaatcactaccaagccaggtaaaagcaaaggggctatttttgcgtatcgctcaaaatcaagcatgattggcggtcgtggtgttgttctgacttccgaggaagcgattcaagaaaatcaagatacatttacacattggacacccaacgtttatcgttatggaacgtatgcagacgaaaaccgttcatacacgaaaggacattctgaaaacaatttaagacaaatcaataccttctttattgattttgatattcacacggcaaaagaaactatttcagcaagcgatattttaacaaccgctattgatttaggttttatgcctactatgattatcaaatctgataaaggttatcaagcatattttgttttagaaacgccagtctatgtgacttcaaaatcagaatttaaatctgtcaaagcagccaaaataatttcgcaaaatatccgagaatattttggaaagtctttgccagttgatctaacgtgtaatcattttggtattgctcgcataccaagaacggacaatgtagaattttttgatcctaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttaatagggcgtaataacgtcatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagtgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaagtaaaaaaagaagaaaaagaaagctatataaaggcgctgacaaattcttttgacttagagcatacattcattcaagagactttaaacaagctagcagaacgccctaaaacggacacacaactcgatttgtttagctatgatacaggctgaaaataaaacccgcactatgccattacatttatatctatgatacgtgtttgttttttctttgctgtttagcgaatgattagcagaaatatacagagtaagattttaattaattattagggggagaaggagagagtagcccgaaaacttttagttggcttggactgaacgaagtgagggaaaggctactaaaacgtcgaggggcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgattacatggattggattagttcttgtggttacgtggtttttaactaaaagtagtgaatttttgatttttggtgtgtgtgtcttgttgttagtatttgctagtcaaagtgattaaatagaattctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggcctcgagtatattgataaaaataataatagtgggtataattaagttgttaggaggttagttac

SEQ ID NO: 134 - DNA, 8559 bp, Artificial Sequence
  pMAD7
tcgagtccctatcagtgatagattgaaactctatcattgatagagtataatatctttgttcattagagcgataaacttgaatttgagagggaacttagatgaacaacggcacaaataattttcagaacttcatagggatatcaagtttgcagaaaacgttaagaaatgctttaatacccacggaaaccacgcaacagttcatagttaagaacggaataattaaagaagatgagttaagaggcgagaacagacagattttaaaagatataatggatgactactacagaggattcatatctgagactttaagttctattgatgacatagattggactagcttattcgaaaaaatggaaattcagttaaaaaatggtgataataaagataccttaattaaggaacagacagagtatagaaaagcaatacataaaaaatttgcgaacgacgatagatttaagaacatgtttagcgccaaattaattagtgacatattacctgaatttgttatacacaacaataattattcggcatcagagaaagaggaaaaaacccaggtgataaaattgttttcgagatttgcgactagctttaaagattacttcaagaacagagcaaattgcttttcagcggacgatatttcatcaagcagctgccatagaatagttaacgacaatgcagagatattcttttcaaatgcgttagtttacagaagaatagtaaaatcgttaagcaatgacgatataaacaaaatttcgggcgatatgaaagattcattaaaagaaatgagtttagaagaaatatattcttacgagaagtatggggaatttattacccaggaaggcattagcttctataatgatatatgtgggaaagtgaattcttttatgaacttatattgtcagaaaaataaagaaaacaaaaatttatacaaacttcagaaacttcacaaacagattctatgcattgcggacactagctatgaggttccgtataaatttgaaagtgacgaggaagtgtaccaatcagttaacggcttccttgataacattagcagcaaacatatagttgaaagattaagaaaaataggcgataactataacggctacaacttagataaaatttatatagtgtccaaattttacgagagcgttagccaaaaaacctacagagactgggaaacaattaataccgccttagaaattcattacaataatatattgccgggtaacggtaaaagtaaagccgacaaagtaaaaaaagcggttaagaatgatttacagaaatccataaccgaaataaatgaactagtgtcaaactataagttatgcagtgacgacaacataaaagcggagacttatatacatgagattagccatatattgaataactttgaagcacaggaattgaaatacaatccggaaattcacctagttgaatccgagttaaaagcgagtgagcttaaaaacgtgttagacgtgataatgaatgcgtttcattggtgttcggtttttatgactgaggaacttgttgataaagacaacaatttttatgcggaattagaggagatttacgatgaaatttatccagtaattagtttatacaacttagttagaaactacgttacccagaaaccgtacagcacgaaaaagattaaattgaactttggaataccgacgttagcagacggttggtcaaagtccaaagagtattctaataacgctataatattaatgagagacaatttatattatttaggcatatttaatgcgaagaataaaccggacaagaagattatagagggtaatacgtcagaaaataagggtgactacaaaaagatgatttataatttgttaccgggtcccaacaaaatgataccgaaagttttcttgagcagcaagacgggggtggaaacgtataaaccgagcgcctatatactagaggggtataaacagaataaacatataaagtcttcaaaagactttgatataactttctgtcatgatttaatagactacttcaaaaactgtattgcaattcatcccgagtggaaaaacttcggttttgattttagcgacaccagtacttatgaagacatttccgggttttatagagaggtagagttacaaggttacaagattgattggacatacattagcgaaaaagacattgatttattacaggaaaaaggtcaattatatttattccagatatataacaaagatttttcgaaaaaatcaaccgggaatgacaaccttcacaccatgtacttaaaaaatcttttctcagaagaaaatcttaaggatatagttttaaaacttaacggcgaagcggaaatattcttcaggaagagcagcataaagaacccaataattcataaaaaaggctcgattttagttaacagaacctacgaagcagaagaaaaagaccagtttggcaacattcaaattgtgagaaaaaatattccggaaaacatttatcaggagttatacaaatacttcaacgataaaagcgacaaagagttatctgatgaagcagccaaattaaagaatgtagtgggacaccacgaggcagcgacgaatatagttaaggactatagatacacgtatgataaatacttccttcatatgcctattacgataaatttcaaagccaataaaacgggttttattaatgataggatattacagtatatagctaaagaaaaagacttacatgtgataggcattgatagaggcgagagaaacttaatatacgtgtccgtgattgatacttgtggtaatatagttgaacagaaaagctttaacattgtaaacggctacgactatcagataaaattaaaacaacaggagggcgctagacagattgcgagaaaagaatggaaagaaattggtaaaattaaagagataaaagagggctacttaagcttagtaatacacgagatatctaaaatggtaataaaatacaatgcaattatagcgatggaggatttgtcttatggttttaaaaaagggagatttaaggttgaaagacaagtttaccagaaatttgaaaccatgttaataaataaattaaactatttagtatttaaagatatttcgattaccgagaatggcggtttattaaaaggttatcagttaacatacattcctgataaacttaaaaacgtgggtcatcagtgcggctgcattttttatgtgcctgctgcatacacgagcaaaattgatccgaccaccggctttgtgaatatatttaaatttaaagacttaacagtggacgcaaaaagagaattcattaaaaaatttgactcaattagatatgacagtgaaaaaaatttattctgctttacatttgactacaataactttattacgcaaaacacggttatgagcaaatcatcgtggagtgtgtatacatacggcgtgagaataaaaagaagatttgtgaacggcagattctcaaacgaaagtgataccattgacataaccaaagatatggagaaaacgttggaaatgacggacattaactggagagatggccacgatcttagacaagacattatagattatgaaattgttcagcacatattcgaaattttcagattaacagtgcaaatgagaaactccttgtctgaattagaggacagagattacgatagattaatttcacctgtattaaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgagagataaattaaaaataagcaataaagattggttcgactttatacagaataagagatatttataagtcgacaaagtattgttaaaaataactctgtagaattataaattagttctacagagttattttttgacccgggtatattgataaaaataataatagtgggtataattaagttgttaggaggttagttagaatgatgtcaagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaggtccactgccgggcctcttgcgggatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtccctaggcaggcctccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggc